(19) United States
(12) Patent Application Publication
(10) Pub. No.: US 2026/0248957 A1
(43) Pub. Date: Aug. 27, 2026

(54) ARRDC1-MEDIATED MICROVESICLE-BASED DELIVERY OF THERAPEUTIC AGENTS TO CELLS OF THE PERIPHERAL NERVOUS SYSTEM

(71) Applicant: Turn Biotechnologies, Inc., San Francisco, CA (US)

(72) Inventors: Joseph Nabhan, Cambridge, MA (US); Nedyalka Valkov, Cambridge, MA (US)

(21) Appl. No.: 18/874,840

(22) PCT Filed: Jun. 15, 2023
(86) PCT No.: PCT/US2023/068533

§ 371 Date: Dec. 13, 2024

Related U.S. Application Data
(60) Provisional application No. 63/352,544, filed on Jun. 15, 2022.

(51) Int. Cl. A61K 48/00; A61K 9/1271; A61K 47/64

(52) U.S. Cl. CPC A61K 48/0033; A61K 9/1271; A61K 47/64

(57) ABSTRACT

The present invention provides methods, systems, compositions, and strategies for the use of ARMM-mediated delivery of molecules (e.g, biological molecules, small molecules, proteins, and nucleic acids), to cells of the nervous system. In particular, the present invention generally relates to compositions and methods of producing, testing, and administering ARRDC1-mediated microvesicles (“ARMMs”) to peripheral nervous system cells in mammalian subjects.

### CLAIM OF PRIORITY

This application claims the benefit of U.S. Provisional Application Ser. No. 63/352,544 filed on Jun. 15, 2022. The entire contents of the foregoing are incorporated herein by reference.

### FIELD OF THE INVENTION

The present invention provides methods, systems, compositions, and strategies for the use of ARMM-mediated delivery of molecules (e.g., biological molecules, small molecules, proteins, and nucleic acids (e.g., DNA, RNA), DNA plasmids, siRNA, shRNA, mRNA, and the like), to cells of the nervous system (e.g., peripheral nervous system).

In particular, the present invention generally relates to compositions and methods of producing, testing, and administering ARRDC1-mediated microvesicles (“ARMMs”) to peripheral nervous system cells in mammalian subjects. More particularly, the present invention provides compositions and methods of producing, testing, and administering ARMMs particles comprising one or more therapeutic agents (e.g., biological molecules, small molecules, proteins, and nucleic acids (e.g., DNA, RNA), DNA plasmids, siRNA, shRNA, mRNA, and the like). Also provided are methods of administering therapeutic agents, including, but not limited to, treating, or contacting cells, tissues, and systems in one or more treatment environments (e.g., in vitro, in vivo, or ex vivo) with the invention compositions. In particular, the present invention provides methods of administering therapeutic agents via ARMMs to Schwann cells in mammalian subjects, including, but not limited to, humans. Additionally, the present invention relates to methods of creating, using, and harvesting the inventive compositions from producer cells and producer cell cultures.

### BACKGROUND OF THE INVENTION

Glia cells support neurons in the peripheral nervous system, including satellite cells, olfactory ensheathing cells, enteric glia cells, glia cells that reside at sensory nerve endings, such as the Pacinian corpuscle and the like.

Schwann cells, or neurolemmocytes, are the principal glia cells of the peripheral nervous system (“PNS”). Schwann cells develop from the neural crest (“NC”) via Schwann cell precursor (“SCP”) intermediates. In myelinated axons, Schwann cells form the myelin sheath. The sheath is not continuous, and individual Schwann cells wrap around about 100 μm of an axon. The gaps between adjacent Schwann cells are called nodes of Ranvier. The vertebrate nervous system is insulated with the myelin sheath to maintain the membrane capacitance of the axon. The action potential jumps from node to node of the nodes of Ranvier. Through this process, conduction velocity increases up to 10 times without increasing axonal diameter, and energy may be saved. Schwann cells may be the analogs of the oligodendrocytes, which play the same role in the central nervous system. However, unlike oligodendrocytes, Schwann cells form the myelin sheath in only one axon.

Schwann cells play crucial roles in functional regulation, maintenance, and repair of the nervous system. Consequently, healthy (non-pathogenic) Schwann cells are essential for proper nervous system function. Schwann cell defects are involved in a broad range of human disorders such as Schwannomatosis, Guillain Barre Syndrome (“GBS”), and other inherited peripheral neuropathies. Of these diseases and conditions, the inherited peripheral neuropathies represent a group of disorders that include hereditary motor neuropathy (“HMN”), hereditary sensory neuropathy (“HSN”), hereditary sensory and autonomic neuropathy (“HSAN”), and hereditary motor and sensory neuropathy (“HMSN”). HMSN, now known as Charcot-Marie-Tooth Disease (“CMT”), is the most observed of the inherited peripheral neuropathies.

CMT affects an estimated 126,000 individuals in the U.S. and 2.6 million people worldwide. Nearly all cases of CMT are inherited. It is possible to have two or more types of CMT (i.e., CMT1, CMT1A, CMT1B, CMT1C . . . , CMT2A, B, C . . . , CMT3, CMTX1, 2, 3 . . . , and CMT4, and the like), which results when the person has mutations in two or more genes, each of which causes a particular form of the disease. CMT is a heterogeneous genetic disease. Thus, mutations in different genes can produce heterogeneous clinical, electrophysiological, genetic, and pathological features in presentation.

CMT is a disorder that causes damage to the peripheral nerves. More particularly, CMT results from underlying abnormalities in the Schwann cells forming the insulating myelin sheathings of axons in the peripheral nervous system. CMT1A, specifically, is due to altered peripheral nerve myelin, resulting in demyelination and consequent aberrant saltatory conduction. The disease affects peripheral nerves that control the muscles in the patient's limbs and extremities, often leading to insidious progressive weakness in the affected muscles. The weakness associated with progressive CMT1A typically becomes noticeable in adolescence or early adulthood, but the onset of the disease can occur at any age. The disease may be severe when the onset is very early. The rate of progression varies among various CMTs.

Because longer nerves are affected first, symptoms usually begin in the lower extremities, the feet, and lower legs and later involve the upper extremities, the fingers, hands, and arms. (See, Fridman, V., et al., “Inherited Neuropathies Consortium. CMT subtypes and disease burden inpatients enrolled in the Inherited Neuropathies Consortium natural history study: a cross-sectional analysis” J. Neurol. Neurosurg. Psychiatry, 86(8):873-878 (2015); and Hoebeke, C., et. al., “Retrospective study of 75 children with peripheral inherited neuropathy: Genotype-phenotype correlations,” Arch. Pediatr., 25(8):452-458 (2018)). Most individuals with CMT1A have some amount of physical disability, although in some cases, people may never know they have the disease. CMT1A can also affect cranial nerves, other sites of the neuraxis, as well as other organ systems.

The art has long sought effective treatments and therapeutic agents for treating Schwann cell diseases such as CMT1A. For example, in treating CMT1A, various agents targeting PMP22 overexpression, including ascorbic acid, onapristone, geldanamycin, and rapamycin, have shown moderate success in animal models in improving muscle mass and slowing the progression of muscular weakness. However, these agents have failed to advance inhuman clinical trials for a number of reasons. (See, Mathis, S., et al., “Therapeutic options in Charcot-Marie-Tooth diseases,” Expert. Rev. Neurother., 15(4):355-366 (2015)).

In another study, anti-progesterone therapy significantly increased muscle strength and prevented axon loss in PMP22 transgenic rats, although myelin sheath thickness was not affected. Unfortunately, available progesterone antagonists are too toxic to administer to patients.

Neurotrophin-3 (NT3), a neurotrophic factor known to promote axonal growth, was tested with favorable results in two animal models, and in a pilot clinical trial involving eight CMT1A patients. Nevertheless, the development of this therapy has likewise ceased.

Currently, there are no therapeutic agents for treating diseases caused by dysfunction of Schwann cells, such as CMTs and, more particularly, CMT1A. There are only methods for partially reducing the aggravation of symptoms, such as physical therapy, orthopedic aids, and orthopedic surgery. Thus, while CMT1A is not typically life-threatening, the disease causes significant nerve pain, disabling loss of independence, safe ambulation, and other morbidities in many affected patients. A critical unmet need remains for developing efficacious therapies and treatments for ameliorating patients suffering from Schwann cell diseases such as CMT1A.

### SUMMARY OF THE INVENTION

This invention relates to the discovery that molecules, such as proteins and nucleic acids, including ribonucleic acids (RNAs), as well as small molecules, can be loaded into microvesicles, specifically ARRDC1-mediated microvesicles (ARMMs), for delivery to the nervous system, specifically cells of the PNS, and more specifically, to Schwann cells. In certain embodiments, the ARMMs can incorporate viral envelope proteins to allow for the delivery of molecules to the nervous system. For example, vesicular stomatitis virus G protein (VSV-G) or rabies virus glycoprotein (RVG) can be co-expressed in, and appear on, the surface of ARMMS to target cells of the PNS (e.g., Schwann cells). These proteins normally function to aid viral attachment and entry of viruses into cells. For example, VSV-G mediates viral attachment to LDL receptors (LDLR) or LDLR family members, and RVG is known to use the nicotinic acetylcholine receptor and the low affinity nerve growth factor receptor for viral entry. It has been found that these proteins can also aid ARMMs to attach to cells, including cells of the nervous system.

In addition, the ARMM delivery systems, described herein, address many limitations of current delivery systems that prevent the safe and efficient delivery of proteins and nucleic acids (e.g., RNAs (including both RNA coding for proteins and non-coding RNA) to CNS, and importantly, PNS cells. As ARMMs are derived from an endogenous budding pathway, they are unlikely to elicit a strong immune response, unlike viral delivery systems, which are known to trigger inflammatory responses. (See, Sen et al., “Cellular unfolded protein response against viruses used in gene therapy,” Front Microbiology, 5:250, 1-16 (2014)). Additionally, ARMMs allow for the specific packaging of many types and classes of potentially therapeutic molecules (e.g., biological molecules, such as a protein or nucleic acid (e.g., DNA plasmid, mRNA, miRNA, or shRNA), or small molecules). While the present invention is not intended to be limited to any particular mechanism(s), it is contemplated that ARMMs can be delivered by fusion with or uptake by, specific recipient cells and tissues by incorporating antibodies or other types of targeting or tropism determinant molecules into or onto the ARMMs so as to recognize tissue-specific markers.

ARMMs are microvesicles that are distinct from exosomes which, like budding viruses, are produced by direct plasma membrane budding (“DPMB”). DPMB is driven by a specific interaction of TSG101 with a tetrapeptide PSAP (SEQ ID NO: 1) motif of the arrestin-domain-containing protein ARRDC1 accessory protein, which is localized to the plasma membrane through its arrestin domain. ARMMs have been described in detail, for example, in PCT application number PCT/US2013/024839, filed Feb. 6, 2013 (published as WO 2013/119602 A1 on Aug. 15, 2013) by Lu, Q., et al., and entitled “Arrdc1-Mediated Microvesicles (ARMMs) and Uses Thereof,” as well as in U.S. Pat. Nos. 9,737,480; 9,816,080; 10,260,055; and PCT Publication WO2018/067546; the entire contents of which are hereby incorporated by reference in their entirety. The ARRDC1/TSG101 interaction results in relocation of TSG101 from endosomes to the plasma membrane and mediates the release of microvesicles that contain TSG101, ARRDC1, and other cellular components as well as the molecule of interest.

Molecules of interest, whether naturally, or non-naturally, occurring including, but not limited to, proteins, nucleic acids, and small molecules, can associate with one or more ARMM proteins (e.g., ARRDC1), or can be modified to associate with TSG101 or ARRDC1 or other motif(s) therein. This association facilitates their incorporation into ARMMs, which in turn can be used to deliver the desired payload (molecule of interest) into a targeted cell. By way of example, but not limitation, a payload RNA can be fused to a trans-activation response (TAR) element, thereby allowing it to associate with an ARRDC1 protein that is fused to an RNA binding protein, such as a Tat protein (e.g., bovine TAT protein). Alternatively, a payload protein can be fused to one or more WW domains, which associate with the PPXY (SEQ ID NO: 2) motif of ARRDC1. This association of the molecule of interest to an ARMM protein (e.g., ARRDC1), facilitates the loading of the molecule into the ARRDC1-containing ARMM. Alternatively, the molecule can be fused to an ARMM protein (e.g., TSG101 or ARRDC1) to load the payload into the ARMM. The molecule can be fused to the ARMM protein (e.g., TSG101 or ARRDC1) via a linker that may be cleaved upon delivery to a target cell.

Synthetic or natural small molecules and, more generally, therapeutic agents, can be modified to associate (e.g., covalently or non-covalently bind) with an ARMM protein (e.g., TSG101 or ARRDC1). This association can facilitate their incorporation into ARMMs, which in turn can be used to deliver the molecule to a target cell. The incorporation of a cleavable linker may be used to allow such a molecule to be released upon delivery into a target cell. As a non-limiting example, a small molecule can be linked to biotin, thereby allowing it to associate with an ARRDC1 protein which is fused to a streptavidin. As another non-limiting example, a small molecule can be linked to a synthetic high-affinity ligand that specifically binds to a mutant form of FKBP12 such as FKBP12(F36V) (See, Yang, W., et al., “Investigating protein-ligand interactions with a mutant FKBP possessing a designed specificity pocket,” J. Med. Chem., 23; 43(6):1135-1142 (2000)), which will associate with an ARRDC1 protein which is fused to FKBP12(F36V). In preferred embodiments, the association of the small molecule to an ARMM protein (e.g., TSG101 or ARRDC1), facilitates the loading of the small molecule into the ARRDC1-containing ARMM.

As an example, the delivery platform of ARMMs will enable multiple cis-acting structural elements of mRNAs to perform in the context of intracellular and secreted therapeutics for nervous system cells, where these structural elements include, but are not limited to: (i) 5′ cap structure; (ii) 5′ untranslated region (UTR); (iii) the codon optimized coding sequence; (iv) 3′ UTR; (v) a 3′ poly-A tail consisting of a stretch of repeated adenine nucleotides; and (vi) inclusion of cis-acting zipcode elements within RNA transcripts that are recognized by specific RNA binding proteins to cause specific cellular localization (e.g., to synapse of neurons). (See, e.g., Chin A., Lécuyer E., “RNA localization: Making its way to the center stage,” Biochim. Biophys. Acta. Gen Subj., 1861(11 Pt B):2956-2970 (2017)).

As another example, the delivery platform for ARMMs will enable multiple classes of protein and mRNA-based therapeutics to be targeted to nervous system cells, the therapeutics including, but not limited to: transmembrane proteins; cytoplasmic proteins; nuclear proteins; mitochondrial proteins; endoplasmic reticulum proteins; Golgi proteins; peroxisome proteins; lysosome proteins; and secreted proteins.

Also contemplated herein in the context of therapeutics for nervous system disorders are the targeted expression of single-chain variable fragment (scFv) antibodies composed of a fusion protein of the variable regions of the heavy (VH) and light chains (VL) of immunoglobulin connected with a short linker peptide. These scFv antibodies can bind selectively to a specific antigen or they can be engineered to be multifunctional by appending to the fusion other protein- or nucleic acid-biding domains, such as for example, the case of bispecific scFvs. Alternatively, mRNA encoding both VH and VL chains may be used. Additionally, a single-domain antibody (sdAb), consisting of a single monomeric variable domain, can be delivered to the nervous system cells as mRNAs. In additional embodiments, various other truncated antibodies and functional fragments thereof find use.

Also contemplated in the context of therapeutics for nervous system disorders is the targeted expression of antigenic peptides, or neoantigens, which can occur using ARMM-mediated delivery of a mRNA. The delivered mRNA is translated by the ribosome to produce a neoantigen protein chain which can be processed by the proteasome to produce a neoantigen. This neoantigen can associate with other membrane-bound proteins to display itself, thereby allowing it to be recognized by T-cell receptors on T-cells or other cells of the immune system.

In some aspects of this invention, arrestin domain-containing protein 1 (ARRDC1)-mediated microvesicles (ARMMs) containing a lipid bilayer and an ARRDC1 protein, a molecule (e.g., therapeutic agent(s)), and optionally a viral envelope protein are provided. In some aspects, the viral envelope protein is vesicular stomatitis virus G (VSV-G) or rabies virus glycoprotein (RVG).

In some aspects of the invention, microvesicle-producing cells containing a recombinant expression construct encoding an ARRDC1 protein or a variant thereof under the control of a heterologous promoter, and optionally, a viral envelope protein are provided. In other aspects, the viral envelope protein is vesicular stomatitis virus G (VSV-G) or rabies virus glycoprotein (RVG).

In some aspects of the invention, methods of delivering a molecule (e.g., one or more therapeutic agents) to a target cell by contacting the target cell with a microvesicle as described herein are provided. In other aspects, the cells are of the nervous system (NS), including the central nervous system (CNS) and the peripheral nervous system (PNS). In yet other aspects, the target cell is a neuron, astrocyte, an oligodendrocyte, or a microglial cell. In still other embodiments, the target cells are Schwann cells.

In some aspects of the invention, methods of treating a disorder in a patient by administering to the patient a microvesicle or a microvesicle-producing cell as described herein are provided. In yet other aspects, the disorder impacts the function of neurons, the function of astrocyte cells, the function of oligodendrocytes, the function of microglial cells, or the function of Schwann cells. In other aspects of the invention, the disorder is either a gain-of-function disorder, a loss-of-function disorder, or a repeat expansion.

In other aspects of the invention, the disorder is a disorder of the CNS system. In other preferred aspects of the invention, the disorder is a disorder of the PNS system. In still other more preferred aspects of the invention, the disorder is a disorder of the Schwann cells of the PNS system.

Other advantages, features, and uses of the invention will be apparent from the detailed description of certain exemplary, non-limiting embodiments, the drawings, the non-limiting working examples, and the claims.

### BRIEF DESCRIPTION OF THE DRAWINGS

FIG. 1 shows a non-limiting schematic of an ARMM-based development workflow for nervous system disorders involving the use of human induced pluripotent stem cells (iPSC) models for biological and therapeutic discovery and development. Such workflow can be adaptable to serve as a platform for the discovery and development of ARMM-based technologies and applications. In this schematic, arm skin biopsies are used to obtain iPSC-derived, post-mitotic (no longer dividing) neurons for use in screening and identifying ARMM-based therapeutics.

FIG. 2 is a non-limiting schematic for the screening of ARMM-mediated payload delivery using high-content, single-cell level imaging assays. In this schematic, the imaging is accomplished by automated confocal microscopy.

FIG. 3 is a non-limiting schematic of a workflow for designing ARMM-based technologies for development showing representative types of payloads and non-limiting CNS target cell types. Sequences shown: PSAP (SEQ ID NO: 1), PPXY (SEQ ID NO: 2), and

(SEQ ID NO: 57)
GGUCUCUCUGGUUAGACCAGAUCUGAGCCUGGGAGCUCUCUGGCUAACU
AGGGAACC

FIG. 4 shows the ARMMs-mediated delivery and expression of GFP mRNA to human iPSC-derived neural progenitor cells.

FIGS. 5A-5B show ARMMs-mediated delivery to and expression of GFP mRNA in (FIG. 5A) human iPSC-derived neural progenitor cells and (FIG. 513) human neurons derived from 3D iPSC cerebral organoids.

FIG. 6 shows successful ARMM-mediated delivery of an mRNA payload and translation of GFP protein in human neurons using high-content imaging. GFP protein expression, MAP2 staining, and nuclei staining represented in the imaging. As shown, ARMMs enable delivery of a payload to multiple subcellular regions of neurons, including axons, dendrites, and cell bodies.

FIG. 7 shows successful ARMM-mediated delivery of an mRNA payload and translation of GFP protein in human neurons. Sequences shown: PSAP (SEQ ID NO: 1), PPXY (SEQ ID NO: 2), and

(SEQ ID NO: 57)
GGUCUCUCUGGUUAGACCAGAUCUGAGCCUGGGAGCUCUCUGGCUAACU
AGGGAACC

FIG. 8 shows non-limiting, exemplary schematics for the use of ARMMs to target representative gain-of-function and loss-of-function mechanisms in neurogenetic disorders. These schematics show non-limiting examples of how ARMMs can be used to tailor personalized medicine to cells of the nervous system of a patient (neuropathology figures adapted from van der Zee J, Van Broeckhoven C. Dementia in 2013: frontotemporal lobar degeneration-building on breakthroughs. Nat Rev Neurol. 2014 February; 10(2):70-2). Sequences shown: PSAP (SEQ ID NO: 1), PPXY (SEQ ID NO: 2), and

(SEQ ID NO: 57)
GGUCUCUCUGGUUAGACCAGAUCUGAGCCUGGGAGCUCUCUGGCUAACU
AGGGAACC

FIG. 9 shows a non-limiting example of the therapeutic use of CRISPR/dCas9 activation for enhanced expression of the human GRN gene encoding progranulin relevant for the potential treatment of frontotemporal dementia caused by loss-of-function mutations in GRN or reduced progranulin expression, which is compatible with ARMM-delivery technology.

FIG. 10 shows a non-limiting example of a method for ARMM optimization for cells of the nervous system for FMRP delivery to rescue fragile X syndrome patient neurons.

FIG. 11 shows a non-limiting exemplary method of ARMM optimization for cells of the CNS for FMRP delivery to rescue fragile X syndrome patient neurons.

FIGS. 12A-12B show a non-limiting schematic of a fusion construct in which the RVG peptide along with a HA tag was inserted into the second extracellular loop of TSPAN6 (FIG. 12A), and a non-limiting, exemplary Western blot showing that TSPAN6-RVG-HA was robustly detected in ARMMs secreted from HEK293T cells (FIG. 12B).

FIGS. 13A-13B show non-limiting examples of methods for VSV-G insertion into ARMMs (FIG. 13A), and a non-limiting, exemplary Western blot showing that VSV-G was robustly detected in ARMMs secreted from HEK293T cells (FIG. 13B).

FIG. 14 shows a non-limiting example of ARRDC1-mediated delivery of payloads to cultured human iPSC-derived 3D cerebral organoids. Cerebral organoids were exposed to ARRDC1-GFP-VSVG ARMMs for either a 24 hr (top row) or 48 hr (bottom row) period. Inset shows the high percentage of green fluorescence protein (GFP) positive cells after dissociation and recovery 24 hours later.

FIGS. 15A-15C show various schematics of shRNA packaging strategies. FIG. 15A shows an exemplary construct comprising a Tat peptide (BIVtat65-81) fused via a linker to the C-terminus of ARRDC1. FIG. 15B shows an exemplary construct containing a Tat peptide (BIVtat65-81) fused via a linker to the C-terminus of ARRDC1 optionally further comprising a degron sequence. FIG. 15C shows an exemplary construct containing TAR was fused directly to the 5′ end of a cargo shRNA.

FIG. 16 shows successful packaging of TAR-shRNA targeting Pmp22 in ARMMs. ARRDC1-TAT was co-transfected with TAR-shRNA or control shRNA construct into Expi293F cells. ARMMs were pelleted via ultracentrifugation. Digital PCR was done on ARMMs to determine the shRNA copy numbers.

FIG. 17 shows successful delivery of TAR-shRNA into recipient Schwann-like cells (sNF02.2). Cells were incubated with ARMMs (1×10⁶ particles/cell) containing TAR-shRNA for 48 hrs, washed with PBS, and subjected to Pmp22 mRNA analysis by qRT-PCR.

FIG. 18 shows the dose-dependent effects of TAR-shRNA delivery into Schwann-like sNF02.2 recipient cells. sNF02.2 cells were incubated with increasing doses of ARMMs (1×10⁴, 1×10⁵, 1×10⁶ and 1×10⁷ particles/cell) containing TAR-shRNA for 48 hours, washed with PBS, and subjected to Pmp22 mRNA analysis by qRT-PCR.

FIG. 19 shows an exemplary TAR-shRNA construct

FIG. 20A shows the structure of the human PMP22 gene, wherein solid boxes indicate the relative positions of exons 1a, 1b, 2, 3, 4, and 5. The sequence of Promoter 1 (SEQ. ID. NO: 63) wherein the TATA box is highlighted. The protospacer for gRNA5 is underlined.

FIG. 20B shows an exemplary Western blot of payloading of ABE8 into ARMMs in one embodiment. WCL: whole cell lysate of producer cells.

FIG. 20C shows exemplary editing efficiency achieved by ARMMs in human primary Schwann cells at adenine sites A2, A3, A5, A6, and A7 within the editing window by ARMMs payloaded with ABE8-PMP22 TATA gRNA5.

FIG. 21 shows an exemplary schematic of human PMP22 gene structure and transcripts, illustrating that the expression of transcripts 1 and 5 is driven by promoter 1 whereas that of transcript 2 and 4 by promoter 2.

FIG. 22A shows exemplary editing status of human primary Schwann cells (hPSCs) in differentiation. High percentage editing was confirmed in the PMP22 TATA box in the ARMMs-treated cells (ABE8-PMP22gRNA5), whereas editing was seen in the control cells (Ctrl).

FIG. 22B shows exemplary editing expression of various transcripts measured in differentiating hPSCs. Reduced expression is detected in the ABE8-PMP22gRNA5hPSCs for transcripts 1 and 5 driven by promoter 1 but not for transcripts 2 and 4 driven by promoter 2.

### DEFINITIONS

The term “ARMM,” as used herein, refers to a microvesicle comprising an ARRDC1 protein or variant thereof, and/or TSG101 protein, or variant thereof. In some embodiments, the ARMM is shed from a cell (i.e., producer cell), and comprises a payload, for example, comprising a nucleic acid, protein, or small molecule, present in the cytoplasm or associated with the membrane of the cell. In some embodiments, the ARMM is shed from a transgenic cell comprising a recombinant expression construct that includes a transgene, and the ARMM comprises a gene product, for example, an RNA transcript and/or a protein (e.g., an ARRDC1-Tat fusion protein and a TAR-payload RNA) encoded by the expression construct. It is contemplated that the high binding affinity between the TAR and the Tat peptide will allow the recruitment of the Tar-fused RNA (e.g., shRNA) into ARMMs for substantial benefit. In some embodiments, the ARMM is produced synthetically, for example, by contacting a lipid bilayer with an ARRDC1 protein, or variant thereof, in a cell-free system in the presence of TSG101, or a variant thereof. In other embodiments, the ARMM is synthetically produced by contacting a lipid bilayer with HECT domain ligase, and VPS4a. In some embodiments, an ARMM lacks a late endosomal marker. Some of the ARMMs provided herein do not include, or are negative for, one or more exosomal biomarkers. Exosomal biomarkers are known in the art and include, but are not limited to, CD63, Lamp-1, Lamp-2, CD9, HSPA8, GAPDH, CD81, SDCBP, PDCD6IP, ENO1, ANXA2, ACTB, YWHAZ, HSP90AA1, ANXA5, EEF1A1, YWHAE, PPIA, MSN, CFL1, ALDOA, PGK1, EEF2, ANXA1, PKM2, HLA-DRA, and YWHAB. Certain ARMMs provided herein may include an exosomal biomarker. Accordingly, some ARMMs may be negative for one or more other exosomal biomarkers, but positive for one or more different exosomal biomarkers. For example, such an ARMM may be negative for CD63 and Lamp-1, but may include PGK1 or GAPDH, or may be negative for CD63, Lamp-1, CD9, and CD81, but may be positive for HLA-DRA. In some embodiments, ARMMs include an exosomal biomarker, but at a lower level than the level found in exosomes. For example, some ARMMs include one or more exosomal biomarkers at a level of less than about 1%, less than about 5%, less than about 10%, less than about 20%, less than about 30%, less than about 40%, or less than about 50% of the level of that biomarker found in exosomes. To give a non-limiting example, in some embodiments, an ARMM may be negative for CD63 and Lamp-1, include CD9 at a level of less than about 5% of the level of CD9 typically found in exosomes, and be positive for ACTB. Exosomal biomarkers in addition to those listed above are known to those of skill in the art, and the invention is not limited in this regard.

The term “binding RNA,” as used herein, refers to a ribonucleic acid (RNA) that binds to an RNA binding protein, for example, any of the RNA binding proteins known in the art and/or described herein. In some embodiments, a binding RNA is an RNA that specifically binds to an RNA binding protein. A binding RNA that “specifically binds” to an RNA binding protein, binds to the RNA binding protein with greater affinity, avidity, more readily, and/or with greater duration than it binds to another protein, such as a protein that does not bind the RNA or a protein that weakly binds to the binding RNA. In some embodiments, the binding RNA is a naturally-occurring RNA, or non-naturally-occurring variant thereof, that binds to a specific RNA binding protein. For example, the binding RNA may be a TAR element, a Rev response element, an MS2 RNA, or any variant thereof that specifically binds an RNA binding protein. In some embodiments, the binding RNA may be a trans-activating response element (TAR element), or variant thereof, which is an RNA stem-loop structure that is found at the 5′-ends of nascent HIV-1 transcripts and specifically binds to the trans-activator of transcription (Tat) protein. In some embodiments, the binding RNA is a Rev response element (RRE), or variant thereof, that specifically binds to the accessory protein Rev (e.g., Rev from HIV-1). In some embodiments, the binding RNA is an MS2 RNA that specifically binds to a MS2 phage coat protein. The binding RNAs of the present disclosure may be designed to specifically bind a protein (e.g., an RNA binding protein fused to ARRDC1) to facilitate loading of the binding RNA (e.g., a binding RNA fused to a payload RNA) into an ARMM.

The term “aptamer,” as used herein, refers to nucleic acids (e.g., RNA, DNA) that bind to a specific target molecule, e.g., an RNA binding protein. In some embodiments, nucleic acid (e.g., DNA or RNA) aptamers are engineered through repeated rounds of in vitro selection or alternatively, SELEX (systematic evolution of ligands by exponential enrichment) methodology, to bind to various molecular targets, for example, proteins, small molecules, macromolecules, metabolites, carbohydrates, metals, nucleic acids, cells, tissues, and organisms. Methods for engineering aptamers to bind to various molecular targets, such as proteins, are known in the art and include those described in U.S. Pat. Nos. 6,376,19; and 9,061,043; Shui, B., et al., “RNA aptamers that functionally interact with green fluorescent protein and its derivatives,” Nucleic Acids Res., March; 40(5): e39 (2012); Trujillo, U. H., et al., “DNA and RNA aptamers: from tools for basic research towards therapeutic applications,” Comb. Chem. High Throughput Screen, 9(8):619-32 (2006); Srisawat, C., et al., “Streptavidin aptamers: Affinity tags for the study of RNAs and ribonucleoproteins,” RNA, 7:632-641 (2001); and Tuerk and Gold, “Systematic evolution of ligands by exponential enrichment: RNA ligands to bacteriophage T4 DNA polymerase,” Science, (1990); the entire contents of each of which are hereby incorporated by reference in their entirety.

The term “RNA binding protein,” as used herein, refers to a polypeptide molecule that binds to a binding RNA, for example, any of the binding RNAs known in the art and/or described herein. In some embodiments, an RNA binding protein is a protein that specifically binds to a binding RNA. An RNA binding protein that “specifically binds” to a binding RNA, binds to the binding RNA with greater affinity, avidity, more readily, and/or with greater duration than it binds to another RNA, such as a control RNA (e.g., an RNA having a random nucleic acid sequence) or an RNA that weakly binds to the RNA binding protein. In some embodiments, the RNA binding protein is a naturally-occurring protein, or a non-naturally occurring variant thereof, that binds to a specific RNA. For example, in some embodiments, the RNA binding protein may be a trans-activator of transcription (Tat) protein that specifically binds a trans-activating response element (TAR element). In some embodiments, the Tat protein is from a bovine. In some embodiments, the RNA binding protein is a regulator of virion expression (Rev) protein (e.g., Rev from HIV-1) or variant thereof, that specifically binds to a Rev response element (RRE). In some embodiments, the RNA binding protein is a coat protein of an MS2 bacteriophage that specifically binds to an MS2 RNA. The RNA binding proteins useful in the present disclosure (e.g., a binding protein fused to ARRDC1) may be designed to specifically bind a binding RNA (e.g., a binding RNA fused to a payload RNA) to facilitate loading of the binding RNA into an ARMM.

The term “payload,” “payload protein,” “payload nucleic acid,” “payload DNA,” “payload RNA,” or “payload small molecule,” as used herein, refers to a protein, nucleic acid, including DNA or RNA, or a small molecule, respectively, that may be incorporated into an ARMM, for example, into the liquid phase of the ARMM or into the lipid bilayer of an ARMM. Types of payload protein, payload nucleic acid, payload DNA, payload RNA, and payload small molecule are known in the art and include those described in U.S. Pat. Nos. 9,737,480; 9,816,080; 10,260,055; and PCT Publication WO2018/067546; the entire contents of each of which are hereby incorporated by reference in their entirety.

The payload can be delivered via its association with or inclusion in an ARMM to a subject, organ, tissue, or cell. In some embodiments, the payload is to be delivered to a targeted cell in vitro, in vivo, or ex vivo. In some embodiments, the payload to be delivered is a biologically active agent, i.e., it has activity in a cell, organ, tissue, and/or subject. For instance, a protein, nucleic acid (e.g., DNA or RNA), or small molecule that, when administered to a subject, has a biological effect on that subject or is considered to be biologically active. In some embodiments, a payload to be delivered is a therapeutic agent.

As used herein, the term “therapeutic agent” refers to any agent that, when administered to a subject has a beneficial effect. In some embodiments, the payload comprises a small molecule, a protein (or peptide), one or more nucleic acids, or an agent associated with a small molecule. In some embodiments, the payload to be delivered is a diagnostic agent. In some embodiments, the payload to be delivered is a prophylactic agent. In some embodiments, the payload to be delivered is useful as an imaging agent. In some of these embodiments, the diagnostic or imaging agent is, and in others, it is not, biologically active. In some embodiments, the therapeutic agent comprises an agent that reduces (knocks down) the expression of one or more genes in an organism (e.g., a subject). In other embodiments, the therapeutic agent comprises an agent that inactivates or removes (knocks out) one or more specific genes in an organism (e.g., a subject).

The terms “nervous system” or “NS,” as used herein, refers collectively to the CNS and the PNS. Cells of the nervous system include neurons, astrocytes, oligodendrocytes, and microglia with further interaction with endothelial cells in blood vessels and cells of the immune system including T-cells. While neurons, astrocytes, and oligodendrocytes are terminally differentiated cells, in certain niches of the CNS neural stem and neural progenitor cells exist that retain the capacity to replicate through both symmetric and symmetric cell division to produce additional stem cells, progenitor cells, and cells that will terminally differentiated into neurons and astrocytes. Microglia, the resident immune system cells of the nervous system, are also able to proliferate.

The term “central nervous system,” or “CNS,” as used herein, is the portion of the nervous system comprised of the cells and tissues of the brain and spinal cord. Two of the major types of cells that make up the nervous system are neurons and glial cells. In some embodiments, neurons are excitatory, and in some embodiments they are inhibitory. In some embodiments, the cells of the CNS include, but are not necessarily limited to, neuronal cells, glial cells, oligodendrocytes, astrocytes, microglia, cerebrospinal fluid (CSF), interstitial spaces, bone, cartilage, and the like.

The terms “peripheral nervous system,” or “PNS,” as may be used herein, refer to all cells and tissue of the nervous system outside of the cells and tissues of the brain and spinal cord (i.e., outside of the CNS). The PNS consists of the nerves and ganglia outside of the CNS comprised primarily of two types of cells in the peripheral nervous system. Sensory nervous cells carry information to the CNS and motor nervous cells carry information from the CNS. The PNS also includes, in context, Schwann cells, which myelinate the cells of the nervous system. Cells of the sensory nervous system send information to the CNS from internal organs or from external stimuli. Motor nervous system cells carry information from the CNS to organs, muscles, and glands.

As used herein, the term “Schwann cell” refers to a cell that expresses one or more Schwann cell marker(s), which include, but are not limited to, the Schwann cell markers disclosed herein. The Schwann cell can be a myelinating Schwann cell or a non-myelinating Schwann cell. In certain embodiments, Schwann cells are capable of maintaining and regenerating axons of the neurons in the peripheral nervous system (e.g., maintenance of healthy axons). In certain other embodiments, the Schwann cells are capable of forming myelin sheaths. In certain further embodiments, the Schwann cells are capable of forming Remak bundles. Schwann cell markers within the meaning of the invention, include but are not limited to: LRRTM4, CDH1, FABP7, BDNF, UNCB5, SOSTDC1, OLIG1, PLAT, KCNJ10, SHH, NTN1, GDNF, ERBB3, GAP43, SOX10, 5100, GFAP, POU3F1, PMP22, MBP, AQP4, MPZ, NGFR, NFATC4, MOG, IFNG, MAL, NTF3, TGFB1, CD9, CD81, CD44, CD98, CD49E, CD49D, TYRP1, ENTHD1, NTSE, HTR2B, NOV, IL8, SLC16A6, CDKN2A, PLP2, S100A6, AQP9, and CDH19. Additionally, U.S. Pat. Publication No.: 20190331666, incorporated herein by reference in its entirety, discloses several suitable Schwann cell markers that find use in various compositions and methods of the present invention.

The term “viral envelope proteins” refers to proteins that normally function to aid viral attachment and entry into cells. In some embodiments, viral envelope proteins can be incorporated into ARMMS to allow for the targeting of cells of the CNS. Non-limiting examples of viral envelope proteins include vesicular stomatitis virus G protein (VSV-G) or rabies virus glycoprotein (RVG). VSV-G mediates viral attachment to LDL receptors (LDLR) or LDLR family member, and RVG is known to use the nicotinic acetylcholine receptor and the low affinity nerve growth factor receptor for viral entry.

The term “linker,” as used herein, refers to a chemical moiety linking two molecules or moieties, e.g., an ARRDC1 protein and a Tat protein, a WW domain and a Tat protein, or an ARRDC1 protein and a Cas9 nuclease. Typically, the linker is positioned between, or flanked by, two groups, molecules, or other moieties and connected to each one via a covalent bond, thus connecting the two. In some embodiments, the linker comprises an amino acid or a plurality of amino acids (e.g., a peptide or protein). In some embodiments, the linker comprises a nucleotide (e.g., DNA or RNA) or a plurality of nucleotides (e.g., a nucleic acid). In some embodiments, the linker is an organic molecule, functional group, polymer, or other chemical moiety/moieties. In some embodiments, the linker is a cleavable linker, e.g., the linker comprises a bond that can be cleaved upon exposure to, for example, UV light or a hydrolytic enzyme, such as a protease or esterase. In some embodiments, the linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids). In other embodiments, the linker is a chemical bond (e.g., a covalent bond, amide bond, disulfide bond, ester bond, carbon-carbon bond, carbon-heteroatom bond, and the like).

As used herein, the term “animal” refers to any member of the animal kingdom. In some embodiments, the term “animal” refers to a human of either sex at any stage of development. In some embodiments, the term “animal” refers to a non-human animal at any stage of development. In certain embodiments, the non-human animal is a mammal (e.g., a rodent, a mouse, a rat, a rabbit, a monkey, a dog, a cat, a sheep, cattle, a primate, or a pig). Animals include, but are not limited to, mammals, birds, reptiles, amphibians, fish, and worms. In some embodiments, the animal is a transgenic animal, a genetically-engineered animal, or a clone. In some embodiments, the animal is a transgenic non-human animal, a genetically-engineered non-human animal, or a non-human clone.

As used herein, the term “approximately” or “about,” as applied to one or more values of interest, refers to a value that is similar to a stated reference value. In certain embodiments, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (for example, when such number would exceed 100% of a possible value).

As used herein, the term “associated with,” when used with respect to two or more entities, for example, with chemical moieties, molecules, and/or ARMMs, means that the entities are physically associated or connected with one another, either directly or via one or more additional moieties that serve as a linker, to form a structure that is sufficiently stable so that the entities remain physically associated under the conditions in which the structure is used, e.g., under physiological conditions. An ARMM is typically associated with an agent, for example, a nucleic acid, protein, or small molecule, by a mechanism that involves a covalent (e.g., via an amide bond) or non-covalent association (e.g., between ARRDC1 and a WW domain, or between a Tat protein and a TAR element). In certain embodiments, the agent to be delivered (e.g., a payload protein, payload nucleic acid, or payload small molecule) is covalently bound to a molecule that associates non-covalently with a part of the ARMM that is fused to an ARRCD1 protein, or variant thereof. In some embodiments, the association is via a linker, for example, a cleavable linker. In some embodiments, an entity (e.g., a payload protein, payload nucleic acid, or payload small molecule) is associated with an ARMM by inclusion in the ARMM, for example, by encapsulation of the molecule within the ARMM. For example, in some embodiments, a molecule (e.g., a payload protein, payload nucleic acid, or payload small molecule) present in the cytoplasm of an ARMM-producing cell is associated with an ARMM by encapsulation of the cytoplasm with the agent in the ARMM upon ARMM budding. Similarly, a membrane protein or other molecule associated with the cell membrane of an ARMM producing cell may be associated with an ARMM produced by the cell by inclusion into the ARMM's membrane upon budding.

As used herein, the phrase “biologically active” refers to a characteristic of any substance that has activity in a cell, organ, tissue, and/or subject. For instance, a substance that, when administered to an organism, has a biological effect on that organism, is considered to be biologically active. As one example, a payload RNA may be considered biologically active if it increases or decreases the expression of a gene product when administered to a subject or cell. As another example, a nuclease payload protein may be considered biologically active if it increases or decreases the expression of a gene product when administered to a subject.

As used herein, the term “conserved” refers to nucleotides or amino acid residues of a polynucleotide sequence or amino acid sequence, respectively, that are those that occur unaltered in the same position of two or more related sequences being compared. Nucleotides or amino acids that are relatively conserved are those that are conserved amongst more related sequences than nucleotides or amino acids appearing elsewhere in the sequences. In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95% identical, about 98% identical, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another.

The term “engineered,” as used herein, refers to a protein, nucleic acid, complex, substance, or entity that has been designed, produced, prepared, synthesized, and/or manufactured by a human. Accordingly, an engineered product is a product that does not occur in nature. In some embodiments, an engineered protein or nucleic acid is a protein or nucleic acid that has been designed to meet particular requirements or to have particular design features. For example, a payload RNA may be engineered to associate with the ARRDC1 by fusing one or more WW domains to a Tat protein and fusing the payload RNA to a TAR element to facilitate loading of the payload RNA into an ARMM. As another example, a payload RNA may be engineered to associate with the ARRDC1 by fusing a Tat protein to the ARRDC1 and by fusing the payload RNA to a TAR element to facilitate loading of the payload RNA into an ARMM. As another example, a payload protein may be engineered to associate with the ARRDC1 by fusing one or more WW domains to the payload protein to facilitate the loading of the payload protein into an ARMM.

As used herein, “expression” of a nucleic acid sequence refers to one or more of the following events: (1) production of an RNA transcript from a DNA sequence (e.g., by transcription); (2) processing of an RNA transcript (e.g., by splicing, editing, 5′ cap formation, and/or 3′ end processing); (3) translation of an RNA transcript into a polypeptide or protein; and (4) post-translational modification of a polypeptide or protein.

As used herein, a “fusion protein” includes a first protein moiety, e.g., an ARRCD1 protein or variant thereof, or a TSG101 protein or variant thereof, associated with a second protein moiety, for example, a protein to be delivered to a target cell through a peptide linkage. In certain embodiments, the fusion protein is encoded by a single fusion gene.

As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that the definition of a gene includes references to nucleic acids that do not encode proteins but rather encode functional RNA molecules, such as gRNAs, RNAi agents, ribozymes, tRNAs, etc. For the purpose of clarity, it should be noted that, as used in the present application, the term “gene” generally refers to a portion of a nucleic acid that encodes a protein; the term may optionally encompass regulatory sequences, as will be clear from context to those of ordinary skill in the art. This definition is not intended to exclude the application of the term “gene” to non-protein-coding expression units but rather to clarify that, in most cases, the term as used herein refers to a protein-coding nucleic acid.

As used herein, the term “gene product” or “expression product” generally refers to an RNA transcribed from the gene (pre- and/or post-processing) or a polypeptide (pre- and/or post-modification) encoded by an RNA transcribed from the gene.

As used herein, the term “green fluorescent protein” (GFP) refers to a protein originally isolated from the jellyfish Aequorea victoria that fluoresces green when exposed to blue light or a derivative of such a protein (e.g., an enhanced or wavelength-shifted version of the protein). The amino acid sequence of wild type GFP is as follows:

(SEQ ID NO: 3)
MSKGEELFTGVVPILVELDGDVNGHKESVSGEGEGDATYGKLTLKFICT
TGKLPVPWPTLVTTFSYGVQCFSRYPDHMKQHDFFKSAMPEGYVQERTI
FFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLP
DNHYLSTQSALSKDPNEKRDHMVLLEFVTAAGITHGMDELYK

Proteins that are at least 70%, at least 75%, at least 80%, at least 85%, at least 90%, at least 95%, at least 98%, or at least 99% homologous to SEQ ID NO: 3 are also considered to be green fluorescent proteins.

As used herein, the term “homology” refers to the overall relatedness between nucleic acids (e.g., DNA molecules and/or RNA molecules) or polypeptides. In some embodiments, nucleic acids or proteins are considered to be “homologous” to one another if their sequences are at least 50%, at least 55%, at least 60%, at least 65%, at least 70%, at least 75%, at least 80%, at least 85%, at least 90%, at least 95%, or at least 99% identical. In some embodiments, nucleic acids or proteins are considered “homologous” to one another if their sequences are at least 50%, at least 55%, at least 60%, at least 65%, at least 70%, at least 75%, at least 80%, at least 85%, at least 90%, at least 95%, or at least 99% identical. The term “homologous” necessarily refers to a comparison between at least two sequences (nucleotide sequences or amino acid sequences). In accordance with the invention, two nucleotide sequences are considered homologous if the polypeptides they encode are at least about 50% identical, at least about 60% identical, at least about 70% identical, at least about 80% identical, or at least about 90% identical for at least one stretch of at least about 20 amino acids. In some embodiments, homologous nucleotide sequences are characterized by the ability to encode a stretch of at least 4-5 uniquely specified amino acids. Both the identity and the approximate spacing of these amino acids relative to one another must be considered for sequences to be considered homologous. For nucleotide sequences less than 60 nucleotides in length, homology is determined by the ability to encode a stretch of at least 4-5 uniquely specified amino acids. In accordance with the invention, two protein sequences are considered homologous if the proteins are at least about 50% identical, at least about 60% identical, at least about 70% identical, at least about 80% identical, or at least about 90% identical for at least one stretch of at least about 20 amino acids.

As used herein, the term “identity” refers to the overall relatedness between nucleic acids or proteins (e.g., DNA molecules, RNA molecules, and/or polypeptides). Calculation of the percent identity of two nucleic acid sequences, for example, can be performed by aligning the two sequences for optimal comparison purposes (e.g., gaps can be introduced in one or both of a first and second nucleic acid sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smth, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Atschul, S. F., et al., J. Molec. Biol., 215, 403 (1990)).

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe).

As used herein, the term “ex vivo” refers to events outside of the living body and thusly is understood to refer to medical procedures in which an organ, cells, or tissue is taken from a living body for a treatment or procedure, and then returned to the same, or another, living body. In certain embodiments, ex vivo therapy comprises inducing one or more genetic modifications in a patient's cells outside of their body to produce therapeutic effects therein and the subsequent transfer (e.g., transplantation) of the cells back into the patient.

As used herein, the term “isolated” refers to a substance or entity that has been: (1) separated from at least some of the components with which it was associated when initially produced (whether in nature or in an experimental setting); and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated substances are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

As used herein, the term “nucleic acid,” in its broadest sense, refers to a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleotides. As used herein, the terms “oligonucleotide” and “polynucleotide” can be used interchangeably to refer to a polymer of nucleotides (e.g., a string of at least two nucleotides). In some embodiments, “nucleic acid” encompasses RNA as well as single and/or double-stranded DNA and/or complementary DNA (cDNA). Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified to facilitate or achieve delivery.

As used herein, the term “protein” refers to a string of at least two amino acids linked to one another by one or more peptide bonds. Proteins may include moieties other than amino acids (e.g., may be glycoproteins) and/or may be otherwise processed or modified. Those of ordinary skill in the art will appreciate that a “protein” can be a complete protein chain as produced by a cell (with or without a signal sequence) or can be a functional portion thereof. Those of ordinary skill will further appreciate that a protein can sometimes include more than one protein chain, for example linked by one or more disulfide bonds or associated by other means. Proteins may contain L-amino acids, D-amino acids, or both and may contain any of a variety of amino acid modifications or analogs known in the art. Useful modifications include, e.g., addition of a chemical entity such as a carbohydrate group, a phosphate group, a farnesyl group, an isofarnesyl group, a fatty acid group, an amide group, a terminal acetyl group, a linker for conjugation, functionalization, or other modification (e.g., alpha amidation), etc. In certain embodiments, the modifications of the protein lead to a more stable protein (e.g., greater half-life in vivo). These modifications may include cyclization of the protein, the incorporation of D-amino acids, etc. None of the modifications should substantially interfere with the desired biological activity of the protein. In certain embodiments, the modifications of the protein lead to a more biologically active protein. In some embodiments, proteins may comprise natural amino acids, non-natural amino acids, synthetic amino acids, amino acid analogs, and combinations thereof.

As used herein, the terms “subject,” or “patient” refer to any organism to which a composition in accordance with the invention may be administered, e.g., for experimental, diagnostic, prophylactic, and/or therapeutic purposes. Typical subjects include animals (e.g., mammals, such as mice, rats, rabbits, production and farm animals, pets, non-human primates, and humans). In some embodiments, the subject is a patient having or suspected of having a disease or disorder. In other embodiments, the subject is a healthy volunteer.

As used herein, the terms “disease,” or “disorder” refer to any condition, pathological condition, or disorder that damages or interferes with the normal function of a cell, tissue, or organ.

As used herein, the terms “treating,” or “treatment” refer to partially or completely preventing, altering, and/or reducing the incidence of one or more symptoms or features of a particular disease or condition. In one sense of the invention, treatments can be performed either for prophylaxis or during clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. Treatment may be administered to a subject who does not exhibit signs or symptoms of a disease, disorder, and/or condition and/or to a subject who exhibits only early signs or symptoms of a disease, or condition for the purpose of decreasing the risk of developing or progressing to more severe effects associated with the disease, disorder, or condition. A treatment may prevent the onset of the disorder or a symptom of the disorder in a subject. A treatment can prevent physical deterioration (e.g., weakening of muscles) caused by a disorder (e.g., CMT1A) by preventing its progression. In an additional sense of the invention, “treating,” in regard to cancer, may refer to inhibiting the survival, growth, and/or spread of the cancer. In yet another sense of the invention, “treating,” in regard to a benign tumor, may refer to inhibiting the survival or growth of the tumor.

As used herein, the term “therapeutically effective amount” means an amount of an agent to be delivered (e.g., nucleic acid, protein, drug, therapeutic agent, diagnostic agent, prophylactic agent, ARMM, or ARMM comprising a payload protein or payload RNA) that is sufficient, when administered to a subject suffering from or susceptible to a disease, disorder, and/or condition, to treat, improve symptoms of, diagnose, prevent, and/or delay the onset of the disease, disorder, and/or condition.

As used herein, a “vector” means any nucleic acid or nucleic acid-bearing particle, cell, or organism capable of being used to transfer a nucleic acid into a host cell. The term “vector” includes both viral and nonviral products and means for introducing the nucleic acid into a cell. A “vector” can be used in vitro, ex vivo, or in vivo. Vectors capable of directing the expression of operatively linked genes are referred to herein as “expression vectors.” Non-viral vectors include plasmids, cosmids, artificial chromosomes (e.g., bacterial artificial chromosomes or yeast artificial chromosomes), liposomes, electrically charged lipids (cytofectins), DNA-protein complexes, and biopolymers, for example. Viral vectors include, but are not limited to, retroviruses, lentiviruses, adeno-associated virus, pox viruses, baculovirus, reoviruses, vaccinia viruses, herpes simplex viruses, Epstein-Barr viruses, and adenovirus vectors, for example. Vectors can also comprise the entire genome sequence or recombinant genome sequence of a virus. A vector can also comprise a portion of the genome that comprises the functional sequences for the production of a virus capable of infecting, entering, or being introduced to a cell to deliver nucleic acid therein.

The term “WW domain” as used herein, refers to a protein domain having two basic residues at the C-terminus that mediates protein-protein interactions with short proline-rich or proline-containing motifs. It should be appreciated that the two basic residues (e.g., any two of: H, R, and K) of the WW domain are not required to be at the absolute C-terminal end of the WW protein domain. Rather, the two basic residues may be at a C-terminal portion of the WW protein domain (e.g., the C-terminal half of the WW protein domain). In some embodiments, the WW domain contains at least 1, 2, 3, 4, 5, 6, 7, 8, 9, or 10 tryptophan (W) residues. In some embodiments, the WW domain contains at least two W residues. In some embodiments, the at least two W residues are spaced apart by from 15-25 amino acids. In some embodiments, the at least two W residues are spaced apart by from 19-23 amino acids. In some embodiments, the at least two W residues are spaced apart by from 20-22 amino acids. The WW domain possessing the two basic C-terminal amino acid residues may have the ability to associate with short proline-rich or proline-containing motifs (e.g., a PPXY (SEQ ID NO: 2) motif). WW domains bind a variety of distinct peptide ligands including motifs with core proline-rich sequences, such as PPXY (SEQ ID NO: 2), which is found in ARRDC1. A WW domain may be a 30-40 amino acid protein interaction domain with two signature tryptophan residues spaced by 20-22 amino acids. The three-dimensional structure of WW domains shows that they generally fold into a three-stranded, antiparallel R sheet with two ligand-binding grooves.

WW domains are found in many eukaryotes and are present in approximately 50 human proteins (Bork, P. & Sudol, M., “The WW domain: a signaling site in dystrophin?,” Trends Biochem Sci., 19, 531-533 (1994)). WW domains may be present together with several other interaction domains, including membrane targeting domains, such as C2 in the NEDD4 family proteins, the phosphotyrosine-binding (PTB) domain in FE65 protein, FF domains in CA150 and FBPII, and pleckstrin homology (PH) domains in PLEKHA5. WW domains are also linked to a variety of catalytic domains, including HECT E3 protein-ubiquitin ligase domains in NEDD4 family proteins, rotomerase or peptidyl prolyisomerase domains in Pinl, and Rho GAP domains in ArhGAP9 and ArhGAP12.

In the instant disclosure, the WW domain may be a WW domain that naturally possesses two basic amino acids at the C-terminus. In some embodiments, a WW domain or WW domain variant may be from the human ubiquitin ligase WWP1, WWP2, Nedd4-1, Nedd4-2, Smurf1, Smurf2, ITCH, NEDL1, or NEDL2. Exemplary amino acid sequences of WW domain containing proteins (WW domains underlined) are listed below. It should be appreciated that any of the WW domains or WW domain variants of the exemplary proteins may be used in the invention, described herein, and are not meant to be limiting.

Human WWP1 amino acid sequence (uniprot.org/uniprot/Q9H0M0). The four underlined WW domains correspond to amino acids 349-382 (WW1), 381-414 (WW2), 456-489 (WW3), and 496-529 (WW4).

| (SEQ ID NO: 4) | |
|---|---|
| MATASPRSDT SNNHSGRLQL QVTVSSAKLK RKKNWFGTAI YTEVVVDGEI | 50 |
| TKTAKSSSSS NPKWDEQLTV NVTPQTTLEF QVWSHRTLKA DALLGKATID | 100 |
| LKQALLIHNR KLERVKEQLK LSLENKNGIA QTGELTVVLD GLVIEQENIT | 150 |
| NCSSSPTIEI QENGDALHEN GEPSARTTAR LAVEGTNGID NHVPTSTLVQ | 200 |
| NSCCSYVVNG DNTPSSPSQV AARPKNTPAP KPLASEPADD TVNGESSSFA | 250 |
| PTDNASVTGT PVVSEENALS PNCTSTTVED PPVQEILTSS ENNECIPSTS | 300 |
| AELESEARSI LEPDTSNSRS SSAFEAAKSR QPDGCMDPVR QQSGNANTET | 350 |
| LPSGWEQRKD PHGRTYYVDH NTRTTTWERP QPLPPGWERR VDDRRRVYYV | 400 |
| DHNTRTTTWQ RPTMESVRNF EQWQSQRNQL QGAMQQFNQR YLYSASMLAA | 450 |
| ENDPYGPLPP GWEKRVDSTD RVYFVNHNTK TTQWEDPRTQ GLQNEEPLPE | 500 |
| GWEIRYTREG VRYFVDHNTR TTTFKDPRNG KSSVTKGGPQ IAYERGFRWK | 550 |
| LAHFRYLCQS NALPSHVKIN VSRQTLFEDS FQQIMALKPY DLRRRLYVIF | 600 |
| RGEEGLDYGG LAREWFELLS HEVLNPMYCL FEYAGKNNYC LQINPASTIN | 650 |
| PDHLSYFCFI GRFIAMALFH GKFIDTGFSL PFYKRMLSKK LTIKDLESID | 700 |
| TEFYNSLIWI RDNNIEECGL EMYFSVDMEI LGKVTSHDLK LGGSNILVTE | 750 |
| ENKDEYIGLM TEWRFSRGVQ EQTKAFLDGF NEVVPLQWLQ YFDEKELEVM | 800 |
| LCGMQEVDLA DWQRNTVYRH YTRNSKQIIW FWQFVKETDN EVRMRLLQFV | 850 |
| TGTCRLPLGG FAELMGSNGP QKFCIEKVGK DTWLPRSHTC FNRLDLPPYK | 900 |
| SYEQLKEKLL FAIEETEGFG QE | 922 |

WW1 (349-382):
(SEQ ID NO: 5)
ETLPSGWEQRKDPHGRTYYVDHNTRTTTWERPQP.
WW2 (381-414):
(SEQ ID NO: 6)
QPLPPGWERRVDDRRRVYYVDHNTRTTTWORPTM.
WW3 (456-489):
(SEQ ID NO: 7)
ENDPYGPLPPGWEKRVDSTDRVYFVNHNTKTTOWEDPRT.
WW4 (496-529):
(SEQ ID NO: 8)
EPLPEGWEIRYTREGVRYFVDHNTRTTTFKDPRN.

Human WWP2 amino acid sequence (uniprot.org/uniprot/O00308). The four underlined WW domains correspond to amino acids 300-333 (WW1), 330-363 (WW2), 405-437 (WW3), and 444-547 (WW4).

| (SEQ ID NO: 9) | |
|---|---|
| MASASSSRAG VALPFEKSQL TLKVVSAKPK VHNRQPRINS YVEVAVDGLP | 50 |
| SETKKTGKRI GSSELLWNEI IILNVTAQSH LDLKVWSCHT LRNELLGTAS | 100 |
| VNLSNVLKNN GGKMENMQLT LNLQTENKGS VVSGGELTIF LDGPTVDLGN | 150 |
| VPNGSALTDG SQLPSRDSSG TAVAPENRHQ PPSTNCFGGR SRTHRHSGAS | 200 |
| ARTTPATGEQ SPGARSRHRQ PVKNSGHSGL ANGTVNDEPT TATDPEEPSV | 250 |
| VGVTSPPAAP LSVTPNPNTT SLPAPATPAE GEEPSTSGTQ QLPAAAQAPD | 300 |
| ALPAGWEQRE LPNGRVYYVD HNTKTTTWER PLPPGWEKRT DPRGRFYYVD | 350 |
| HNTRTTTWQR PTAEYVRNYE QWQSQRNQLQ GAMQHFSQRF LYQSSSASTD | 400 |
| HDPLGPLPPG WEKRQDNGRV YYVNHNTRTT QWEDPRTQGM IQEPALPPGW | 450 |
| EMKYTSEGVR YFVDHNTRTT TFKDPRPGFE SGTKQGSPGA YDRSFRWKYH | 500 |
| QFRFLCHSNA LPSHVKISVS RQTLFEDSFQ QIMNMKPYDL RRRLYIIMRG | 550 |
| EEGLDYGGIA REWFELLSHE VLNPMYCLFE YAGKNNYCLQ INPASSINPD | 600 |
| HLTYFRFIGR FIAMALYHGK FIDTGFTLPF YKRMLNKRPT LKDLESIDPE | 650 |
| FYNSIVWIKE NNLEECGLEL YFIQDMEILG KVTTHELKEG GESIRVTEEN | 700 |
| KEEYIMLLTD WRFTRGVEEQ TKAFLDGFNE VAPLEWLRYF DEKELELMLC | 750 |
| GMQEIDMSDW QKSTIYRHYT KNSKQIQWFW QVVKEMDNEK RIRLLQFVTG | 800 |
| TCRLPVGGFA ELIGSNGPQK FCIDKVGKET WLPRSHTCFN RLDLPPYKSY | 850 |
| EQLREKLLYA IEETEGFGQE | 870 |

WW1 (300-333):
(SEQ ID NO: 10)
DALPAGWEQRELPNGRVYYVDHNTKTTTWERPLP.
WW2 (330-363):
(SEQ ID NO: 11)
PLPPGWEKRTDPRGRFYYVDHNTRTTTWORPTA.
WW3 (405-437):
(SEQ ID NO: 12)
HDPLGPLPPGWEKRQDNGRVYYVNHNTRTTQWEDPRT.
WW4 (444-477):
(SEQ ID NO: 13)
PALPPGWEMKYTSEGVRYFVDHNTRTTTFKDPRP.

Human Nedd4-1 amino acid sequence (uniprot.org/uniprot/P46934). The four underlined WW domains correspond to amino acids 610-643 (WW1), 767-800 (WW2), 840-873 (WW3), and 892-925 (WW4).

| (SEQ ID NO: 14) | |
|---|---|
| MAQSLRLHFA ARRSNTYPLS ETSGDDLDSH VHMCFKRPTR ISTSNVVQMK | 50 |
| LTPRQTALAP LIKENVQSQE RSSVPSSENV NKKSSCLQIS LQPTRYSGYL | 100 |
| QSSNVLADSD DASFTCILKD GIYSSAVVDN ELNAVNDGHL VSSPAICSGS | 150 |
| LSNFSTSDNG SYSSNGSDFG SCASITSGGS YTNSVISDSS SYTFPPSDDT | 200 |
| FLGGNLPSDS TSNRSVPNRN TTPCEIFSRS TSTDPFVQDD LEHGLEIMKL | 250 |
| PVSRNTKIPL KRYSSLVIFP RSPSTTRPTS PTSLCTLLSK GSYQTSHQFI | 300 |
| ISPSEIAHNE DGTSAKGFLS TAVNGLRLSK TICTPGEVRD IRPLHRKGSL | 350 |
| QKKIVLSNNT PRQTVCEKSS EGYSCVSVHF TQRKAATLDC ETTNGDCKPE | 400 |
| MSEIKLNSDS EYIKLMHRTS ACLPSSQNVD CQININGELE RPHSQMNKNH | 450 |
| GILRRSISLG GAYPNISCLS SLKHNCSKGG PSQLLIKFAS GNEGKVDNLS | 500 |
| RDSNRDCTNE LSNSCKTRDD FLGQVDVPLY PLPTENPRLE RPYTFKDFVL | 550 |
| HPRSHKSRVK GYLRLKMTYL PKTSGSEDDN AEQAEELEPG WVVLDQPDAA | 600 |
| CHLQQQQEPS PLPPGWEERQ DILGRTYYVN HESRRTQWKR PTPQDNLTDA | 650 |
| ENGNIQLQAQ RAFTTRRQIS EETESVDNRE SSENWEIIRE DEATMYSNQA | 700 |
| FPSPPPSSNL DVPTHLAEEL NARLTIFGNS AVSQPASSSN HSSRRGSLQA | 750 |
| YTFEEQPTLP VLLPTSSGLP PGWEEKQDER GRSYYVDHNS RTTTWTKPTV | 800 |
| QATVETSQLT SSQSSAGPQS QASTSDSGQQ VTQPSEIEQG FLPKGWEVRH | 850 |
| APNGRPFFID HNTKTTTWED PRLKIPAHLR GKTSLDTSND LGPLPPGWEE | 900 |
| RTHTDGRIFY INHNIKRTQW EDPRLENVAI TGPAVPYSRD YKRKYEFFRR | 950 |
| KLKKQNDIPN KFEMKLRRAT VLEDSYRRIM GVKRADFLKA RLWIEFDGEK | 1000 |
| GLDYGGVARE WFFLISKEMF NPYYGLFEYS ATDNYTLQIN PNSGLCNEDH | 1050 |
| LSYFKFIGRV AGMAVYHGKL LDGFFIRPFY KMMLHKPITL HDMESVDSEY | 1100 |
| YNSLRWILEN DPTELDLRFI IDEELFGQTH QHELKNGGSE IVVTNKNKKE | 1150 |
| YIYLVIQWRF VNRIQKQMAA FKEGFFELIP QDLIKIFDEN ELELLMCGLG | 1200 |
| DVDVNDWREH TKYKNGYSAN HQVIQWFWKA VLMMDSEKRI RLLQFVTGTS | 1250 |
| RVPMNGFAEL YGSNGPQSFT VEQWGTPEKL PRAHTCFNRL DLPPYESFEE | 1300 |
| LWDKLQMAIE NTQGFDGVD | 1319 |

WW1(610-643):
(SEQ ID NO: 15)
SPLPPGWEERQDILGRTYYVNHESRRTQWKRPTP.
WW2(767-800):
(SEQ ID NO: 16)
SGLPPGWEEKQDERGRSYYVDHNSRTTTWTKPTV.
WW3 (840-873):
(SEQ ID NO: 17)
GFLPKGWEVRHAPNGRPFFIDHNTKTTTWEDPRL.
WW4 (892-925):
(SEQ ID NO: 18)
GPLPPGWEERTHTDGRIFYINHNIKRTQWEDPRL.

Human Nedd4-2 amino acid sequence (>gi|21361472|ref|NP_056092.2|E3 ubiquitin-protein ligase NEDD4-like isoform 3 [Homo sapiens]). The four underlined WW domains correspond to amino acids 198-224 (WW1), 368-396 (WW2), 480-510 (WW3), and 531-561 (WW4).

(SEQ ID NO: 19)
MATGLGEPVYGLSEDEGESRILRVKVVSGIDLAKKDIFGASDPYVKLSLYVADENRELALVQ
TKTIKKTLNPKWNEEFYFRVNPSNHRLLFEVFDENRLTRDDFLGQVDVPLSHLPTEDPTMER
PYTFKDFLLRPRSHKSRVKGFLRLKMAYMPKNGGQDEENSDQRDDMEHGWEVVDSNDSASQH
QEELPPPPLPPGWEEKVDNLGRTYYVNHNNRTTQWHRPSLMDVSSESDNNIRQINQEAAHRR
FRSRRHISEDLEPEPSEGGDVPEPWETISEEVNIAGDSLGLALPPPPASPGSRTSPQELSEE
LSRRLQITPDSNGEQFSSLIQREPSSRLRSCSVTDAVAEQGHLPPPSVAYVHTTPGLPSGWE
ERKDAKGRTYYVNHNNRTTTWTRPIMQLAEDGASGSATNSNNHLIEPQIRRPRSLSSPTVTL
SAPLEGAKDSPVRRAVKDTLSNPQSPQPSPYNSPKPQHKVTQSFLPPGWEMRIAPNGRPFFI
DHNTKTTTWEDPRLKFPVHMRSKTSLNPNDLGPLPPGWEERIHLDGRTFYIDHNSKITQWED
PRLQNPAITGPAVPYSREFKQKYDYFRKKLKKPADIPNRFEMKLHRNNIFEESYRRIMSVKR
PDVLKARLWIEFESEKGLDYGGVAREWFFLLSKEMENPYYGLFEYSATDNYTLQINPNSGLC
NEDHLSYFTFIGRVAGLAVFHGKLLDGFFIRPFYKMMLGKQITLNDMESVDSEYYNSLKWIL
ENDPTELDLMFCIDEENFGQTYQVDLKPNGSEIMVTNENKREYIDLVIQWRFVNRVQKQMNA
FLEGFTELLPIDLIKIFDENELELLMCGLGDVDVNDWRQHSIYKNGYCPNHPVIQWFWKAVL
LMDAEKRIRLLQFVTGTSRVPMNGFAELYGSNGPQLFTIEQWGSPEKLPRAHTCFNRLDLPP
YETFEDLREKLLMAVENAQGFEGVD
WW1 (198-224):
(SEQ ID NO: 20)
GWEEKVDNLGRTYYVNHNNRTTQWHRP.
WW2 (368-396):
(SEQ ID NO: 21)
PSGWEERKDAKGRTYYVNHNNRTTTWTRP.
WW3 (480-510):
(SEQ ID NO: 22)
PPGWEMRIAPNGRPFFIDHNTKTTTWEDPRL.
WW4 (531-561):
(SEQ ID NO: 23)
PPGWEERIHLDGRTFYIDHNSKITQWEDPRL.

Human Smurf1 amino acid sequence (uniprot.org/uniprot/Q9HCE7). The two underlined WW domains correspond to amino acids 234-267 (WW1) and 306-339 (WW2).

| (SEQ ID NO: 24) | |
|---|---|
| MSNPGTRRNG SSIKIRLTVL CAKNLAKKDF FRLPDPFAKI VVDGSGQCHS | 50 |
| TDTVKNTLDP KWNQHYDLYV GKTDSITISV WNHKKIHKKQ GAGFLGCVRL | 100 |
| LSNAISRLKD TGYQRLDLCK LNPSDTDAVR GQIVVSLQTR DRIGTGGSV | 150 |
| DCRGLLENEG TVYEDSGPGR PLSCEMEEPA PYTDSTGAAA GGGNCRFVES | 200 |
| PSQDQRLQAQ RLRNPDVRGS LQTPQNRPHG HQSPELPEGY EQRTTVQGQV | 250 |
| YFLHTQTGVS TWHDPRIPSP SGTIPGGDAA FLYEFLLQGH TSEPRDLNSV | 300 |
| NCDELGPLPP GWEVRSTVSG RIYFVDHNNR TTQFTDPRLH HIMNHQCQLK | 350 |
| EPSQPLPLPS EGSLEDEELP AQRYERDLVQ KLKVLRHELS LQQPQAGHCR | 400 |
| IEVSREEIFE ESYRQIMKMR PKDLKKRLMV KFRGEEGLDY GGVAREWLYL | 450 |
| LCHEMLNPYY GLFQYSTDNI YMLQINPDSS INPDHLSYFH FVGRIMGLAV | 500 |
| FHGHYINGGF TVPFYKQLLG KPIQLSDLES VDPELHKSLV WILENDITPV | 550 |
| LDHTFCVEHN AFGRILQHEL KPNGRNVPVT EENKKEYVRL YVNWRFMRGI | 600 |
| EAQFLALQKG FNELIPQHLL KPFDQKELEL IIGGLDKIDL NDWKSNTRLK | 650 |
| HCVADSNIVR WEWQAVETFD EERRARLLQF VTGSTRVPLQ GFKALQGSTG | 700 |
| AAGPRLFTIH LIDANTDNLP KAHTCENRID IPPYESYEKL YEKLLTAVEE | 750 |
| TCGFAVE | 757 |

WW1 (234-267):
(SEQ ID NO: 25)
PELPEGYEQRTTVQGQVYFLHTQTGVSTWHDPRI.
WW2 (306-339):
(SEQ ID NO: 26)
GPLPPGWEVRSTVSGRIYFVDHNNRTTQFTDPRL.

Human Smurf2 amino acid sequence (uniprot.org/uniprot/Q9HAU4). The three underlined WW domains correspond to amino acids 157-190 (WW1), 251-284 (WW2), and 297-330 (WW3).

| (SEQ ID NO: 27) | |
|---|---|
| MSNPGGRRNG PVKLRLTVLC AKNLVKKDFF RLPDPFAKVV VDGSGQCHST | 50 |
| DTVKNTLDPK WNQHYDLYIG KSDSVTISVW NHKKIHKKQG AGFLGCVRLL | 100 |
| SNAINRLKDT GYQRLDLCKL GPNDNDTVRG QIVVSLQSRD RIGTGGQVVD | 150 |
| CSRLFDNDLP DGWEERRTAS GRIQYLNHIT RTTQWERPTR PASEYSSPGR | 200 |
| PLSCFVDENT PISGINGATC GQSSDPRLAE RRVRSQRHRN YMSRTHLHTP | 250 |
| PDLPEGYEQR TTQQGQVYFL HTQTGVSTWH DPRVPRDLSN INCEELGPLP | 300 |
| PGWEIRNTAT GRVYFVDHNN RTTQFTDPRL SANLHLVLNR QNQLKDQQQQ | 350 |
| QVVSLCPDDT ECLTVPRYKR DLVQKLKILR QELSQQQPQA GHCRIEVSRE | 400 |
| EIFEESYRQV MKMRPKDLWK RLMIKFRGEE GLDYGGVARE WLYLLSHEML | 450 |
| NPYYGLFQYS RDDIYTLQIN PDSAVNPEHL SYFHFVGRIM GMAVFHGHYI | 500 |
| DGGFTLPFYK QLLGKSITLD DMELVDPDLH NSLVWILEND ITGVLDHTFC | 550 |
| VEHNAYGEII QHELKPNGKS IPVNEENKKE YVRLYVNWRF LRGIEAQFLA | 600 |
| LQKGFNEVIP QHLLKTFDEK ELELIICGLG KIDVNDWKVN TRLKHCTPDS | 650 |
| NIVKWFWKAV EFFDEERRAR LLQFVTGSSR VPLQGFKALQ GAAGPRLFTI | 700 |
| HQIDACTNNL PKAHTCFNRI DIPPYESYEK LYEKLLTAIE ETCGFAVE | 748 |

WW1 (157-190):
(SEQ ID NO: 28)
NDLPDGWEERRTASGRIQYLNHITRTTQWERPTR.
WW2 (251-284):
(SEQ ID NO: 29)
PDLPEGYEQRTTQQGQVYFLHTQTGVSTWHDPRV.
WW3 (297-330):
(SEQ ID NO: 30)
GPLPPGWEIRNTATGRVYFVDHNNRTTQFTDPRL.

Human ITCH amino acid sequence (uniprot.org/uniprot/Q96J02). The four underlined WW domains correspond to amino acids 326-359 (WW1), 358-391 (WW2), 438-471 (WW3), and 478-511 (WW4).

| (SEQ ID NO: 31) | |
|---|---|
| MSDSGSQLGS MGSLTMKSQL QITVISAKLK ENKKNWFGPS PYVEVTVDGQ | 50 |
| SKKTEKCNNT NSPKWKQPLT VIVTPVSKLH FRVWSHQTLK SDVLLGTAAL | 100 |
| DIYETLKSNN MKLEEVVVTL QLGGDKEPTE TIGDLSICLD GLQLESEVVT | 150 |
| NGETTCSENG VSLCLPRLEC NSAISAHCNL CLPGLSDSPI SASRVAGFTG | 200 |
| ASQNDDGSRS KDETRVSTNG SDDPEDAGAG ENRRVSGNNS PSLSNGGFKP | 250 |
| SRPPRPSRPP PPTPRRPASV NGSPSATSES DGSSTGSLPP TNTNTNTSEG | 300 |
| ATSGLIIPLT ISGGSGPRPL NPVTQAPLPP GWEQRVDQHG RVYYVDHVEK | 350 |
| RTTWDRPEPL PPGWFRRVDN MGRIYYVDHF TRTTTWQRPT LESVRNYEQW | 400 |
| QLQRSQLQGA MQQFNQRFIY GNQDLFATSQ SKEFDPLGPL PPGWEKRTDS | 450 |
| NGRVYFVNHN TRITQWEDPR SQGQLNEKPL PEGWEMRFTV DGIPYFVDHN | 500 |
| RRTTTYIDPR TGKSALDNGP QIAYVRDFKA KVQYFRFWCQ QLAMPQHIKI | 550 |
| TVTRKTLFED SFQQIMSFSP QDLRRRLWVI FPGEEGLDYG GVAREWFFLL | 600 |
| SHEVLNPMYC LFEYAGKDNY CLQINPASYI NPDHLKYFRF IGRFIAMALE | 650 |
| HGKFIDTGFS LPFYKRILNK PVGLKDLESI DPEFYNSLIW VKENNIEECD | 700 |
| LEMYFSVDKE ILGEIKSHDL KPNGGNILVT EENKEEYIRM VAEWRLSRGV | 750 |
| EEQTQAFFEG FNEILPQQYL QYFDAKELEV LLCGMQEIDL NDWQRHAIYR | 800 |
| HYARTSKQIM WFWQFVKEID NEKRMRLLQF VTGTCRLPVG GFADLMGSNG | 850 |
| PQKFCIEKVG KENWLPRSHT CFNRLDLPPY KSYEQLKEKL LFAIEETEGE | 900 |
| GQE | 903 |

ITCH WW1 (326-359):
(SEQ ID NO: 32)
APLPPGWEQRVDQHGRVYYVDHVEKRTTWDRPEP.
ITCH WW2 (358-391):
(SEQ ID NO: 33)
EPLPPGWERRVDNMGRIYYVDHFTRITTWQRPTL.
ITCH WW3 (438-471):
(SEQ ID NO: 34)
GPLPPGWEKRTDSNGRVYFVNHNTRITQWEDPRS.
ITCH WW4 (478-511):
(SEQ ID NO: 35)
KPLPEGWEMRFTVDGIPYFVDHNRRTTTYIDPRT.

Human NEDL1 amino acid sequence (uniprot.org/uniprot/Q76N89). The two underlined WW domains correspond to amino acids 829-862 (WW1), and 1018-1051 (WW2).

| (SEQ ID NO: 36) | |
|---|---|
| MLLHLCSVKN LYQNRFLGLA AMASPSRNSQ SRRRCKEPLR YSYNPDQFHN | 50 |
| MDLRGGPHDG VTIPRSTSDT DLVTSDSRST LMVSSSYYSI GHSQDLVIHW | 100 |
| DIKEEVDAGD WIGMYLIDEV LSENFLDYKN RGVNGSHRGQ IIWKIDASSY | 150 |
| FVEPETKICF KYYHGVSGAL RATTPSVTVK NSAAPIFKSI GADETVQGQG | 200 |
| SRRLISESLS DFQAMGLKKG MFFNPDPYLK ISIQPGKHSI FPALPHHGQE | 250 |
| RRSKIIGNTV NPIWQAEQFS FVSLPTDVLE IEVKDKFAKS RPIIKRFLGK | 300 |
| LSMPVQRLLE RHAIGDRVVS YTLGRRLPTD HVSGQLQFRF EITSSIHPDD | 350 |
| EEISLSTEPE SAQIQDSPMN NLMESGSGEP RSEAPESSES WKPEQLGEGS | 400 |
| VPDGPGNQSI ELSRPAEEAA VITEAGDQGM VSVGPEGAGE LLAQVQKDIQ | 450 |
| PAPSAEELAE QLDLGEEASA LLLEDGEAPA STKEEPLEEE ATTQSRAGRE | 500 |
| EEEKEQEEEG DVSTLEQGEG RLQLRASVKR KSRPCSLPVS ELETVIASAC | 550 |
| GDPETPRTHY IRIHTLLHSM PSAQGGSAAE EEDGAEEEST LKDSSEKDGL | 600 |
| SEVDTVAADP SALEEDREEP EGATPGTAHP GHSGGHFPSL ANGAAQDGDT | 650 |
| HPSTGSESDS SPRQGGDHSC EGCDASCCSP SCYSSSCYST SCYSSSCYSA | 700 |
| SCYSPSCYNG NRFASHTRES SVDSAKISES TVFSSQDDEE EENSAFESVP | 750 |
| DSMQSPELDP ESTNGAGPWQ DELAAPSGHV ERSPEGLESP VAGPSNRREG | 800 |
| ECPILHNSQP VSQLPSLRPE HHHYPTIDEP LPPNWEARID SHGRVFYVDH | 850 |
| VNRTTTWQRP TAAATPDGMR RSGSIQQMEQ LNRRYQNIQR TIATERSEED | 900 |
| SGSQSCEQAP AGGGGGGGSD SEAESSQSSL DLRREGSLSP VNSQKITLLL | 950 |
| QSPAVKFITN PEFFTVLHAN YSAYRVFTSS TCLKHMILKV RRDARNFERY | 1000 |
| QHNRDLVNFI NMFADTRLEL PRGWEIKTDQ QGKSFFVDHN SRATTFIDPR | 1050 |
| IPLQNGRLPN HLTHRQHLQR LRSYSAGEAS EVSRNRGASL LARPGHSLVA | 1100 |
| AIRSQHQHES LPLAYNDKIV AFLRQPNIFE MLQERQPSLA RNHTLREKIH | 1150 |
| YIRTEGNHGL EKLSCDADLV ILLSLFEEEI MSYVPLQAAF HPGYSFSPRC | 1200 |
| SPCSSPQNSP GLQRASARAP SPYRRDFEAK LRNFYRKLEA KGFGQGPGKI | 1250 |
| KLIIRRDHLL EGTFNQVMAY SRKELQRNKL YVTFVGEEGL DYSGPSREFF | 1300 |
| FLLSQELFNP YYGLFEYSAN DTYTVQISPM SAFVENHLEW FRESGRILGL | 1350 |
| ALIHQYLLDA FFTRPFYKAL LRLPCDLSDL EYLDEEFHQS LQWMKDNNIT | 1400 |
| DILDLTFTVN EEVFGQVTER ELKSGGANTQ VTEKNKKEYI ERMVKWRVER | 1450 |
| GVVQQTEALV RGFYEVVDSR LVSVFDAREL ELVIAGTAEI DLNDWRNNTE | 1500 |
| YRGGYHDGHL VIRWFWAAVE RENNEQRLRL LQFVTGTSSV PYEGFAALRG | 1550 |
| SNGLRRFCIE KWGKITSLPR AHTCFNRLDL PPYPSYSMLY EKLLTAVEET | 1600 |
| STFGLE | 1606 |

WW1 (829-862):
(SEQ ID NO: 37)
PLPPNWEARIDSHGRVFYVDHVNRTTTWQRPTA.
WW2 (1018-1051):
(SEQ ID NO: 38)
LELPRGWEIKTDQQGKSFFVDHNSRATTFIDPRI.

Human NEDL2 amino acid sequence (uniprot.org/uniprot/Q9P2P5). The two underlined WW domains correspond to amino acids 807-840 (WW1) and 985-1018 (WW2).

| (SEQ ID NO: 39) | |
|---|---|
| MASSAREHLL FVRRRNPQMR YTLSPENLQS LAAQSSMPEN MTLQRANSDT | 50 |
| DLVTSESRSS LTASMYEYTL GQAQNLIIFW DIKEEVDPSD WIGLYHIDEN | 100 |
| SPANFWDSKN RGVTGTQKGQ IVWRIEPGPY FMEPEIKICF KYYHGISGAL | 150 |
| RATTPCITVK NPAVMMGAEG MEGGASGNLH SRKLVSFTLS DLRAVGLKKG | 200 |
| MFFNPDPYLK MSIQPGKKSS FPTCAHHGQE RRSTIISNTT NPIWHREKYS | 250 |
| FFALLTDVLE IEIKDKFAKS RPIIKRFLGK LTIPVQRLLE RQAIGDQMLS | 300 |
| YNLGRRLPAD HVSGYLQFKV EVTSSVHEDA SPEAVGTILG VNSVNGDLGS | 350 |
| PSDDEDMPGS HHDSQVCSNG PVSEDSAADG TPKHSFRTSS TLEIDTEELT | 400 |
| STSSRTSPPR GRQDSLNDYL DAIEHNGHSR PGTATCSERS MGASPKLRSS | 450 |
| FPTDTRLNAM LHIDSDEEDH EFQQDLGYPS SLEEEGGLIM FSRASRADDG | 500 |
| SLTSQTKLED NPVENEEAST HEAASFEDKP ENLPELAESS LPAGPAPEEG | 550 |
| EGGPEPQPSA DQGSAELCGS QEVDQPTSGA DTGTSDASGG SRRAVSETES | 600 |
| LDQGSEPSQV SSETEPSDPA RTESVSEAST RPEGESDLEC ADSSCNESVT | 650 |
| TQLSSVDTRC SSLESARFPE TPAFSSQEEE DGACAAEPTS SGPAEGSQES | 700 |
| VCTAGSLPVV QVPSGEDEGP GAESATVPDQ EELGEVWQRR GSLEGAAAAA | 750 |
| ESPPQEEGSA GEAQGTCEGA TAQEEGATGG SQANGHQPLR SLPSVRQDVS | 800 |
| RYQRVDEALP PNWEARIDSH GRIFYVDHVN RTTTWQRPTA PPAPQVLQRS | 850 |
| NSIQQMEQLN RRYQSIRRTM TNERPEENTN AIDGAGEEAD FHQASADFRR | 900 |
| ENILPHSTSR SRITLLLQSP PVKFLISPEF FTVLHSNPSA YRMFTNNTCL | 950 |
| KHMITKVRRD THHFERYQHN RDLVGFLNMF ANKQLELPRG WEMKHDHQGK | 1000 |
| AFFVDHNSRT TTFIDPRLPL QSSRPTSALV HRQHLTRQRS HSAGEVGEDS | 1050 |
| RHAGPPVLPR PSSTFNTVSR PQYQDMVPVA YNDKIVAFLR QPNIFEILQE | 1100 |
| RQPDLTRNHS LREKIQFIRT EGTPGLVRLS SDADLVMLLS LFEEEIMSYV | 1150 |
| PPHALLHPSY CQSPRGSPVS SPQNSPGTQR ANARAPAPYK RDFEAKLRNF | 1200 |
| YRKLETKGYG QGPGKLKLII RRDHLLEDAF NQIMGYSRKD LQRNKLYVTF | 1250 |
| VGEEGLDYSG PSREFFFLVS RELFNPYYGL FEYSANDTYT VQISPMSAFV | 1300 |
| DNHHEWFRFS GRILGLALIH QYLLDAFFTR PFYKALLRIL CDLSDLEYLD | 1350 |
| EEFHQSLQWM KDNDIHDILD LTFTVNEEVF GQITERELKP GGANIPVTEK | 1400 |
| NKKEYIERMV KWRIERGVVQ QTESLVRGFY EVVDARLVSV FDARELELVI | 1450 |
| AGTAEIDLSD WRNNTEYRGG YHDNHIVIRW FWAAVERENN EQRLRLLQFV | 1500 |
| TGTSSIPYEG FASLRGSNGP RRECVEKWGK ITALPRAHTC FNRLDLPPYP | 1550 |
| SFSMLYEKLL TAVEETSTEG LE | 1572 |

WW1 (807-840):
(SEQ ID NO: 40)
EALPPNWEARIDSHGRIFYVDHVNRTTTWORPTA.
WW2 (985-1018):
(SEQ ID NO: 41)
LELPRGWEMKHDHQGKAFFVDHNSRTTTFIDPRL.

In some embodiments, the WW domain consists essentially of a WW domain or WW domain variant. Consists essentially of means that a domain, peptide, or polypeptide consists essentially of an amino acid sequence when such an amino acid sequence is present with only a few additional amino acid residues, for example, from about 1 to about 10 or so additional residues, typically from 1 to about 5 additional residues in the domain, peptide, or polypeptide.

Alternatively, the WW domain may be a WW domain that has been modified to include two basic amino acids at the C-terminus of the domain. Techniques are known in the art and are described in the art, for example, in Sambrook et al., ((2001) Molecular Cloning: a Laboratory Manual, 3rd ed., Cold Spring Harbour Laboratory Press). Thus, a skilled person could readily modify an existing WW domain that does not normally have two C-terminal basic residues so as to include two basic residues at the C-terminus.

Basic amino acids are amino acids that possess a side-chain functional group that has a pKa of greater than 7 and includes lysine, arginine, and histidine, as well as basic amino acids that are not included in the twenty α-amino acids commonly included in proteins. The two basic amino acids at the C-terminus of the WW domain may be the same basic amino acid or may be different basic amino acids. In one embodiment, the two basic amino acids are two arginines.

The term WW domain also includes variants of a WW domain provided that any such variant possesses two basic amino acids at its C-terminus and maintains the ability of the WW domain to associate with the PPXY (SEQ ID NO: 2) motif. A variant of such a WW domain refers to a WW domain which retains the ability of the variant to associate with the PPXY (SEQ ID NO: 2) motif (i.e., the PPXY (SEQ ID NO:2) motif of ARRDC1 and that has been mutated at one or more amino acids, including point, insertion, and/or deletion mutations, but still retains the ability to associate with the PPXY (SEQ ID NO: 2) motif. A variant or derivative therefore includes deletions, including truncations and fragments; insertions and additions, for example, conservative substitutions, site-directed mutants and allelic variants; and modifications, including one or more non-amino acyl groups (e.g., sugar, lipid, etc.) covalently linked to the peptide and post-translational modifications. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

The WW domain may be part of a longer protein. Thus, the protein, in various different embodiments, comprises the WW domain, consists of the WW domain, or consists essentially of the WW domain, as defined herein. The polypeptide may be a protein that includes a WW domain as a functional domain within the protein sequence.

### GENERAL DESCRIPTION OF THE INVENTION

Representative Diseases Associated with Schwann Cells

### 1. CMT Diseases

The CMT diseases are a group of genetically determined disorders that are influenced by nearly 100 genes. There are many different types of CMT disease, which may share various symptoms, but vary by the pattern of inheritance, age of onset, and extent of involvement of the axon or myelin sheath. Concerning the age of onset, the various CMTs have been characterized as neonatal or “congenital,” infantile, or late-onset. These ages of onset are often further categorized as early infantile (<2 years), childhood (~2 to 10 years), juvenile (~10 to 20 years), adult (~20 to 50 years), and late adult (>50 years) onset. (Rudnik-Schönebom, S., et al., “Diagnostic algorithms in Charcot-Marie-Tooth neuropathies: experiences from a German genetic laboratory on the basis of 1206 index patients,” Clin. Genet., 89(1):34-43 (2016)).

The CMT diseases are presently classified based on electrophysiological findings as either demyelinating or axonal neuropathies, although new and additional genetic tests for the disease are becoming available. Various CMTs show autosomal dominant (i.e., CMT1A), autosomal recessive, or X-linked patterns of inheritance. The autosomal dominant pattern of inheritance is the most common presentation. (Tazir, M., et al., “Hereditary motor and sensory neuropathies or Charcot-Marie-Tooth diseases: an update,” J. Neurol. Sci., 15; 347(1-2):14-22 (2014)). The majority of CMT occurs as the CMT1A forms of the disease.

In CMT diseases, as much as 90% of observed genetic abnormalities are due to copy number variations (e.g., increase in copy number) in the PMP22 gene and, to a lesser extent, mutations in the GJB1, MPZ, and MFN2 genes. The frequency of abnormalities in other genes individually is rare. (See, Gess, B., et al., “Charcot-Marie-Tooth disease: frequency of genetic subtypes in a German neuromuscular center population,” Neuromuscul. Disord., 23(8):647-651 (2013); and Milley, G. M., et al., “Genotypic and phenotypic spectrum of the most common causative genes of Charcot-Marie-Tooth disease in Hungarian patients,” Neuromuscul. Disord., 28(1):38-43 (2018)). Copy number variation in the PMP22 gene is the most common cause of CMT1A. The PMP22 gene encodes a protein called the peripheral myelin protein-22; the gene is in the middle of the 1.4 Mb regions in chromosome17p.12. Duplication or deletion mutations in PMP22 are thought to lead to disease via “gene dosage” effects. (See, Cutrupi, A N., et al., “Structural variations causing inherited peripheral neuropathies: A paradigm for understanding genomic organization, chromatin interactions, and gene dysregulation,” Mol. Genet. Genomic Med., 6(3):422-433 (2018)). Duplication in the heterozygous state results in 1.5-fold overexpression, while homozygous duplication causes two-fold overexpression of PMP22 in Schwann cells. This genomic duplication results from an unequal meiotic crossover facilitated in the male germline by the flanking homologous repeat sequences. PMP22 overexpression is believed to overload the proteasome system and leads to cytoplasmic aggregation of the ubiquitinated PMP22 protein. The aggregation of pmp22 protein causes the recruitment of auto-phagosomes and lysosomes and increased autophagy. (See, Fortun, J., et al., “Impaired proteasome activity and accumulation of ubiquitinated substrates in a hereditary neuropathy model,” J. Neurochem., 92(6):1531-1541 (2005)). Other CMTs are thought to result from loss of function mutations in other genes or, uncommonly, due to toxic gain of function mutations.

In certain embodiments, the present invention provides compositions and methods of using these compositions to treat diseases and pathologies and deleterious conditions in cells of the peripheral nervous system, particularly, Schwann cells.

### 2. Schwannomas

Schwannomas are benign, solitary and sporadic (in 90% of cases), well-encapsulated, slow-growing nerve sheath tumors of Schwann cells. These tumors can originate from any myelinated central or peripheral nerve with Schwann cells. Schwannoma is the most common type of nerve sheath tumor, occurring in approximately 89% of the cases of nerve sheath tumors. Schwannoma accounts for significant morbidity in the U.S. adults, the median age at diagnosis is 56 years, with from 4.4 to 5.23 cases per 100,000 adults/year reported. (See, Ostrom, Q. T., et al., “CBTRUS Statistical Report: Primary Brain and Other Central Nervous System Tumors Diagnosed in the United States in 2012-2016,” Neuro. Oncol., 21(Suppl 5):v 1-v 100 (2019)).

Schwannomas grow slowly and may be present for years before being noticed due to long periods before becoming symptomatic. Schwannomas can occur in various locations, such as the upper limbs, head, inner/idle ear, trunk, flexor surfaces of the lower extremities, posterior mediastinum, retroperitoneum, spinal roots, bone, gastrointestinal tract, pancreas, liver, thyroid, adrenal glands, and lymph nodes) with corresponding varied clinical presentations. Schwannomas in the extremities may occur as asymptomatic masses that cause mild local pain and paresthesia due to pressure on parent nerve cells. In more severe manifestations, sciatic nerve schwannomas can mimic disk herniation and cause severe low back pain with radiation down the leg. Thoracic outlet syndrome can result from schwannoma involving the C7 nerve root. Schwannomas in the ankle can mimic tarsal tunnel syndrome, while those in the wrist can present as carpal tunnel syndrome.

Schwannomas that develop in the balance and hearing nerves of the inner ear are termed “vestibular.” Vestibular schwannoma can cause unilateral hearing loss, tinnitus, dizziness, loss of balance, and, rarely, facial nerve palsy. Advanced vestibular schwannomas can press against nearby brain structures and become life-threatening. Vestibular schwannomas comprise about 60% of all benign schwannomas.

The NF2 gene found on chromosome 22, which encodes the merlin protein, plays an essential role in sporadic and syndromic schwannoma development. Merlin protein is cytoskeletal and is a known tumor suppressor protein involved in neurofibromatosis type II. Sequence data show that merlin is similar to members of the ERM protein family. Specific mutations in the NF2 gene cause the inactivation of the gene and prevents the formation of merlin protein. Inactivation of both alleles of the NF2 gene is observed in most schwannomas.

In certain embodiments, the present invention provides delivery platforms, ARMMs-mediated delivery vesicles, optimized to deliver a therapeutic agent to diseased or aberrant Schwann cells.

In certain embodiments, the present invention provides delivery platforms, ARMMs-mediated delivery vesicles, optimized to deliver more than one type, or more than one representative member of a particular class or type, of therapeutic agent to diseased or aberrant Schwann cells.

In some of the embodiments, the Schwann cell related disease is a CMT, and more particularly, CMT1A.

In other embodiments, the Schwann cell related disease is a schwannoma tumor.

In some embodiments, treatments are contemplated wherein one therapeutic agent is administered using ARMM-mediated delivery of an agent to target cells, tissues, systems, or mammalian subjects.

In some other embodiments, treatments are contemplated wherein two therapeutic agents are administered using ARMM-mediated delivery of the respective agents to target cells, tissues, systems, or mammalian subjects.

In still some other embodiments, treatments are contemplated wherein three or more therapeutic agents are administered using ARMM-mediated delivery of the respective agents to target cells, tissues, systems, or mammalian subjects.

It is contemplated that within the compositions and methods described herein, when two or more respective therapeutic agents are administered (e.g., co-administered) to a target, cell, tissue, system, or subject that the respective (i.e., two or more) agents are provided in a single ARRDC1-mediated microvesicle (ARMM) particle for administration.

It is likewise further contemplated that within the compositions and methods described herein, when two or more respective therapeutic agents are administered (e.g., concomitantly administered) to a target cell, tissue, system, or subject that the respective (i.e., two or more) agents are provided respectively in different ARRDC1-mediated microvesicles (ARMMs) particles for administration.

### DETAILED DESCRIPTION OF CERTAIN EMBODIMENTS OF THE INVENTION

The present invention provides methods, systems, compositions, and strategies for the use of ARMM-mediated delivery of molecules (e.g., biological molecules, small molecules, proteins, and nucleic acids (e.g., DNA, RNA), DNA plasmids, siRNA, shRNA, mRNA, and the like), to cells of the nervous system (e.g., peripheral nervous system).

In particular, the present invention generally relates to compositions and methods of producing, testing, and administering ARRDC1-mediated microvesicles (“ARMMs”) to peripheral nervous system cells in mammalian subjects. More particularly, the present invention provides compositions and methods of producing, testing, and administering ARMMs particles comprising one or more therapeutic agents (e.g., biological molecules, small molecules, proteins, and nucleic acids (e.g., DNA, RNA), DNA plasmids, siRNA, shRNA, mRNA, and the like). Also provided are methods of administering therapeutic agents, including, but not limited to, treating, or contacting cells, tissues, and systems in one or more treatment environments (e.g., in vitro, in vivo, or ex vivo) with the invention compositions. In particular, the present invention provides methods of administering therapeutic agents via ARMMs to Schwann cells in mammalian subjects, including, but not limited to, humans. Additionally, the present invention relates to methods of creating, using, and harvesting the inventive compositions from producer cells and producer cell cultures.

The instant disclosure relates, at least in part, to the discovery that an shRNA encoding payload RNA associated with an ARRDC1 protein can be loaded into an ARMM and delivered to Schwann cells. In some embodiments, the uptake of these ARMMs and payload is enhanced by the presence of viral envelope proteins, including, but not limited to, VSV-G. Different payload types, such as payload proteins and payload nucleic acids, including payload RNA, can be loaded in such ARMMs for delivery to Schwann cells. Various types of payload proteins, payload nucleic acids, payload RNAs, payload protein, payload nucleic acid, and payload RNA are known in the art and include those described in U.S. Pat. Nos. 9,737,480; 9,816,080; 10,260,055; and PCT Application Publication WO2018/067546; the entire contents of each of which are hereby incorporated by reference in their entirety.

### ARMMs

Arrestin domain containing protein 1 mediated microvesicles (ARMMs) are extracellular vesicles (EVs) that are distinct from exosomes. The budding of ARMMs requires Arrestin domain containing protein 1 (ARRDC1), which is localized to the cytosolic side of the plasma membrane and, through a tetrapeptide motif, recruits the ESCRT-I complex protein TSG101 to the cell surface to initiate the outward membrane budding. Thus, in contrast to exosomes, the biogenesis of ARMMs occurs at the plasma membrane. ARMMs exhibit several additional features that make them potentially ideal vehicles for therapeutic delivery. ARRDC1 is not only necessary but also sufficient to drive ARMMs budding. Indeed, simple overexpression of the ARRDC1 protein increases the production of ARMMs in cells. This allows controlled production of ARMMs using modern biological manufacturing methods. Moreover, endogenous proteins such as cell surface receptors are actively recruited into ARMMs and can be delivered into recipient cells to initiate intercellular communication, suggesting that the exogenous payload molecules may be similarly packaged and delivered via ARMMs.

### ARRDC1

ARRDC1 is a protein that comprises a PSAP (SEQ ID NO: 1) motif and a PPXY (SEQ ID NO: 2) motif in its C-terminus, and interacts with TSG101 as shown herein. It should be appreciated that the PSAP (SEQ ID NO: 1) motif and the PPXY (SEQ ID NO: 2) motif are not required to be at the absolute C-terminal end of the ARRDC1. Rather, they may be at a C-terminal portion of the ARRDC1 protein (e.g., the C-terminal half of the ARRDC1). The disclosure also contemplates variants of ARRDC1, such as fragments of ARRDC1 and/or ARRDC1 proteins that have a degree of identity (e.g., 60%, 70%, 80%, 85%, 90%, 95%, 98%, or 99% identity) to an ARRDC1 protein and are capable if interacting with TSG101. Accordingly, an ARRDC1 protein may be a protein that comprises a PSAP (SEQ ID NO: 1) motif and a PPXY (SEQ ID NO: 2) motif, and interacts with TSG101. In some embodiments, the ARRDC1 protein is at least 60%, 65%, 70%, 75%, 80%, 85%, 90%, 95%, 98%, or 99% identical to the amino acid sequence of any one of SEQ ID NOs: 42-44, comprises a PSAP (SEQ ID NO: 1) motif and a PPXY (SEQ ID NO: 2) motif, and interacts with TSG101. In some embodiments, the ARRDC1 protein has at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, or at least 430 identical contiguous amino acids of any one of SEQ ID NOs: 42-44, comprises a PSAP (SEQ ID NO: 1) motif and a PPXY (SEQ ID NO: 2) motif, and interacts with TSG101. In some embodiments, the ARRDC1 protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 42-44 comprises a PSAP (SEQ ID NO: 1) motif and a PPXY (SEQ ID NO: 2) motif, and interacts with TSG101. In some embodiments, the ARRDC1 protein comprises any one of the amino acid sequences set forth in SEQ ID NOs: 42-44. Exemplary, non-limiting ARRDC1 protein sequences are provided herein, and additional, suitable ARRDC1 protein variants according to aspects of this invention are known in the art. It will be appreciated by those of skill in the art that this invention is not limited in this respect. Exemplary ARRDC1 sequences include the following (PSAP (SEQ ID NO: 1) and PPXY (SEQ ID NO: 2) motifs are marked):

>gi|22748653|ref|NP_689498.1|arrestin domain-containing protein 1 [Homo sapiens]

(SEQ ID NO: 42)
MGRVQLFEISLSHGRVVYSPGEPLAGTVRVRLGAPLPFRAIRVTCIGSCGVSNKANDTAWVV
EEGYFNSSLSLADKGSLPAGEHSFPFQFLLPATAPTSFEGPFGKIVHQVRAAIHTPRESKDH
KCSLVFYILSPLNLNSIPDIEQPNVASATKKFSYKLVKTGSVVLTASTDLRGYVVGQALQLH
ADVENQSGKDTSPVVASLLQKVSYKAKRWIHDVRTIAEVEGAGVKAWRRAQWHEQILVPALP

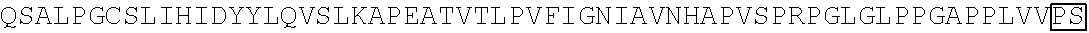

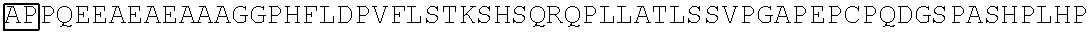

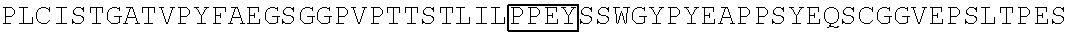

>gi|244798004|ref|NP_001155957.1|arrestin domain-containing protein 1 isoform a [Mus musculus]

(SEQ ID NO: 43)
MGRVQLFEIRLSQGRVVYGPGEPLAGTVHLRLGAPLPFRAIRVTCMGSCGVSTKANDGAWVV
EESYENSSLSLADKGSLPAGEHNFPFQFLLPATAPTSFEGPFGKIVHQVRASIDTPRESKDH
KCSLVFYILSPLNLNSIPDIEQPNVASTTKKESYKLVKTGNVVLTASTDLRGYVVGQVLRLQ
ADIENQSGKDTSPVVASLLQKVSYKAKRWIYDVRTIAEVEGTGVKAWRRAQWQEQILVPALP

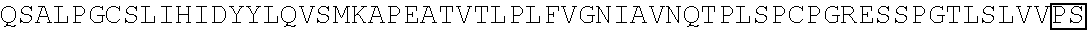

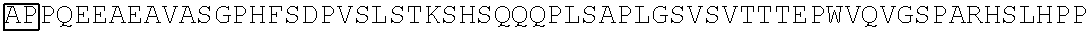

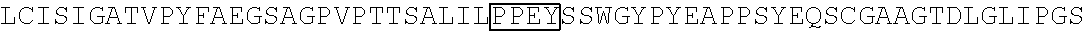

>gi|244798112|ref|NP_848495.2|arrestin domain-containing protein 1 isoform b [Mus musculus]

(SEQ ID NO: 44)
MGRVQLFEIRLSQGRVVYGPGEPLAGTVHLRLGAPLPFRAIRVTCMGSCGVSTKANDGAWVV
EESYENSSLSLADKGSLPAGEHNFPFQFLLPATAPTSFEGPFGKIVHQVRASIDTPRESKDH
KCSLVFYILSPLNLNSIPDIEQPNVASTTKKFSYKLVKTGNVVLTASTDLRGYVVGQVLRLQ
ADIENQSGKDTSPVVASLLQVSYKAKRWIYDVRTIAEVEGTGVKAWRRAQWQEQILVPALPQ

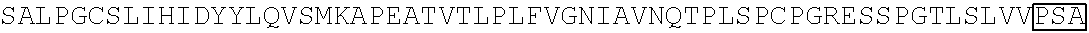

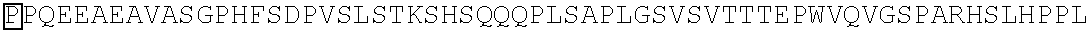

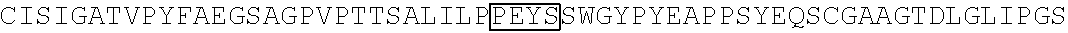

### TSG101

In certain embodiments, the inventive microvesicles further comprise TSG101 (tumor susceptibility gene 101) TSG101 belongs to a group of apparently inactive homologs of ubiquitin-conjugating enzymes. The protein contains a coiled-coil domain that interacts with stathmin, a cytosolic phosphoprotein implicated in tumorigenesis. TSG101 is a protein that comprises a UEV domain, and interacts with ARRDC1. As referred to herein, UEV refers to the Ubiquitin E2 variant domain of approximately 145 amino acids. The structure of the domain contains a α/β fold similar to the canonical E2 enzyme but has an additional N-terminal helix and further lacks the two C-terminal helices. Often found in TSG101/Vps23 proteins, the UEV interacts with a ubiquitin molecule and is essential for the trafficking of a number of ubiquitylated payloads to multivesicular bodies (MVBs). Furthermore, the UEV domain can bind to Pro-Thr/Ser-Ala-Pro peptide ligands, a fact exploited by viruses such as HIV. Thus, the TSG101 UEV domain binds to the PTAP tetrapeptide motif in the viral Gag protein that is involved in viral budding. The disclosure also contemplates variants of TSG101, such as fragments of TSG101 and/or TSG101 proteins that have a degree of identity (e.g., 60%, 70%, 80%, 85%, 90%, 95%, 98%, or 99% identity) to a TSG101 protein and are capable of interacting with ARRDC1. Accordingly, a TSG101 protein may be a protein that comprises a UEV domain, and interacts with ARRDC1. In some embodiments, the TSG101 protein is at least 60%, 65%, 70%, 75%, 80%, 85%, 90%, 95%, 98%, or 99% identical to the amino acid sequence of any one of SEQ ID NOs: 45-47, comprises a UEV domain, and interacts with ARRDC1. In some embodiments, the TSG101 protein has at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, or at least 390, identical contiguous amino acids of any one of SEQ ID NOs: 45-47, comprises a UEV domain and interacts with ARRDC1. In some embodiments, the TSG101 protein has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NOs: 45-47 and comprises a UEV domain. In some embodiments, the ARRDC1 protein comprises any one of the amino acid sequences set forth in SEQ ID NOs: 45-47. Exemplary, non-limiting TSG101 protein sequences are provided herein, and additional, suitable TSG101 protein sequences, isoforms, and variants are known in the art. It will be appreciated by those of skill in the art that this invention is not limited in this respect. Exemplary TSG101 sequences include the following sequences (the UEV domain in these sequences includes amino acids 1-145 and is underlined in the sequences below):

>gi|5454140|ref|NP_006283.1|tumor susceptibility gene 101 protein [Homo sapiens]

(SEQ ID NO: 45)
MAVSESQLKKMVSKYKYRDLTVRETVNVITLYKDLKPVLDSYVFNDGSSRELMNLTGTIPVP
YRGNTYNIPICLWLLDTYPYNPPICFVKPTSSMTIKTGKHVDANGKIYLPYLHEWKHPQSDL
LGLIQVMIVVFGDEPPVFSRPISASYPPYQATGPPNTSYMPGMPGGISPYPSGYPPNPSGYP
GCPYPPGGPYPATTSSQYPSQPPVTTVGPSRDGTISEDTIRASLISAVSDKLRWRMKEEMDR
AQAELNALKRTEEDLKKGHQKLEEMVTRLDQEVAEVDKNIELLKKKDEELSSALEKMENQSE
NNDIDEVIIPTAPLYKQILNLYAEENAIEDTIFYLGEALRRGVIDLDVFLKHVRLLSRKQFQ
LRALMQKARKTAGLSDLY

>gi|11230780|ref|NP_068684.1|tumor susceptibility gene 101 protein [Mus musculus]

(SEQ ID NO: 46)
MAVSESQLKKMMSKYKYRDLTVRQTVNVIAMYKDLKPVLDSYVFNDGSSRELVNLTGTIPVR
YRGNIYNIPICLWLLDTYPYNPPICFVKPTSSMTIKTGKHVDANGKIYLPYLHDWKHPRSEL
LELIQIMIVIFGEEPPVFSRPTVSASYPPYTATGPPNTSYMPGMPSGISAYPSGYPPNPSGY
PGCPYPPAGPYPATTSSQYPSQPPVTTVGPSRDGTISEDTIRASLISAVSDKLRWRMKEEMD
GAQAELNALKRTEEDLKKGHQKLEEMVTRLDQEVAEVDKNIELLKKKDEELSSALEKMENQS
ENNDIDEVIIPTAPLYKQILNLYAEENAIEDTIFYLGEALRRGVIDLDVFLKHVRLLSRKQF
QLRALMQKARKTAGLSDLY

>gi|48374087|ref|NP_853659.2|tumor susceptibility gene 101 protein [Rattus norvegicus]

(SEQ ID NO: 47)
MAVSESQLKKMMSKYKYRDLTVRQTVNVIAMYKDLKPVLDSYVFNDGSSRELVNLTGTIPVR
YRGNIYNIPICLWLLDTYPYNPPICFVKPTSSMTIKTGKHVDANGKIYLPYLHDWKHPRSEL
LELIQIMIVIFGEEPPVFSRPTVSASYPPYTAAGPPNTSYLPSMPSGISAYPSGYPPNPSGY
PGCPYPPAGPYPATTSSQYPSQPPVTTAGPSRDGTISEDTIRASLISAVSDKLRWRMKEEMD
GAQAELNALKRTEEDLKKGHQKLEEMVTRLDQEVAEVDKNIELLKKKDEELSSALEKMENQS
ENNDIDEVIIPTAPLYKQILNLYAEENAIEDTIFYLGEALRRGVIDLDVFLKHVRLLSRKQF
QLRALMQKARKTAGLSDLY

The structure of UEV domains is known to those of skill in the art (See, e.g., Owen Pornillos et al., Structure and functional interactions of the Tsg101 UEV domain, EMBO J., 21(10): 2397-2406 (2002), the entire contents of which are incorporated herein by reference).

### Expression Constructs

Some aspects of this invention provide expression constructs for encoding a gene product or gene products that induce or facilitate the generation of ARMMs in cells harboring such a construct. In some embodiments, the expression constructs described herein encode a fusion proteins as described herein, such as ARRDC1 fusion proteins and TSG101 fusion proteins. In some embodiments, the expression constructs encode an ARRDC1 protein, or variant thereof, and/or a TSG101 protein, or variant thereof. In some embodiments, overexpression of either or both gene products in a cell increases the production of ARMMs in the cell, thus turning the cell into a microvesicle producing cell. In some embodiments, such an expression construct comprises at least one restriction or recombination site that allows in-frame cloning of a protein sequence to be fused, either at the C-terminus, or at the N-terminus of the encoded ARRDC1, or variant thereof. As another example, an expression construct comprises at least one restriction or recombination site that allows in-frame cloning of a protein sequence to be fused either at the C-terminus or at the N-terminus of one or more encoded WW domains.

In some embodiments, the expression construct comprises (a) a nucleotide sequence encoding an ARRDC1 protein, or variant thereof, operably linked to a heterologous promoter, and (b) a restriction site or a recombination site positioned adjacent to the ARRDC1-encoding nucleotide sequence allowing for the insertion of a nucleotide sequencing encoding a payload protein, or an RNA binding protein or RNA binding protein variant sequence, in frame with the ARRDC1-encoding nucleotide sequence. In some embodiments, the heterologous promoter may be a constitutive promoter, in some embodiments, the heterologous promoter may be an inducible promoter. Some aspects of this invention provide an expression construct comprising (a) a nucleotide sequence encoding a TSG101 protein, or variant thereof, operably linked to a heterologous promoter, and (b) a restriction site or a recombination site positioned adjacent to the TSG101-encoding nucleotide sequence allowing for the insertion of a nucleotide sequencing encoding a payload protein, or an RNA binding protein, DNA binding protein, or variant sequence thereof, in frame with the TSG101-encoding nucleotide sequence. In some embodiments, the heterologous promoter may be a constitutive promoter, in some embodiments, the heterologous promoter may be an inducible promoter.

Some aspects of this invention provide an expression construct comprising (a) a nucleotide sequence encoding a WW domain, or variant thereof, operably linked to a heterologous promoter, and (b) a restriction site or a recombination site positioned adjacent to the WW domain-encoding nucleotide sequence allowing for the insertion of a payload protein or RNA binding protein, or a protein variant sequence thereof in frame with the WW domain-encoding nucleotide sequence. In some embodiments, the heterologous promoter may be a constitutive promoter, in some embodiments, the heterologous promoter may be an inducible promoter. The expression constructs may encode a payload protein, or an RNA binding protein fused to at least one WW domain. In some embodiments, the expression constructs encode a payload protein or an RNA binding protein, or variant thereof, fused to at least one WW domain, or variant thereof. Any of the expression constructs, described herein, may encode any WW domain or variant thereof. In some embodiments, the heterologous promoter may be a constitutive promoter, in some embodiments, the heterologous promoter may be an inducible promoter.

The expression constructs, described herein, may comprise any nucleic acid sequence capable of encoding a WW domain or variant thereof. For example, a nucleic acid sequence encoding a WW domain or WW domain variant may be from the human ubiquitin ligase WWP1, WWP2, Nedd4-1, Nedd4-2, Smurf1, Smurf2, ITCH, NEDL1, or NEDL2. Exemplary nucleic acid sequences of WW domain containing proteins are listed below. It should be appreciated that any of the nucleic acids encoding WW domains or WW domain variants of the exemplary proteins may be used in the invention, described herein, and are not meant to be limiting.

Human WWP1 nucleic acid sequence (uniprot.org/uniprot/Q9H0M0).

(SEQ ID NO: 48)
GAATTCGCGGCCGCGTCGACCGCTTCTGTGGCCACGGCAGATGAAACAGAAAGGCTAAAG
AGGGCTGGAGTCAGGGGACTTCTCTTCCACCAGCTTCACGGTGATGATATGGCATCTGCC
AGCTCTAGCCGGGCAGGAGTGGCCCTGCCTTTTGAGAAGTCTCAGCTCACTTTGAAAGTG
GTGTCCGCAAAGCCCAAGGTGCATAATCGTCAACCTCGAATTAACTCCTACGTGGAGGTG
GCGGTGGATGGACTCCCCAGTGAGACCAAGAAGACTGGGAAGCGCATTGGGAGCTCTGAG
CTTCTCTGGAATGAGATCATCATTTTGAATGTCACGGCACAGAGTCATTTAGATTTAAAG
GTCTGGAGCTGCCATACCTTGAGAAATGAACTGCTAGGCACCGCATCTGTCAACCTCTCC
AACGTCTTGAAGAACAATGGGGGCAAAATGGAGAACATGCAGCTGACCCTGAACCTGCAG
ACGGAGAACAAAGGCAGCGTTGTCTCAGGCGGAAAACTGACAATTTTCCTGGACGGGCCA
ACTGTTGATCTGGGAAATGTGCCTAATGGCAGTGCCCTGACAGATGGATCACAGCTGCCT
TCGAGAGACTCCAGTGGAACAGCAGTAGCTCCAGAGAACCGGCACCAGCCCCCCAGCACA
AACTGCTTTGGTGGAAGATCCCGGACGCACAGACATTCGGGTGCTTCAGCCAGAACAACC
CCAGCAACCGGCGAGCAAAGCCCCGGTGCTCGGAGCCGGCACCGCCAGCCCGTCAAGAAC
TCAGGCCACAGTGGCTTGGCCAATGGCACAGTGAATGATGAACCCACAACAGCCACTGAT
CCCGAAGAACCTTCCGTTGTTGGTGTGACGTCCCCACCTGCTGCACCCTTGAGTGTGACC
CCGAATCCCAACACGACTTCTCTCCCTGCCCCAGCCACACCGGCTGAAGGAGAGGAACCC
AGCACTTCGGGTACACAGCAGCTCCCAGCGGCTGCCCAGGCCCCCGACGCTCTGCCTGCT
GGATGGGAACAGCGAGAGCTGCCCAACGGACGTGTCTATTATGTTGACCACAATACCAAG
ACCACCACCTGGGAGCGGCCCCTTCCTCCAGGCTGGGAAAAACGCACAGATCCCCGAGGC
AGGTTTTACTATGTGGATCACAATACTCGGACCACCACCTGGCAGCGTCCGACCGCGGAG
TACGTGCGCAACTATGAGCAGTGGCAGTCGCAGCGGAATCAGCTCCAGGGGGCCATGCAG
CACTTCAGCCAAAGATTCCTATACCAGTTTTGGAGTGCTTCGACTGACCATGATCCCCTG
GGCCCCCTCCCTCCTGGTTGGGAGAAAAGACAGGACAATGGACGGGTGTATTACGTGAAC
CATAACACTCGCACGACCCAGTGGGAGGATCCCCGGACCCAGGGGATGATCCAGGAACCA
GCTTTGCCCCCAGGATGGGAGATGAAATACACCAGCGAGGGGGTGCGATACTTTGTGGAC
CACAATACCCGCACCACCACCTTTAAGGATCCTCGCCCGGGGTTTGAGTCGGGGACGAAG
CAAGGTTCCCCTGGTGCTTATGACCGCAGTTTTCGGTGGAAGTATCACCAGTTCCGTTTC
CTCTGCCATTCAAATGCCCTACCTAGCCACGTGAAGATCAGCGTTTCCAGGCAGACGCTT
TTCGAAGATTCCTTCCAACAGATCATGAACATGAAACCCTATGACCTGCGCCGCCGGCTT
TACATCATCATGCGTGGCGAGGAGGGCCTGGACTATGGGGGCATCGCCAGAGAGTGGTTT
TTCCTCCTGTCTCACGAGGTGCTCAACCCTATGTATTGTTTATTTGAATATGCCGGAAAG
AACAATTACTGCCTGCAGATCAACCCCGCCTCCTCCATCAACCCGGACCACCTCACCTAC
TTTCGCTTTATAGGCAGATTCATCGCCATGGCGCTGTACCATGGAAAGTTCATCGACACG
GGCTTCACCCTCCCTTTCTACAAGCGGATGCTCAATAAGAGACCAACCCTGAAAGACCTG
GAGTCCATTGACCCTGAGTTCTACAACTCCATTGTCTGGATCAAAGAGAACAACCTGGAA
GAATGTGGCCTGGAGCTGTACTTCATCCAGGACATGGAGATACTGGGCAAGGTGACGACC
CACGAGCTGAAGGAGGGCGGCGAGAGCATCCGGGTCACGGAGGAGAACAAGGAAGAGTAC
ATCATGCTGCTGACTGACTGGCGTTTCACCCGAGGCGTGGAAGAGCAGACCAAAGCCTTC
CTGGATGGCTTCAACGAGGTGGCCCCGCTGGAGTGGCTGCGCTACTTTGACGAGAAAGAG
CTGGAGCTGATGCTGTGCGGCATGCAGGAGATAGACATGAGCGACTGGCAGAAGAGCACC
ATCTACCGGCACTACACCAAGAACAGCAAGCAGATCCAGTGGTTCTGGCAGGTGGTGAAG
GAGATGGACAACGAGAAGAGGATCCGGCTGCTGCAGTTTGTCACCGGTACCTGCCGCCTG
CCCGTCGGGGGATTTGCCGAACTCATCGGTAGCAACGGACCACAGAAGTTTTGCATTGAC
AAAGTTGGCAAGGAAACCTGGCTGCCCAGAAGCCACACCTGCTTCAACCGTCTGGATCTT
CCACCCTACAAGAGCTACGAACAGCTGAGAGAGAAGCTGCTGTATGCCATTGAGGAGACC
GAGGGCTTTGGACAGGAGTAACCGAGGCCGCCCCTCCCACGCCCCCCAGCGCACATGTAG
TCCTGAGTCCTCCCTGCCTGAGAGGCCACTGGCCCCGCAGCCCTTGGGAGGCCCCCGTGG
ATGTGGCCCTGTGTGGGACCACACTGTCATCTCGCTGCTGGCAGAAAAGCCTGATCCCAG
GAGGCCCTGCAGTTCCCCCGACCCGCGGATGGCAGTCTGGAATAAAGCCCCCTAGTTGCC
TTTGGCCCCACCTTTGCAAAGTTCCAGAGGGCTGACCCTCTCTGCAAAACTCTCCCCTGT
CCTCTAGACCCCACCCTGGGTGTATGTGAGTGTGCAAGGGAAGGTGTTGCATCCCCAGGG
GCTGCCGCAGAGGCCGGAGACCTCCTGGACTAGTTCGGCGAGGAGACTGGCCACTGGGGG
TGGCTGTTCGGGACTGAGAGCGCCAAGGGTCTTTGCCAGCAAAGGAGGTTCTGCCTGTAA
TTGAGCCTCTCTGATGATGGAGATGAAGTGAAGGTCTGAGGGACGGGCCCTGGGGCTAGG
CCATCTCTGCCTGCCTCCCTAGCAGGCGCCAGCGGTGGAGGCTGAGTCGCAGGACACATG
CCGGCCAGTTAATTCATTCTCAGCAAATGAAGGTTTGTCTAAGCTGCCTGGGTATCCACG
GGACAAAAACAGCAAACTCCCTCCAGACTTTGTCCATGTTATAAACTTGAAAGTTGGTTG
TTGTTTGTTAGGTTTGCCAGGTTTTTTTGTTTACGCCTGCTGTCACTTTCCTGTC
Human WWP2 nucleic acid sequence (uniprot.org/uniprot/O00308).
(SEQ ID NO: 49)
GAATTCGCGGCCGCGTCGACCGCTTCTGTGGCCACGGCAGATGAAACAGAAAGGCTAAAG
AGGGCTGGAGTCAGGGGACTTCTCTTCCACCAGCTTCACGGTGATGATATGGCATCTGCC
AGCTCTAGCCGGGCAGGAGTGGCCCTGCCTTTTGAGAAGTCTCAGCTCACTTTGAAAGTG
GTGTCCGCAAAGCCCAAGGTGCATAATCGTCAACCTCGAATTAACTCCTACGTGGAGGTG
GCGGTGGATGGACTCCCCAGTGAGACCAAGAAGACTGGGAAGCGCATTGGGAGCTCTGAG
CTTCTCTGGAATGAGATCATCATTTTGAATGTCACGGCACAGAGTCATTTAGATTTAAAG
GTCTGGAGCTGCCATACCTTGAGAAATGAACTGCTAGGCACCGCATCTGTCAACCTCTCC
AACGTCTTGAAGAACAATGGGGGCAAAATGGAGAACATGCAGCTGACCCTGAACCTGCAG
ACGGAGAACAAAGGCAGCGTTGTCTCAGGCGGAAAACTGACAATTTTCCTGGACGGGCCA
ACTGTTGATCTGGGAAATGTGCCTAATGGCAGTGCCCTGACAGATGGATCACAGCTGCCT
TCGAGAGACTCCAGTGGAACAGCAGTAGCTCCAGAGAACCGGCACCAGCCCCCCAGCACA
AACTGCTTTGGTGGAAGATCCCGGACGCACAGACATTCGGGTGCTTCAGCCAGAACAACC
CCAGCAACCGGCGAGCAAAGCCCCGGTGCTCGGAGCCGGCACCGCCAGCCCGTCAAGAAC
TCAGGCCACAGTGGCTTGGCCAATGGCACAGTGAATGATGAACCCACAACAGCCACTGAT
CCCGAAGAACCTTCCGTTGTTGGTGTGACGTCCCCACCTGCTGCACCCTTGAGTGTGACC
CCGAATCCCAACACGACTTCTCTCCCTGCCCCAGCCACACCGGCTGAAGGAGAGGAACCC
AGCACTTCGGGTACACAGCAGCTCCCAGCGGCTGCCCAGGCCCCCGACGCTCTGCCTGCT
GGATGGGAACAGCGAGAGCTGCCCAACGGACGTGTCTATTATGTTGACCACAATACCAAG
ACCACCACCTGGGAGCGGCCCCTTCCTCCAGGCTGGGAAAAACGCACAGATCCCCGAGGC
AGGTTTTACTATGTGGATCACAATACTCGGACCACCACCTGGCAGCGTCCGACCGCGGAG
TACGTGCGCAACTATGAGCAGTGGCAGTCGCAGCGGAATCAGCTCCAGGGGGCCATGCAG
CACTTCAGCCAAAGATTCCTATACCAGTTTTGGAGTGCTTCGACTGACCATGATCCCCTG
GGCCCCCTCCCTCCTGGTTGGGAGAAAAGACAGGACAATGGACGGGTGTATTACGTGAAC
CATAACACTCGCACGACCCAGTGGGAGGATCCCCGGACCCAGGGGATGATCCAGGAACCA
GCTTTGCCCCCAGGATGGGAGATGAAATACACCAGCGAGGGGGTGCGATACTTTGTGGAC
CACAATACCCGCACCACCACCTTTAAGGATCCTCGCCCGGGGTTTGAGTCGGGGACGAAG
CAAGGTTCCCCTGGTGCTTATGACCGCAGTTTTCGGTGGAAGTATCACCAGTTCCGTTTC
CTCTGCCATTCAAATGCCCTACCTAGCCACGTGAAGATCAGCGTTTCCAGGCAGACGCTT
TTCGAAGATTCCTTCCAACAGATCATGAACATGAAACCCTATGACCTGCGCCGCCGGCTT
TACATCATCATGCGTGGCGAGGAGGGCCTGGACTATGGGGGCATCGCCAGAGAGTGGTTT
TTCCTCCTGTCTCACGAGGTGCTCAACCCTATGTATTGTTTATTTGAATATGCCGGAAAG
AACAATTACTGCCTGCAGATCAACCCCGCCTCCTCCATCAACCCGGACCACCTCACCTAC
TTTCGCTTTATAGGCAGATTCATCGCCATGGCGCTGTACCATGGAAAGTTCATCGACACG
GGCTTCACCCTCCCTTTCTACAAGCGGATGCTCAATAAGAGACCAACCCTGAAAGACCTG
GAGTCCATTGACCCTGAGTTCTACAACTCCATTGTCTGGATCAAAGAGAACAACCTGGAA
GAATGTGGCCTGGAGCTGTACTTCATCCAGGACATGGAGATACTGGGCAAGGTGACGACC
CACGAGCTGAAGGAGGGCGGCGAGAGCATCCGGGTCACGGAGGAGAACAAGGAAGAGTAC
ATCATGCTGCTGACTGACTGGCGTTTCACCCGAGGCGTGGAAGAGCAGACCAAAGCCTTC
CTGGATGGCTTCAACGAGGTGGCCCCGCTGGAGTGGCTGCGCTACTTTGACGAGAAAGAG
CTGGAGCTGATGCTGTGCGGCATGCAGGAGATAGACATGAGCGACTGGCAGAAGAGCACC
ATCTACCGGCACTACACCAAGAACAGCAAGCAGATCCAGTGGTTCTGGCAGGTGGTGAAG
GAGATGGACAACGAGAAGAGGATCCGGCTGCTGCAGTTTGTCACCGGTACCTGCCGCCTG
CCCGTCGGGGGATTTGCCGAACTCATCGGTAGCAACGGACCACAGAAGTTTTGCATTGAC
AAAGTTGGCAAGGAAACCTGGCTGCCCAGAAGCCACACCTGCTTCAACCGTCTGGATCTT
CCACCCTACAAGAGCTACGAACAGCTGAGAGAGAAGCTGCTGTATGCCATTGAGGAGACC
GAGGGCTTTGGACAGGAGTAACCGAGGCCGCCCCTCCCACGCCCCCCAGCGCACATGTAG
TCCTGAGTCCTCCCTGCCTGAGAGGCCACTGGCCCCGCAGCCCTTGGGAGGCCCCCGTGG
ATGTGGCCCTGTGTGGGACCACACTGTCATCTCGCTGCTGGCAGAAAAGCCTGATCCCAG
GAGGCCCTGCAGTTCCCCCGACCCGCGGATGGCAGTCTGGAATAAAGCCCCCTAGTTGCC
TTTGGCCCCACCTTTGCAAAGTTCCAGAGGGCTGACCCTCTCTGCAAAACTCTCCCCTGT
CCTCTAGACCCCACCCTGGGTGTATGTGAGTGTGCAAGGGAAGGTGTTGCATCCCCAGGG
GCTGCCGCAGAGGCCGGAGACCTCCTGGACTAGTTCGGCGAGGAGACTGGCCACTGGGGG
TGGCTGTTCGGGACTGAGAGCGCCAAGGGTCTTTGCCAGCAAAGGAGGTTCTGCCTGTAA
TTGAGCCTCTCTGATGATGGAGATGAAGTGAAGGTCTGAGGGACGGGCCCTGGGGCTAGG
CCATCTCTGCCTGCCTCCCTAGCAGGCGCCAGCGGTGGAGGCTGAGTCGCAGGACACATG
CCGGCCAGTTAATTCATTCTCAGCAAATGAAGGTTTGTCTAAGCTGCCTGGGTATCCACG
GGACAAAAACAGCAAACTCCCTCCAGACTTTGTCCATGTTATAAACTTGAAAGTTGGTTG
TTGTTTGTTAGGTTTGCCAGGTTTTTTTGTTTACGCCTGCTGTCACTTTCCTGTC
Human Nedd4-1 nucleic acid sequence (uniprot.org/uniprot/P46934).
(SEQ ID NO: 50)
ACAGTTGCCTGCCCTGGGCGGGGGCGAGCGCGTCCGGTTTGCTGGAAGCGTTCGGAAATG
GCAACTTGCGCGGTGGAGGTGTTCGGGCTCCTGGAGGACGAGGAAAATTCACGAATTGTG
AGAGTAAGAGTTATAGCCGGAATAGGCCTTGCCAAGAAGGATATATTGGGAGCTAGTGAT
CCTTACGTGAGAGTGACGTTATATGACCCAATGAATGGAGTTCTTACAAGTGTGCAAACA
AAAACCATTAAAAAGAGTTTGAATCCAAAGTGGAATGAAGAAATATTATTCAGAGTTCAT
CCTCAGCAGCACCGGCTTCTTTTTGAAGTGTTTGACGAAAACCGATTGACAAGAGATGAT
TTCCTAGGTCAAGTGGATGTTCCACTTTATCCATTACCGACAGAAAATCCAAGATTGGAG
AGACCATATACATTTAAGGATTTTGTTCTTCATCCAAGAAGTCACAAATCAAGAGTTAAA
GGTTATCTGAGACTAAAAATGACTTATTTACCTAAAACCAGTGGCTCAGAAGATGATAAT
GCAGAACAGGCTGAGGAATTAGAGCCTGGCTGGGTTGTTTTGGACCAACCAGATGCTGCT
TGCCATTTGCAGCAACAACAAGAACCTTCTCCTCTACCTCCAGGGTGGGAAGAGAGGCAG
GATATCCTTGGAAGGACCTATTATGTAAACCATGAATCTAGAAGAACACAGTGGAAAAGA
CCAACCCCTCAGGACAACCTAACAGATGCTGAGAATGGCAACATTCAACTGCAAGCACAA
CGTGCATTTACCACCAGGCGGCAGATATCCGAGGAAACAGAAAGTGTTGACAACCAAGAG
TCTTCCGAGAACTGGGAAATTATAAGAGAAGATGAAGCCACCATGTATAGCAGCCAGGCC
TTCCCATCACCTCCACCGTCAAGTAACTTGGATGTTCCAACTCATCTTGCAGAAGAATTG
AATGCCAGACTCACCATTTTTGGAAATTCAGCCGTGAGCCAGCCAGCATCGAGCTCAAAT
CATTCCAGCAGAAGAGGCAGCTTACAAGCCTATACTTTTGAGGAACAACCTACACTTCCT
GTGCTTTTGCCTACTTCATCTGGATTACCACCAGGTTGGGAAGAAAAACAAGATGAAAGA
GGAAGATCATATTATGTAGATCACAATTCCAGAACGACTACTTGGACAAAGCCCACTGTA
CAGGCCACAGTGGAGACCAGTCAGCTGACCTCAAGCCAGAGTTCTGCAGGCCCTCAATCA
CAAGCCTCCACCAGTGATTCAGGCCAGCAGGTGACCCAGCCATCTGAAATTGAGCAAGGA
TTCCTTCCTAAAGGCTGGGAAGTCCGGCATGCACCAAATGGGAGGCCTTTCTTTATTGAC
CACAACACTAAAACCACCACCTGGGAAGATCCAAGATTGAAAATTCCAGCCCATCTGAGA
GGAAAGACATCACTTGATACTTCCAATGATCTAGGGCCTTTACCTCCAGGATGGGAAGAG
AGAACTCACACAGATGGAAGAATCTTCTACATAAATCACAATATAAAAAGAACACAATGG
GAAGATCCTCGGTTGGAGAATGTAGCAATAACTGGACCAGCAGTGCCCTACTCCAGGGAT
TACAAAAGAAAGTATGAGTTCTTCCGAAGAAAGTTGAAGAAGCAGAATGACATTCCAAAC
AAATTTGAAATGAAACTTCGCCGAGCAACTGTTCTTGAAGACTCTTACCGGAGAATTATG
GGTGTCAAGAGAGCAGACTTCCTGAAGGCTCGACTGTGGATTGAGTTTGATGGTGAAAAG
GGATTGGATTATGGAGGAGTTGCCAGAGAATGGTTCTTCCTGATCTCAAAGGAAATGTTT
AACCCTTATTATGGGTTGTTTGAATATTCTGCTACGGACAATTATACCCTACAGATAAAT
CCAAACTCTGGATTGTGTAACGAAGATCACCTCTCTTACTTCAAGTTTATTGGTCGGGTA
GCTGGAATGGCAGTTTATCATGGCAAACTGTTGGATGGTTTTTTCATCCGCCCATTTTAC
AAGATGATGCTTCACAAACCAATAACCCTTCATGATATGGAATCTGTGGATAGTGAATAT
TACAATTCCCTAAGATGGATTCTTGAAAATGACCCAACAGAATTGGACCTCAGGTTTATC
ATAGATGAAGAACTTTTTGGACAGACACATCAACATGAGCTGAAAAATGGTGGATCAGAA
ATAGTTGTCACCAATAAGAACAAAAAGGAATATATTTATCTTGTAATACAATGGCGATTT
GTAAACCGAATCCAGAAGCAAATGGCTGCTTTTAAAGAGGGATTCTTTGAACTAATACCA
CAGGATCTCATCAAAATTTTTGATGAAAATGAACTAGAGCTTCTTATGTGTGGACCGGGA
GATGTTGATGTGAATGACTGGAGGGAACATACAAAGTATAAAAATGGCTACAGTGCAAAT
CATCAGGTTATACAGTGGTTTTGGAAGGCTGTTTTAATGATGGATTCAGAAAAAAGAATA
AGATTACTTCAGTTTGTCACTGGCACATCTCGGGTGCCTATGAATGGATTTGCTGAACTA
TACGGTTCAAATGGACCACAGTCATTTACAGTTGAACAGTGGGGTACTCCTGAAAAGCTG
CCAAGAGCTCATACCTGITTTAATCGCCTGGACTTGCCACCTTATGAATCATTTGAAGAA
TTATGGGATAAACTTCAGATGGCAATTGAAAACACCCAGGGCTTTGATGGAGTTGATTAG
ATTACAAATAACAATCTGTAGTGTTTTTACTGCCATAGTTTTATAACCAAAATCTTGACT
TAAAATTTTCCGGGGAACTACTAAAATGTGGCCACTGAGTCTTCCCAGATCTTGAAGAAA
ATCATATAAAAAGCATTTGAAGAAATAGTACGAC

Human Nedd4-2 nucleic acid sequence (>gi|345478679|ref|NM_015277.51 Homo sapiens neural precursor cell expressed, developmentally down-regulated 4-like, E3 ubiquitin protein ligase (NEDD4L), transcript variant d, mRNA).

(SEQ ID NO: 51)
ATGGCGACCGGGCTCGGGGAGCCGGTCTATGGACTTTCCGAAGACGAGGGAGAGTCCCGTAT
TCTCAGAGTAAAAGTTGTTTCTGGAATTGATCTCGCCAAAAAGGACATCTTTGGAGCCAGTG
ATCCGTATGTGAAACTTTCATTGTACGTAGCGGATGAGAATAGAGAACTTGCTTTGGTCCAG
ACAAAAACAATTAAAAAGACACTGAACCCAAAATGGAATGAAGAATTTTATTTCAGGGTAAA
CCCATCTAATCACAGACTCCTATTTGAAGTATTTGACGAAAATAGACTGACACGAGACGACT
TCCTGGGCCAGGTGGACGTGCCCCTTAGTCACCTTCCGACAGAAGATCCAACCATGGAGCGA
CCCTATACATTTAAGGACTTTCTCCTCAGACCAAGAAGTCATAAGTCTCGAGTTAAGGGATT
TTTGCGATTGAAAATGGCCTATATGCCAAAAAATGGAGGTCAAGATGAAGAAAACAGTGACC
AGAGGGATGACATGGAGCATGGATGGGAAGTTGTTGACTCAAATGACTCGGCTTCTCAGCAC
CAAGAGGAACTTCCTCCTCCTCCTCTGCCTCCCGGGTGGGAAGAAAAAGTGGACAATTTAGG
CCGAACTTACTATGTCAACCACAACAACCGGACCACTCAGTGGCACAGACCAAGCCTGATGG
ACGTGTCCTCGGAGTCGGACAATAACATCAGACAGATCAACCAGGAGGCAGCACACCGGCGC
TTCCGCTCCCGCAGGCACATCAGCGAAGACTTGGAGCCCGAGCCCTCGGAGGGCGGGGATGT
CCCCGAGCCTTGGGAGACCATTTCAGAGGAAGTGAATATCGCTGGAGACTCTCTCGGTCTGG
CTCTGCCCCCACCACCGGCCTCCCCAGGATCTCGGACCAGCCCTCAGGAGCTGTCAGAGGAA
CTAAGCAGAAGGCTTCAGATCACTCCAGACTCCAATGGGGAACAGTTCAGCTCTTTGATTCA
AAGAGAACCCTCCTCAAGGTTGAGGTCATGCAGTGTCACCGACGCAGTTGCAGAACAGGGCC
ATCTACCACCGCCATCAGTGGCCTATGTACATACCACGCCGGGTCTGCCTTCAGGCTGGGAA
GAAAGAAAAGATGCTAAGGGGCGCACATACTATGTCAATCATAACAATCGAACCACAACTTG
GACTCGACCTATCATGCAGCTTGCAGAAGATGGTGCGTCCGGATCAGCCACAAACAGTAACA
ACCATCTAATCGAGCCTCAGATCCGCCGGCCTCGTAGCCTCAGCTCGCCAACAGTAACTTTA
TCTGCCCCGCTGGAGGGTGCCAAGGACTCACCCGTACGTCGGGCTGTGAAAGACACCCTTTC
CAACCCACAGTCCCCACAGCCATCACCTTACAACTCCCCCAAACCACAACACAAAGTCACAC
AGAGCTTCTTGCCACCCGGCTGGGAAATGAGGATAGCGCCAAACGGCCGGCCCTTCTTCATT
GATCATAACACAAAGACTACAACCTGGGAAGATCCACGTTTGAAATTTCCAGTACATATGCG
GTCAAAGACATCTTTAAACCCCAATGACCTTGGCCCCCTTCCTCCTGGCTGGGAAGAAAGAA
TTCACTTGGATGGCCGAACGTTTTATATTGATCATAATAGCAAAATTACTCAGTGGGAAGAC
CCAAGACTGCAGAACCCAGCTATTACTGGTCCGGCTGTCCCTTACTCCAGAGAATTTAAGCA
GAAATATGACTACTTCAGGAAGAAATTAAAGAAACCTGCTGATATCCCCAATAGGTTTGAAA
TGAAACTTCACAGAAATAACATATTTGAAGAGTCCTATCGGAGAATTATGTCCGTGAAAAGA
CCAGATGTCCTAAAAGCTAGACTGTGGATTGAGTTTGAATCAGAGAAAGGTCTTGACTATGG
GGGTGTGGCCAGAGAATGGTTCTTCTTACTGTCCAAAGAGATGTTCAACCCCTACTACGGCC
TCTTTGAGTACTCTGCCACGGACAACTACACCCTTCAGATCAACCCTAATTCAGGCCTCTGT
AATGAGGATCATTTGTCCTACTTCACTTTTATTGGAAGAGTTGCTGGTCTGGCCGTATTTCA
TGGGAAGCTCTTAGATGGTTTCTTCATTAGACCATTTTACAAGATGATGTTGGGAAAGCAGA
TAACCCTGAATGACATGGAATCTGTGGATAGTGAATATTACAACTCTTTGAAATGGATCCTG
GAGAATGACCCTACTGAGCTGGACCTCATGTTCTGCATAGACGAAGAAAACTTTGGACAGAC
ATATCAAGTGGATTTGAAGCCCAATGGGTCAGAAATAATGGTCACAAATGAAAACAAAAGGG
AATATATCGACTTAGTCATCCAGTGGAGATTTGTGAACAGGGTCCAGAAGCAGATGAACGCC
TTCTTGGAGGGATTCACAGAACTACTTCCTATTGATTTGATTAAAATTTTTGATGAAAATGA
GCTGGAGTTGCTCATGTGCGGCCTCGGTGATGTGGATGTGAATGACTGGAGACAGCATTCTA
TTTACAAGAACGGCTACTGCCCAAACCACCCCGTCATTCAGTGGTTCTGGAAGGCTGTGCTA
CTCATGGACGCCGAAAAGCGTATCCGGTTACTGCAGTTTGTCACAGGGACATCGCGAGTACC
TATGAATGGATTTGCCGAACTTTATGGTTCCAATGGTCCTCAGCTGTTTACAATAGAGCAAT
GGGGCAGTCCTGAGAAACTGCCCAGAGCTCACACATGCTTTAATCGCCTTGACTTACCTCCA
TATGAAACCTTTGAAGATTTACGAGAGAAACTTCTCATGGCCGTGGAAAATGCTCAAGGATT
TGAAGGGGTGGATTAA
Human Smurf1 nucleic acid sequence (uniprot.org/uniprot/Q9HCE7).
(SEQ ID NO: 52)
ATGTCGAACCCCGGGACACGCAGGAACGGCTCCAGCATCAAGATCCGTCTGACAGTGTTA
TGTGCCAAGAACCTTGCAAAGAAAGACTTCTTCAGGCTCCCTGACCCTTTTGCAAAGATT
GTCGTGGATGGGTCTGGGCAGTGCCACTCAACCGACACTGTGAAAAACACATTGGACCCA
AAGTGGAACCAGCACTATGATCTATATGTTGGGAAAACGGATTCGATAACCATTAGCGTG
TGGAACCATAAGAAAATTCACAAGAAACAGGGAGCTGGCTTCCTGGGCTGTGTGCGGCTG
CTCTCCAATGCCATCAGCAGATTAAAAGATACCGGATACCAGCGTTTGGATCTATGCAAA
CTAAACCCCTCAGATACTGATGCAGTTCGTGGCCAGATAGTGGTCAGTTTACAGACACGA
GACAGAATAGGAACCGGCGGCTCGGTGGTGGACTGCAGAGGACTGTTAGAAAATGAAGGA
ACGGTGTATGAAGACTCCGGGCCTGGGAGGCCGCTCAGCTGCTTCATGGAGGAACCAGCC
CCTTACACAGATAGCACCGGTGCTGCTGCTGGAGGAGGGAATTGCAGGTTCGTGGAGTCC
CCAAGTCAAGATCAAAGACTTCAGGCACAGCGGCTTCGAAACCCTGATGTGCGAGGTTCA
CTACAGACGCCCCAGAACCGACCACACGGCCACCAGTCCCCGGAACTGCCCGAAGGCTAC
GAACAAAGAACAACAGTCCAGGGCCAAGTTTACTTTTTGCATACACAGACTGGAGTTAGC
ACGTGGCACGACCCCAGGATACCAAGTCCCTCGGGGACCATTCCTGGGGGAGATGCAGCT
TTTCTATACGAATTCCTTCTACAAGGCCATACATCTGAGCCCAGAGACCTTAACAGTGTG
AACTGTGATGAACTTGGACCACTGCCGCCAGGCTGGGAAGTCAGAAGTACAGTTTCTGGG
AGGATATATTTTGTAGATCATAATAACCGAACAACCCAGTTTACAGACCCAAGGTTACAC
CACATCATGAATCACCAGTGCCAACTCAAGGAGCCCAGCCAGCCGCTGCCACTGCCCAGT
GAGGGCTCTCTGGAGGACGAGGAGCTTCCTGCCCAGAGATACGAAAGAGATCTAGTCCAG
AAGCTGAAAGTCCTCAGACACGAACTGTCGCTTCAGCAGCCCCAAGCTGGTCATTGCCGC
ATCGAAGTGTCCAGAGAAGAAATCTTTGAGGAGTCTTACCGCCAGATAATGAAGATGCGA
CCGAAAGACTTGAAAAAACGGCTGATGGTGAAATTCCGTGGGGAAGAAGGTTTGGATTAC
GGTGGTGTGGCCAGGGAGTGGCTTTACTTGCTGTGCCATGAAATGCTGAATCCTTATTAC
GGGCTCTTCCAGTATTCTACGGACAATATTTACATGTTGCAAATAAATCCGGATTCTTCA
ATCAACCCCGACCACTTGTCTTATTTCCACTTTGTGGGGCGGATCATGGGGCTGGCTGTG
TTCCATGGACACTACATCAACGGGGGCTTCACAGTGCCCTTCTACAAGCAGCTGCTGGGG
AAGCCCATCCAGCTCTCAGATCTGGAATCTGTGGACCCAGAGCTGCATAAGAGCTTGGTG
TGGATCCTAGAGAACGACATCACGCCTGTACTGGACCACACCTTCTGCGTGGAACACAAC
GCCTTCGGGCGGATCCTGCAGCATGAACTGAAACCCAATGGCAGAAATGTGCCAGTCACA
GAGGAGAATAAGAAAGAATACGTCCGGTTGTATGTAAACTGGAGGTTTATGAGAGGAATC
GAAGCCCAGTTCTTAGCTCTGCAGAAGGGGTTCAATGAGCTCATCCCTCAACATCTGCTG
AAGCCTTTTGACCAGAAGGAACTGGAGCTGATCATAGGCGGCCTGGATAAAATAGACTTG
AACGACTGGAAGTCGAACACGCGGCTGAAGCACTGTGTGGCCGACAGCAACATCGTGCGG
TGGTTCTGGCAAGCGGTGGAGACGTTCGATGAAGAAAGGAGGGCCAGGCTCCTGCAGTTT
GTGACTGGGTCCACGCGAGTCCCGCTCCAAGGCTTCAAGGCTTTGCAAGGTTCTACAGGC
GCGGCAGGGCCCCGGCTGTTCACCATCCACCTGATAGACGCGAACACAGACAACCTTCCG
AAGGCCCATACCTGCTTTAACCGGATCGACATTCCACCATATGAGTCCTATGAGAAGCTC
TACGAGAAGCTGCTGACAGCCGTGGAGGAGACCTGCGGGTTTGCTGTGGAGTGA
Human Smurf2 nucleic acid sequence (uniprot.org/uniprot/Q9HAU4).
(SEQ ID NO: 53)
ATGTCTAACCCCGGACGCCGGAGGAACGGGCCCGTCAAGCTGCGCCTGACAGTACTCTGT
GCAAAAAACCTGGTGAAAAAGGATTTTTTCCGACTTCCTGATCCATTTGCTAAGGTGGTG
GTTGATGGATCTGGGCAATGCCATTCTACAGATACTGTGAAGAATACGCTTGATCCAAAG
TGGAATCAGCATTATGACCTGTATATTGGAAAGTCTGATTCAGTTACGATCAGTGTATGG
AATCACAAGAAGATCCATAAGAAACAAGGTGCTGGATTTCTCGGTTGTGTTCGTCTTCTT
TCCAATGCCATCAACCGCCTCAAAGACACTGGTTATCAGAGGTTGGATTTATGCAAACTC
GGGCCAAATGACAATGATACAGTTAGAGGACAGATAGTAGTAAGTCTTCAGTCCAGAGAC
CGAATAGGCACAGGAGGACAAGTTGTGGACTGCAGTCGTTTATTTGATAACGATTTACCA
GACGGCTGGGAAGAAAGGAGAACCGCCTCTGGAAGAATCCAGTATCTAAACCATATAACA
AGAACTACGCAATGGGAGCGCCCAACACGACCGGCATCCGAATATTCTAGCCCTGGCAGA
CCTCTTAGCTGCTTTGTTGATGAGAACACTCCAATTAGTGGAACAAATGGTGCAACATGT
GGACAGTCTTCAGATCCCAGGCTGGCAGAGAGGAGAGTCAGGTCACAACGACATAGAAAT
TACATGAGCAGAACACATTTACATACTCCTCCAGACCTACCAGAAGGCTATGAACAGAGG
ACAACGCAACAAGGCCAGGTGTATTTCTTACATACACAGACTGGTGTGAGCACATGGCAT
GATCCAAGAGTGCCCAGGGATCTTAGCAACATCAATTGTGAAGAGCTTGGTCCATTGCCT
CCTGGATGGGAGATCCGTAATACGGCAACAGGCAGAGTTTATTTCGTTGACCATAACAAC
AGAACAACACAATTTACAGATCCTCGGCTGTCTGCTAACTTGCATTTAGTTTTAAATCGG
CAGAACCAATTGAAAGACCAACAGCAACAGCAAGTGGTATCGTTATGTCCTGATGACACA
GAATGCCTGACAGTCCCAAGGTACAAGCGAGACCTGGTTCAGAAACTAAAAATTTTGCGG
CAAGAACTTTCCCAACAACAGCCTCAGGCAGGTCATTGCCGCATTGAGGTTTCCAGGGAA
GAGATTTTTGAGGAATCATATCGACAGGTCATGAAAATGAGACCAAAAGATCTCTGGAAG
CGATTAATGATAAAATTTCGTGGAGAAGAAGGCCTTGACTATGGAGGCGTTGCCAGGGAA
TGGTTGTATCTCTTGTCACATGAAATGTTGAATCCATACTATGGCCTCTTCCAGTATTCA
AGAGATGATATTTATACATTGCAGATCAATCCTGATTCTGCAGTTAATCCGGAACATTTA
TCCTATTTCCACTTTGTTGGACGAATAATGGGAATGGCTGTGTTTCATGGACATTATATT
GATGGTGGTTTCACATTGCCTTTTTATAAGCAATTGCTTGGGAAGTCAATTACCTTGGAT
GACATGGAGTTAGTAGATCCGGATCTTCACAACAGTTTAGTGTGGATACTTGAGAATGAT
ATTACAGGTGTTTTGGACCATACCTTCTGTGTTGAACATAATGCATATGGTGAAATTATT
CAGCATGAACTTAAACCAAATGGCAAAAGTATCCCTGTTAATGAAGAAAATAAAAAAGAA
TATGTCAGGCTCTATGTGAACTGGAGATTTTTACGAGGCATTGAGGCTCAATTCTTGGCT
CTGCAGAAAGGATTTAATGAAGTAATTCCACAACATCTGCTGAAGACATTTGATGAGAAG
GAGTTAGAGCTCATTATTTGTGGACTTGGAAAGATAGATGTTAATGACTGGAAGGTAAAC
ACCCGGTTAAAACACTGTACACCAGACAGCAACATTGTCAAATGGTTCTGGAAAGCTGTG
GAGTTTTTTGATGAAGAGCGACGAGCAAGATTGCTTCAGTTTGTGACAGGATCCTCTCGA
GTGCCTCTGCAGGGCTTCAAAGCATTGCAAGGTGCTGCAGGCCCGAGACTCTTTACCATA
CACCAGATTGATGCCTGCACTAACAACCTGCCGAAAGCCCACACTTGCTTCAATCGAATA
GACATTCCACCCTATGAAAGCTATGAAAAGCTATATGAAAAGCTGCTAACAGCCATTGAA
GAAACATGTGGATTTGCTGTGGAATGA
Human ITCH nucleic acid sequence (uniprot.org/uniprot/Q96J02).
(SEQ ID NO: 54)
GGAGTCGCCGCCGCCCCGAGTTCCGGTACCATGCATTTCACGGTGGCCTTGTGGAGACAA
CGCCTTAACCCAAGGAAGTGACTCAAACTGTGAGAACTCCAGGTTTTCCAACCTATTGGT
GGTATGTCTGACAGTGGATCACAACTTGGTTCAATGGGTAGCCTCACCATGAAATCACAG
CTTCAGATCACTGTCATCTCAGCAAAACTTAAGGAAAATAAGAAGAATTGGTTTGGACCA
AGTCCTTACGTAGAGGTCACAGTAGATGGACAGTCAAAGAAGACAGAAAAATGCAACAAC
ACAAACAGTCCCAAGTGGAAGCAACCCCTTACAGTTATCGTTACCCCTGTGAGTAAATTA
CATTTTCGTGTGTGGAGTCACCAGACACTGAAATCTGATGTTTTGTTGGGAACTGCTGCA
TTAGATATTTATGAAACATTAAAGTCAAACAATATGAAACTTGAAGAAGTAGTTGTGACT
TTGCAGCTTGGAGGTGACAAAGAGCCAACAGAGACAATAGGAGACTTGTCAATTTGTCTT
GATGGGCTACAGTTAGAGTCTGAAGTTGTTACCAATGGTGAAACTACATGTTCAGAAAGT
GCTTCTCAGAATGATGATGGCTCCAGATCCAAGGATGAAACAAGAGTGAGCACAAATGGA
TCAGATGACCCTGAAGATGCAGGAGCTGGTGAAAATAGGAGAGTCAGTGGGAATAATTCT
CCATCACTCTCAAATGGTGGTTTTAAACCTTCTAGACCTCCAAGACCTTCACGACCACCA
CCACCCACCCCACGTAGACCAGCATCTGTCAATGGTTCACCATCTGCCACTTCTGAAAGT
GATGGGTCTAGTACAGGCTCTCTGCCGCCGACAAATACAAATACAAATACATCTGAAGGA
GCAACATCTGGATTAATAATTCCTCTTACTATATCTGGAGGCTCAGGCCCTAGGCCATTA
AATCCTGTAACTCAAGCTCCCTTGCCACCTGGTTGGGAGCAGAGAGTGGACCAGCACGGG
CGAGTTTACTATGTAGATCATGTTGAGAAAAGAACAACATGGGATAGACCAGAACCTCTA
CCTCCTGGCTGGGAACGGCGGGTTGACAACATGGGACGTATTTATTATGTTGACCATTTC
ACAAGAACAACAACGTGGCAGAGGCCAACACTGGAATCCGTCCGGAACTATGAACAATGG
CAGCTACAGCGTAGTCAGCTTCAAGGAGCAATGCAGCAGTTTAACCAGAGATTCATTTAT
GGGAATCAAGATTTATTTGCTACATCACAAAGTAAAGAATTTGATCCTCTTGGTCCATTG
CCACCTGGATGGGAGAAGAGAACAGACAGCAATGGCAGAGTATATTTCGTCAACCACAAC
ACACGAATTACACAATGGGAAGACCCCAGAAGTCAAGGTCAATTAAATGAAAAGCCCTTA
CCTGAAGGTTGGGAAATGAGATTCACAGTGGATGGAATTCCATATTTTGTGGACCACAAT
AGAAGAACTACCACCTATATAGATCCCCGCACAGGAAAATCTGCCCTAGACAATGGACCT
CAGATAGCCTATGTTCGGGACTTCAAAGCAAAGGTTCAGTATTTCCGGTTCTGGTGTCAG
CAACTGGCCATGCCACAGCACATAAAGATTACAGTGACAAGAAAAACATTGTTTGAGGAT
TCCTTTCAACAGATAATGAGCTTCAGTCCCCAAGATCTGCGAAGACGTTTGTGGGTGATT
TTTCCAGGAGAAGAAGGTTTAGATTATGGAGGTGTAGCAAGAGAATGGTTCTTTCTTTTG
TCACATGAAGTGTTGAACCCAATGTATTGCCTGTTTGAATATGCAGGGAAGGATAACTAC
TGCTTGCAGATAAACCCCGCTTCTTACATCAATCCAGATCACCTGAAATATTTTCGTTTT
ATTGGCAGATTTATTGCCATGGCTCTGTTCCATGGGAAATTCATAGACACGGGTTTTTCT
TTACCATTCTATAAGCGTATCTTGAACAAACCAGTTGGACTCAAGGATTTAGAATCTATT
GATCCAGAATTTTACAATTCTCTCATCTGGGTTAAGGAAAACAATATTGAGGAATGTGAT
TTGGAAATGTACTTCTCCGTTGACAAAGAAATTCTAGGTGAAATTAAGAGTCATGATCTG
AAACCTAATGGTGGCAATATTCTTGTAACAGAAGAAAATAAAGAGGAATACATCAGAATG
GTAGCTGAGTGGAGGTTGTCTCGAGGTGTTGAAGAACAGACACAAGCTTTCTTTGAAGGC
TTTAATGAAATTCTTCCCCAGCAATATTTGCAATACTTTGATGCAAAGGAATTAGAGGTC
CTTTTATGTGGAATGCAAGAGATTGATTTGAATGACTGGCAAAGACATGCCATCTACCGT
CATTATGCAAGGACCAGCAAACAAATCATGTGGTTTTGGCAGTTTGTTAAAGAAATTGAT
AATGAGAAGAGAATGAGACTTCTGCAGTTTGTTACTGGAACCTGCCGATTGCCAGTAGGA
GGATTTGCTGATCTCATGGGGAGCAATGGACCACAGAAATTCTGCATTGAAAAAGTTGGG
AAAGAAAATTGGCTACCCAGAAGTCATACCTGTTTTAATCGCCTGGACCTGCCACCATAC
AAGAGCTATGAGCAACTGAAGGAAAAGCTGTTGTTTGCCATAGAAGAAACAGAAGGATTT
GGACAAGAGTAACTTCTGAGAACTTGCACCATGAATGGGCAAGAACTTATTTGCAATGTT
TGTCCTTCTCTGCCTGTTGCACATCTTGTAAAATTGGACAATGGCTCTTTAGAGAGTTAT
CTGAGTGTAAGTAAATTAATGTTCTCATTTAAAAAAAAAAAAAAAAAAA
Human NEDL1 nucleic acid sequence (uniprot.org/uniprot/Q76N89).
(SEQ ID NO: 55)
GCGCATCAGGCGCTGTTGTTGGAGCCGGAACACCGTGCGACTCTGACCGAACCGGCCCCC
TCCTCGCGCACACACTCGCCGAGCCGCGCGCGCCCCTCCGCCGTGACAGTGGCCGTGGCC
TCCGCTCTCTCGGGGCACCCGGCAGCCAGAGCGCAGCGAGAGCGGGCGGTCGCCAGGGTC
CCCTCCCCAGCCAGTCCCAGGCGCCCGGTGCACTATGCGGGGCACGTGCGCCCCCCAGCT
CTAATCTGCGCGCTGACAGGAGCATGATCTGTGCCCAGGCCAGGGCTGCCAAGGAATTGA
TGCGCGTACACGTGGTGGGTCATTATGCTGCTACACCTGTGTAGTGTGAAGAATCTGTAC
CAGAACAGGTTTTTAGGCCTGGCCGCCATGGCGTCTCCTTCTAGAAACTCCCAGAGCCGA
CGCCGGTGCAAGGAGCCGCTCCGATACAGCTACAACCCCGACCAGTTCCACAACATGGAC
CTCAGGGGCGGCCCCCACGATGGCGTCACCATTCCCCGCTCCACCAGCGACACTGACCTG
GTCACCTCGGACAGCCGCTCCACGCTCATGGTCAGCAGCTCCTACTATTCCATCGGGCAC
TCTCAGGACCTGGTCATCCACTGGGACATAAAGGAGGAAGTGGACGCTGGGGACTGGATT
GGCATGTACCTCATTGATGAGGTCTTGTCCGAAAACTTTCTGGACTATAAAAACCGTGGA
GTCAATGGTTCTCATCGGGGCCAGATCATCTGGAAGATCGATGCCAGCTCGTACTTTGTG
GAACCTGAAACTAAGATCTGCTTCAAATACTACCATGGAGTGAGTGGGGCCCTGCGAGCA
ACCACCCCCAGTGTCACGGTCAAAAACTCGGCAGCTCCTATTTTTAAAAGCATTGGTGCT
GATGAGACCGTCCAAGGACAAGGAAGTCGGAGGCTGATCAGCTTCTCTCTCTCAGATTTC
CAAGCCATGGGGTTGAAGAAAGGGATGTTTTTCAACCCAGACCCTTATCTGAAGATTTCC
ATTCAGCCTGGGAAACACAGCATCTTCCCCGCCCTCCCTCACCATGGACAGGAGAGGAGA
TCCAAGATCATAGGCAACACCGTGAACCCCATCTGGCAGGCCGAGCAATTCAGTTTTGTG
TCCTTGCCCACTGACGTGCTGGAAATTGAGGTGAAGGACAAGTTTGCCAAGAGCCGCCCC
ATCATCAAGCGCTTCTTGGGAAAGCTGTCGATGCCCGTTCAAAGACTCCTGGAGAGACAC
GCCATAGGGGATAGGGTGGTCAGCTACACACTTGGCCGCAGGCTTCCAACAGATCATGTG
AGTGGACAGCTGCAATTCCGATTTGAGATCACTTCCTCCATCCACCCAGATGATGAGGAG
ATTTCCCTGAGTACCGAGCCTGAGTCAGCCCAAATTCAGGACAGCCCCATGAACAACCTG
ATGGAAAGCGGCAGTGGGGAACCTCGGTCTGAGGCACCAGAGTCCTCTGAGAGCTGGAAG
CCAGAGCAGCTGGGTGAGGGCAGTGTCCCCGATGGTCCAGGGAACCAAAGCATAGAGCTT
TCCAGACCAGCTGAGGAAGCAGCAGTCATCACGGAGGCAGGAGACCAGGGCATGGTCTCT
GTGGGACCTGAAGGGGCTGGGGAGCTCCTGGCCCAGGTGCAAAAGGACATCCAGCCTGCC
CCCAGTGCAGAAGAGCTGGCCGAGCAGCTGGACCTGGGTGAGGAGGCATCAGCACTGCTG
CTGGAAGACGGTGAAGCCCCAGCCAGCACCAAGGAGGAGCCCTTGGAGGAGGAAGCAACG
ACCCAGAGCCGGGCTGGAAGGGAAGAAGAGGAGAAGGAGCAGGAGGAGGAGGGAGATGTG
TCTACCCTGGAGCAGGGAGAGGGCAGGCTGCAGCTGCGGGCCTCGGTGAAGAGAAAAAGC
AGGCCCTGCTCCTTGCCTGTGTCCGAGCTGGAGACGGTGATCGCGTCAGCCTGCGGGGAC
CCCGAGACCCCGCGGACACACTACATCCGCATCCACACCCTGCTGCACAGCATGCCCTCC
GCCCAGGGCGGCAGCGCGGCAGAGGAGGAGGACGGCGCGGAGGAGGAGTCCACCCTCAAG
GACTCCTCGGAGAAGGATGGGCTCAGCGAGGTGGACACGGTGGCCGCTGACCCGTCTGCC
CTGGAAGAGGACAGAGAAGAGCCCGAGGGGGCTACTCCAGGCACGGCGCACCCTGGCCAC
TCCGGGGGCCACTTCCCCAGCCTGGCCAATGGCGCGGCCCAGGATGGCGACACGCACCCC
AGCACCGGGAGCGAGAGCGACTCCAGCCCCAGGCAAGGCGGGGACCACAGTTGCGAGGGC
TGTGACGCGTCCTGCTGCAGCCCCTCGTGCTACAGCTCCTCGTGCTACAGCACGTCCTGC
TACAGCAGCTCGTGCTACAGCGCCTCGTGCTACAGCCCCTCCTGCTACAACGGCAACAGG
TTCGCCAGCCACACGCGCTTCTCCTCCGTGGACAGCGCCAAGATCTCCGAGAGCACGGTC
TTCTCCTCGCAAGACGACGAGGAGGAGGAGAACAGCGCGTTCGAGTCGGTACCCGACTCC
ATGCAGAGCCCTGAGCTGGACCCGGAGTCCACGAACGGCGCTGGGCCGTGGCAAGACGAG
CTGGCCGCCCCTAGCGGGCACGTGGAAAGAAGCCCGGAAGGTCTGGAATCCCCCGTGGCA
GGTCCAAGCAATCGGAGAGAAGACTGGGAAGCTCGAATTGACAGCCACGGGCGGGTCTTT
TATGTGGACCACGTGAACCGCACAACCACCTGGCAGCGTCCGACGGCAGCAGCCACCCCG
GATGGCATGCGGAGATCGGGGTCCATCCAGCAGATGGAGCAACTCAACAGGCGGTATCAA
AACATTCAGCGAACCATTGCAACAGAGAGGTCCGAAGAAGATTCTGGCAGCCAAAGCTGC
GAGCAAGCCCCAGCAGGAGGAGGCGGAGGTGGAGGGAGTGACTCAGAAGCCGAATCTTCC
CAGTCCAGCTTAGATCTAAGGAGAGAGGGGTCACTTTCTCCAGTGAACTCACAAAAAATC
ACCTTGCTGCTGCAGTCCCCAGCGGTCAAGTTCATCACCAACCCCGAGTTCTTCACTGTG
CTACACGCCAATTATAGTGCCTACCGAGTCTTCACCAGTAGCACCTGCTTAAAGCACATG
ATTCTGAAAGTCCGACGGGATGCTCGCAATTTTGAACGCTACCAGCACAACCGGGACTTG
GTGAATTTCATCAACATGTTCGCAGACACTCGGCTGGAACTGCCCCGGGGCTGGGAGATC
AAAACGGACCAGCAGGGAAAGTCTTTTTTCGTGGACCACAACAGTCGAGCTACCACTTTC
ATTGACCCCCGAATCCCTCTTCAGAACGGTCGTCTTCCCAATCATCTAACTCACCGACAG
CACCTCCAGAGGCTCCGAAGTTACAGCGCCGGAGAGGCCTCAGAAGTTTCTAGAAACAGA
GGAGCCTCTTTACTGGCCAGGCCAGGACACAGCTTAGTAGCTGCTATTCGAAGCCAACAT
CAACATGAGTCATTGCCACTGGCATATAATGACAAGATTGTGGCATTTCTTCGCCAGCCA
AACATTTTTGAAATGCTGCAAGAGCGTCAGCCAAGCTTAGCAAGAAACCACACACTCAGG
GAGAAAATCCATTACATTCGGACTGAGGGTAATCACGGGCTTGAGAAGTIGTCCTGTGAT
GCGGATCTGGTCATTTTGCTGAGTCTCTTTGAAGAAGAGATTATGTCCTACGTCCCCCTG
CAGGCTGCCTTCCACCCTGGGTATAGCTTCTCTCCCCGATGTTCACCCTGTTCTTCACCT
CAGAACTCCCCAGGTTTACAGAGAGCCAGTGCAAGAGCCCCTTCCCCCTACCGAAGAGAC
TTTGAGGCCAAGCTCCGCAATTTCTACAGAAAACTGGAAGCCAAAGGATTTGGTCAGGGT
CCGGGGAAAATTAAGCTCATTATTCGCCGGGATCATTTGTTGGAGGGAACCTTCAATCAG
GTGATGGCCTATTCGCGGAAAGAGCTCCAGCGAAACAAGCTCTACGTCACCTTTGTTGGA
GAGGAGGGCCTGGACTACAGTGGCCCCTCGCGGGAGTTCTTCTTCCTTCTGTCTCAGGAG
CTCTTCAACCCTTACTATGGACTCTTTGAGTACTCGGCAAATGATACTTACACGGTGCAG
ATCAGCCCCATGTCCGCATTTGTAGAAAACCATCTTGAGTGGTTCAGGTTTAGCGGTCGC
ATCCTGGGTCTGGCTCTGATCCATCAGTACCTTCTTGACGCTTTCTTCACGAGGCCCTTC
TACAAGGCACTCCTGAGACTGCCCTGTGATTTGAGTGACCTGGAATATTTGGATGAGGAA
TTCCACCAGAGTTTGCAGTGGATGAAGGACAACAACATCACAGACATCTTAGACCTCACT
TTCACTGTTAATGAAGAGGTTTTTGGACAGGTCACGGAAAGGGAGTTGAAGTCTGGAGGA
GCCAACACACAGGTGACGGAGAAAAACAAGAAGGAGTACATCGAGCGCATGGTGAAGTGG
CGGGTGGAGCGCGGCGTGGTACAGCAGACCGAGGCGCTGGTGCGCGGCTTCTACGAGGTT
GTAGACTCGAGGCTGGTGTCCGTGTTTGATGCCAGGGAGCTGGAGCTGGTGATAGCTGGC
ACCGCGGAAATCGACCTAAATGACTGGCGGAATAACACTGAGTACCGGGGAGGTTACCAC
GATGGGCATCTTGTGATCCGCTGGTTCTGGGCTGCGGTGGAGCGCTTCAATAATGAGCAG
AGGCTGAGATTACTGCAGTTTGTCACGGGAACATCCAGCGTGCCCTACGAAGGCTTCGCA
GCCCTCCGTGGGAGCAATGGGCTTCGGCGCTTCTGCATAGAGAAATGGGGGAAAATTACT
TCTCTCCCCAGGGCACACACATGCTTCAACCGACTGGATCTTCCACCGTATCCCTCGTAC
TCCATGTTGTATGAAAAGCTGTTAACAGCAGTAGAGGAAACCAGCACCTTTGGACTTGAG
TGAGGACATGGAACCTCGCCTGACATTTTCCTGGCCAGTGACATCACCCTTCCTGGGATG
ATCCCCTTTTCCCTTTCCCTTAATCAACTCTCCTTTGATTTTGGTATTCCATGATTTTTA
TTTTCAAAC
Human NEDL2 nucleic acid sequence (uniprot.org/uniprot/Q9P2P5).
(SEQ ID NO: 56)
AGAGTTCCATCAGAGCCTGCAGTGGATGAAAGACAATGATATCCATGACATCCTAGACCT
CACGTTCACTGTGAACGAAGAAGTATTTGGGCAGATAACTGAACGAGAATTAAAGCCAGG
GGGTGCCAATATCCCAGTTACAGAGAAGAACAAGAAGGAGTACATCGAGAGGATGGTGAA
GTGGAGGATTGAGAGGGGTGTTGTACAGCAAACAGAGAGCTTAGTGCGTGGCTTCTATGA
GGTGGTGGATGCCAGGCTGGTATCTGTTTTTGATGCAAGAGAACTGGAATTGGTCATCGC
AGGCACAGCTGAAATAGACCTAAGTGATTGGAGAAACAACACAGAATATAGAGGAGGATA
CCATGACAATCATATTGTAATTCGGTGGTTCTGGGCTGCAGTGGAAAGATTCAACAATGA
ACAACGACTAAGGTTGTTACAGTTTGTTACAGGCACATCCAGCATTCCCTATGAAGGATT
TGCTTCACTCCGAGGGAGTAACGGCCCAAGAAGATTCTGTGTGGAGAAATGGGGGAAAAT
CACTGCTCTTCCCAGAGCGCATACATGTTTTAACCGTCTGGATCTGCCTCCCTACCCATC
CTTTTCCATGCTTTATGAAAAACTGTTGACAGCAGTTGAAGAAACCAGTACTTTTGGACT
TGAGTGACCTGGAAGCTGAATGCCCATCTCTGTGGACAGGCAGTTTCAGAAGCTGCCTTC
TAGAAGAATGATTGAACATTGGAAGTTTCAAGAGGATGCTTCCTTTAGGATAAAGCTACG
TGCTGTTGTTTTCCAGGAACAAGTGCTCTGTCACATTTGGGGACTGGAGATGAGTCCTCT
TGGAAGGATTTGGGTGAGCTTGATGCCCAGGGAACAACCCAACCGTCTTTCAATCAACAG
TTCTTGACTGCCAAACTTTTTCCATTTGTTATGTTCCAAGACAAAGATGAACCCATACAT
GATCAGCTCCACGGTAATTTTTAGGGACTCAGGAGAATCTTGAAACTTACCCTTGAACGT
GGTTCAAGCCAAACTGGCAGCATTTGGCCCAATCTCCAAATTAGAGCAAGTTAAATAATA
TAATAAAAGTAAATATATTTCCTGAAAGTACATTCATTTAAGCCCTAAGTTATAACAGAA
TATTCATTTCTTGCTTATGAGTGCCTGCATGGTGTGCACCATAGGTTTCCGCTTTCATGG
GACATGAGTGAAAATGAAACCAAGTCAATATGAGGTACCTTTACAGATTTGCAATAAGAT
GGTCTGTGACAATGTATATGCAAGTGGTATGTGTGTAATTATGGCTAAAGACAAACCATT
ATTCAGTGAATTACTAATGACAGATTTTATGCTTTATAATGCATGAAAACAATTTTAAAA
TAACTAGCAATTAATCACAGCATATCAGGAAAAAGTACACAGTGAGTTCTGTTTATTTTT
TGTAGGCTCATTATGTTTATGTTCTTTAAGATGTATATAAGAACCTACTTATCATGCTGT
ATGTATCACTCATTCCATTTTCATGTTCCATGCATACTCGGGCATCATGCTAATATGTAT
CCTTTTAAGCACTCTCAAGGAAACAAAAGGGCCTTTTATTTTTATAAAGGTAAAAAAAAT
TCCCCAAATATTTTGCACTGAATGTACCAAAGGTGAAGGGACATTACAATATGACTAACA
GCAACTCCATCACTTGAGAAGTATAATAGAAAATAGCTTCTAAATCAAACTTCCTTCACA
GTGCCGTGTCTACCACTACAAGGACTGTGCATCTAAGTAATAATTTTTTAAGATTCACTA
TATGTGATAGTATGATATGCATTTATTTAAAATGCATTAGACTCTCTTCCATCCATCAAA
TACTTTACAGGATGGCATTTAATACAGATATTTCGTATTTCCCCCACTGCTTTTTATTTG
TACAGCATCATTAAACACTAAGCTCAGTTAAGGAGCCATCAGCAACACTGAAGAGATCAG
TAGTAAGAATTCCATTTTCCCTCATCAGTGAAGACACCACAAATTGAAACTCAGAACTAT
ATTTCTAAGCCTGCATTTTCACTGATGCATAATTTTCTTATTAATATTAAGAGACAGTTT
TTCTATGGCATCTCCAAAACTGCATGACATCACTAGTCTTACTTCTGCTTAATTTTATGA
GAAGGTATTCTTCATTTTAATTGCTTTTGGGATTACTCCACATCTTTGTTTATTTCTTGA
CTAATCAGATTTTCAATAGAGTGAAGTTAAATTGGGGGTCATAAAAGCATTGGATTGACA
TATGGTTTGCCAGCCTATGGGTTTACAGGCATTGCCCAAACATTTCTTTGAGATCTATAT
TTATAAGCAGCCATGGAATTCCTATTATGGGATGTTGGCAATCTTACATTTTATAGAGGT
CATATGCATAGTTTTCATAGGTGTTTTGTAAGAACTGATTGCTCTCCTGTGAGTTAAGCT
ATGTTTACTACTGGGACCCTCAAGAGGAATACCACTTATGTTACACTCCTGCACTAAAGG
CACGTACTGCAGTGTGAAGAAATGTTCTGAAAAAGGGTTATAGAAATCTGGAAATAAGAA
AGGAAGAGCTCTCTGTATTCTATAATTGGAAGAGAAAAAAAGAAAAACTTTTAACTGGAA
ATGTTAGTTTGTACTTATTGATCATGAATACAAGTATATATTTAATTTTGCAAAAAAAAA
AAAAAAAAAAAAAAG

Some aspects of this invention provide expression constructs that encode any of the proteins, nucleic acids, such as RNAs, or fusions thereof described herein.

Nucleic acids encoding any of the proteins and/or nucleic acid (including RNA) described herein, may be in any number of nucleic acid “vectors” known in the art. As used herein, a “vector” means any nucleic acid or nucleic acid-bearing particle, cell, or organism capable of being used to transfer a nucleic acid into a host cell. The term “vector” includes both viral and nonviral products and means for introducing nucleic acid into a cell. A “vector” can be used in vitro, ex vivo, or in vivo. Non-viral vectors include plasmids, cosmids, artificial chromosomes (e.g., bacterial artificial chromosomes or yeast artificial chromosomes) and can comprise liposomes, electrically charged lipids (cytofectins), DNA-protein complexes, and biopolymers, for example. Viral vectors include retroviruses, lentiviruses, adeno-associated virus, pox viruses, baculovirus, reoviruses, vaccinia viruses, herpes simplex viruses, Epstein-Barr viruses, and adenovirus vectors, for example. Vectors can also comprise the entire genome sequence or recombinant genome sequence of a virus. A vector can also comprise a portion of the genome that comprises the functional sequences for the production of a virus capable of infecting, entering, or being introduced to a cell to deliver nucleic acid therein.

Expression of any of the proteins and/or nucleic acid (including RNA) described herein, may be controlled by any regulatory sequence (e.g., a promoter sequence) known in the art. Regulatory sequences, as described herein, are nucleic acid sequences that regulate the expression of a nucleic acid sequence. A regulatory or control sequence may include sequences that are responsible for expressing a particular nucleic acid or may include other sequences, such as heterologous, synthetic, or partially synthetic sequences. The sequences can be of eukaryotic, prokaryotic, or viral origin that stimulate or repress transcription of a gene in a specific or non-specific manner and in an inducible or non-inducible manner. Regulatory or control regions may include origins of replication, RNA splice sites, introns, chimeric or hybrid introns, promoters, enhancers, transcriptional termination sequences, poly A sites, locus control regions, signal sequences that direct the polypeptide into the secretory pathways of the target cell. A heterologous regulatory region is a regulatory region not naturally associated with the expressed nucleic acid it is linked to. Included among the heterologous regulatory regions are regulatory regions from a different species, regulatory regions from a different gene, hybrid regulatory sequences, and regulatory sequences that do not occur in nature, but which are designed by one of ordinary skill in the art.

The term “operably linked” refers to an arrangement of sequences or regions wherein the components are configured so as to perform their usual or intended function. Thus, a regulatory or control sequence operably linked to a coding sequence is capable of affecting the expression of the coding sequence. The regulatory or control sequences need not be contiguous with the coding sequence, so long as they function to direct the proper expression or polypeptide production. Thus, for example, intervening untranslated but transcribed sequences can be present between a promoter sequence and the coding sequence and the promoter sequence can still be considered operably linked to the coding sequence. A promoter sequence, as described herein, is a DNA regulatory region a short distance from the 5′ end of a gene that acts as the binding site for RNA polymerase. The promoter sequence may bind RNA polymerase in a cell and/or initiate transcription of a downstream (3′ direction) coding sequence. The promoter sequence may be a promoter capable of initiating transcription in prokaryotes or eukaryotes. Some non-limiting examples of eukaryotic promoters include the cytomegalovirus (CMV) promoter, the chicken j-actin (CBA) promoter, and a hybrid form of the CBA promoter (CBh).

### Cells Producing Microvesicles Containing Payload

A microvesicle-producing cell of the present invention may be a cell containing any of the expression constructs, any of the fusion proteins, or any of the payloads of molecules (e.g., biological molecules, small molecules, proteins, and nucleic acids (e.g., DNA, RNA), DNA plasmids siRNA, shRNA, mRNA) described herein. For example, an inventive microvesicle-producing cell may contain one or more recombinant expression constructs encoding (1) an ARRDC1 protein, or PSAP (SEQ ID NO: 1) motif-containing variant thereof and (2) an RNA binding protein (e.g., a Tat protein), that is associated with the ARRDC1 protein, or PSAP (SEQ ID NO: 1) motif-containing variant thereof. In some embodiments, a microvesicle-producing cell may contain one or more recombinant expression constructs encoding (1) an ARRDC1 protein, or PSAP (SEQ ID NO: 1) motif-containing variant thereof, and (2) a payload protein, such as a RNA binding protein fused to at least one WW domain, or variant thereof, under the control of a heterologous promoter. In certain embodiments, the expression construct in the microvesicle producing cell encodes a payload protein with one or more WW domains or variants thereof. In certain embodiments, an expression construct in the microvesicle producing cell encodes a RNA that associates with (e.g., binds specifically) an RNA binding protein, for example a therapeutic RNA.

Any of the expression constructs, described herein, may be stably inserted into the genome of the cell. In some embodiments, the expression construct is maintained in the cell, but not inserted into the genome of the cell. In some embodiments, the expression construct is in a vector, for example, a plasmid vector, a cosmid vector, a viral vector, or an artificial chromosome. In some embodiments, the expression construct further comprises additional sequences or elements that facilitate the maintenance and/or the replication of the expression construct in the microvesicle-producing cell, or that improve the expression of the fusion protein in the cell. Such additional sequences or elements may include, for example, an origin of replication, an antibiotic resistance cassette, a polyA sequence, and/or a transcriptional isolator. Some expression constructs suitable for the generation of microvesicle producing cells according to aspects of this invention are described elsewhere herein. Methods and reagents for the generation of additional expression constructs suitable for the generation of microvesicle producing cells according to aspects of this invention will be apparent to those of skill in the art based on the present disclosure. In some embodiments, the microvesicle producing cell is a mammalian cell, for example, a mouse cell, a rat cell, a hamster cell, a rodent cell, or a nonhuman primate cell. In some embodiments, the microvesicle producing cell is a human cell.

One skilled in the art may employ conventional techniques, such as molecular or cell biology, virology, microbiology, and recombinant DNA techniques. Exemplary techniques are explained fully in the literature. For example, one may rely on the following general texts to make and use the invention: Sambrook et al., Molecular Cloning: A Laboratory Manual, Second Edition (1989) Cold Spring Harbor Laboratory Press, Cold Spring Harbor, New York, and Sambrook et al., Third Edition (2001); DNA Cloning: A Practical Approach, Volumes I and II (D. N. Glover ed. 1985); Oligonucleotide Synthesis (M. J. Gaited. 1984); Nucleic Acid Hybridization (B. D. Hames & S. J. Higgins eds. (1985)); Transcription and Translation Hames & Higgins, eds. (1984); Animal Cell Culture (RI. Freshney, ed. (1986)); Immobilized Cells And Enzymes (IRL Press, (1986)); Gennaro et al., (eds.) Remington's Pharmaceutical Sciences, 18th edition; B. Perbal, A Practical Guide To Molecular Cloning (1984); F. M. Ausubel et al., (eds.), Current Protocols in Molecular Biology, John Wiley & Sons, Inc. (updates through 2001), Coligan et al., (eds.), Current Protocols in Immunology, John Wiley & Sons, Inc. (updates through 2001); W. Paul et al., (eds.) Fundamental Immunology, Raven Press; E. J. Murray et al., (ed.) Methods in Molecular Biology: Gene Transfer and Expression Protocols, The Humana Press Inc. (1991)(especially vol. 7); and J. E. Celis et al., Cell Biology: A Laboratory Handbook, Academic Press (1994).

### Delivery of ARMMs Containing Payload Molecules

The inventive microvesicles (e.g., ARMMs containing any of the expression constructs and/or any of the payload of molecules (e.g., biological molecules, small molecules, proteins, and nucleic acids (e.g., DNA, RNA), DNA plasmids, siRNA, shRNA, mRNA)), may optionally further comprise a targeting moiety. The targeting moiety may be used to target the delivery of ARMMs to specific cell types, resulting in the release of the contents of the ARMM into the cytoplasm of the specific targeted cell type. A targeting moiety may be a viral envelope protein that normally functions to aid viral attachment and entry into cells. The viral envelope protein may allow for the targeting of cells of the CNS. Viral envelope proteins include, but are not limited to, vesicular stomatitis virus G protein (VSV-G; Genbank Accession and Version Number: AJ318514.1) or rabies virus glycoprotein (RVG; Genbank Accession and Version Number: M38452.1). VSV-G protein, facilitates viral entry by mediating viral attachment to an LDL receptor (LDLR) or an LDLR family member present on a target cell. Subsequent to binding, the VSV-G-LDLR complex is rapidly endocytosed and proceeds to mediate the fusion of the viral envelope with the endosomal membrane. VSV-G enters the cell through partially clathrin-coated vesicles; virus-containing vesicles contain more clathrin and clathrin adaptor than conventional vesicles. VSV-G is a common coat protein for vector expression systems used to introduce genetic material into in vitro systems or animal models, mainly because of its extremely broad tropism. RVG is a trimeric and surface-exposed viral coat protein known to use the nicotinic acetylcholine receptor and the low affinity nerve growth factor receptor for viral entry. In some embodiments, a viral envelope protein (e.g., VSV-G, RVG) facilitates binding (e.g., targeting) of the ARMMs to cells of the nervous system such as the PNS and, more particularly, to Schwann cells.

A targeting moiety may selectively bind an antigen of the target nervous system cell. For example, the targeting moiety may be a membrane-bound immunoglobulin, an integrin, a receptor, a receptor ligand, an aptamer, a small molecule, or a variant thereof. Any number of cell surface proteins may also be included in an ARMM to facilitate the binding of an ARMM to a target cell and/or to facilitate the uptake of an ARMM into a target nervous system cell. Integrins, receptor tyrosine kinases, G-protein coupled receptors, and membrane-bound immunoglobulins suitable for use with embodiments of this invention will be apparent to those of skill in the art and the invention is not limited in this respect. For example, in some embodiments, the integrin is an α1β1, α2β1, α4β1, α5β1, α6β1, αLβ2, αMβ2, αIIbβ3, αVβ3, αVβ5, αVβ6, or a α6β4 integrin. In some embodiments, the receptor tyrosine kinase is a an EGF receptor (ErbB family), insulin receptor, PDGF receptor, FGF receptor, VEGF receptor, HGF receptor, Trk receptor, Eph receptor, AXL receptor, LTK receptor, TIE receptor, ROR receptor, DDR receptor, RET receptor, KLG receptor, RYK receptor, or MuSK receptor. In some embodiments, the G-protein coupled receptor is a rhodopsin-like receptor, the secretin receptor, metabotropic glutamate/pheromone receptor, cyclic AMP receptor, frizzled/smoothened receptor, CXCR4, CCR5, or beta-adrenergic receptor.

Additional molecules, such as synthetic small molecules or natural products, can be modified to associate with an ARMM protein (e.g., TSG101 or ARRDC1) for the purpose of targeting. This association can facilitate their incorporation into ARMMs, which in turn can be used to deliver the molecule to a target cell. Incorporation of a cleavable linker may be used to allow the small molecule to be released upon delivery in a target cell. As a non-limiting example, a small molecule can be linked to biotin, thereby allowing it to associate with an ARRDC1 protein which is fused to a streptavidin. As another non-limiting example, a small molecule can be linked to synthetic high affinity ligand that specifically binds to a mutant form of FKBP12 such as FKBP12(F36V) (Yang, W., et al., “Investigating protein-ligand interactions with a mutant FKBP possessing a designed specificity pocket” J. Med. Chem., 43(6):1135-1142 (2000)), which will associate with an ARRDC1 protein which is fused to FKBP12(F36V). The association of the small molecule to an ARMM protein (e.g., TSG101 or ARRDC1), facilitates loading of the small molecule into the ARRDC1-containing ARMM.

Some aspects of this invention relate to the recognition that ARMMs are taken up by target cells (e.g., cells of the PNS including Schwann cells), and ARMM uptake results in the release of the contents of the ARMM into the cytoplasm of the target cells. In some embodiments, the payload is an agent that affects a desired change in the target cell, for example, a change in cell survival, proliferation rate, a change in differentiation stage, a change in a cell identity, a change in chromatin state, a change in the transcription rate of one or more genes, a change in the transcriptional profile, or a post-transcriptional change in gene compression of the target cell. It will be understood by those of skill in the art, that the agent to be delivered will be chosen according to the desired effect in the target cell.

In some embodiments, cells from a subject are obtained and a payload is delivered to the cells by a system or method provided herein ex vivo. In some embodiments, the treated cells are selected for those cells in which a desired gene is expressed or repressed. In some embodiments, treated cells carrying a desired payload protein or payload RNA are returned to the subject they were obtained from.

In some embodiments, the ARMMs comprising any of the fusion proteins, any of the binding RNAs, any of the payload RNAs, and/or any of the binding RNAs fused to any of the payload RNAs, described herein, further include a detectable label. Such ARMMs allow for the labeling of a target cell without genetic manipulation. Detectable labels suitable for direct delivery to target cells are known in the art, and include, but are not limited to, fluorescent proteins, fluorescent dyes, membrane-bound dyes, and enzymes, for example, membrane-bound or cytosolic enzymes, catalyzing the reaction resulting in a detectable reaction product. Detectable labels suitable according to some aspects of this invention further include membrane-bound antigens, for example, membrane-bound ligands that can be detected with commonly available antibodies or antigen binding agents. Detectably labeled ARRMS find use in various diagnostic and analytical methods and applications.

In some embodiments, ARMMs are provided that comprise a payload RNA that encodes a transcription factor, a transcriptional repressor, a fluorescent protein, a kinase, a phosphatase, a protease, a ligase, a chromatin modulator, a recombinase, or the like. In some embodiments, ARMMs are provided that comprise a payload RNA (e.g., an siRNA, shRNA, mRNA) that inhibits the expression of a transcription factor, a transcriptional repressor, a fluorescent protein, a kinase, a phosphatase, a protease, a ligase, a chromatin modulator, or a recombinase. In some embodiments, the payload RNA is a therapeutic RNA. In some embodiments, the payload RNA is an RNA that affects a change in the state or identity of a target cell. For example, in some embodiments, the payload RNA encodes a reprogramming factor. Suitable transcription factors, transcriptional repressors, fluorescent proteins, kinases, phosphatases, proteases, ligases, chromatin modulators, recombinases, and reprogramming factors may be encoded by a payload RNA that is associated with a binding RNA to facilitate their incorporation into ARMMs and their function may be tested by any methods that are known to those skilled in the art, and the invention is not limited in this respect.

Methods for isolating the ARMMs described herein are also provided. One exemplary method includes collecting the culture medium, or supernatant, of a cell culture comprising microvesicle-producing cells. In some embodiments, the cell culture comprises cells obtained from a subject, for example, cells suspected to exhibit a pathological phenotype, for example, a hyperproliferative phenotype. In some embodiments, the cell culture comprises genetically engineered cells producing ARMMs, for example, cells expressing a recombinant ARMM protein, for example, a recombinant ARRDC1 or TSG101 protein, such as an ARRDC1 or TSG101 protein, optionally fused to an RNA binding protein (e.g., a Tat protein) or variant thereof. In some embodiments, the supernatant is pre-cleared of cellular debris by centrifugation, for example, by two consecutive centrifugations of increasing G value (e.g., 500G and 2000G). In some embodiments, the method comprises passing the supernatant through a 0.2 μm filter, eliminating all large pieces of cell debris and whole cells. In some embodiments, the supernatant is subjected to ultracentrifugation, for example, at 120,000G for 2 hours, depending on the volume of centrifugate. The pellet obtained comprises microvesicles. In some embodiments, exosomes are depleted from the microvesicle pellet by staining and/or sorting (e.g., by FACS or MACS) using an exosome marker as described herein. Isolated or enriched ARMMs can be suspended in culture media or a suitable buffer, as described herein.

### Methods of Microvesicle-Mediated Delivery of Payload to Cells of the Nervous System

Some aspects of this invention provide a method of delivering an agent (e.g., a therapeutic agent or agents) to a target cell of the nervous system. The target cell can be contacted with an ARMM in different ways. For example, a target cell may be contacted directly with an ARMM as described herein, or with an isolated ARMM from a microvesicle producing cell. The contacting can be done in vitro by administering the ARMM to the target cell in a culture dish, or in vivo by administering the ARMM to a subject (e.g., parenterally or non-parenterally). In some embodiments, an ARMM is produced from a cell obtained from a subject. In some embodiments, the ARMM that was produced from a cell that was obtained from the subject is administered to the subject from which the ARMM producing cell was obtained. In some embodiments, the ARMM that was produced from a cell that was obtained from the subject is administered to a subject different from the subject from which the ARMM producing cell was obtained. As one example, a cell may be obtained from a subject and engineered to express one or more of the constructs provided herein (e.g., engineered to express a payload RNA associated with a binding RNA, an ARRDC1 protein, an ARRDC1 protein fused to an RNA binding protein, and/or an RNA binding protein fused to a WW domain). The cell obtained from the subject and engineered to express one or more of the constructs provided herein may be administered to the same subject, or a different subject, from which the cell was obtained. Alternatively, the cell obtained from the subject and engineered to express one or more of the constructs provided herein produces ARMMs, which may be isolated and administered to the same subject form which the cell was obtained or administered to a different subject from which the cell was obtained.

Alternatively, a target cell of the nervous system can be contacted with a microvesicle producing cell as described herein, for example, in vitro by co-culturing the target cell and the microvesicle producing cell, or in vivo by administering a microvesicle producing cell to a subject harboring the target cell. Accordingly, the method may include contacting the target cell with a microvesicle, for example, an ARMM containing any of the payload to be delivered, as described herein. The target cell may be contacted with a microvesicle-producing cell, as described herein, or with an isolated microvesicle that has a lipid bilayer, an ARRDC1 protein or variant thereof, a payload and optionally a viral envelope protein.

It should be appreciated that the target cell of the nervous system may be of any origin, for example, from an organism. In some embodiments, the target cell is a mammalian cell. Some non-limiting examples of a mammalian cell include, without limitation, a mouse cell, a rat cell, a hamster cell, a rodent cell, and a nonhuman primate cell. In some embodiments, the target cell is a human cell. It should also be appreciated that the target cell may be of any cell type of the nervous system. In other cases, the target cell may be any differentiated cell type found in a subject. In some embodiments, the target cell is a cell in vitro, and the method includes administering the microvesicle to the cell in vitro, or co-culturing the target cell with the microvesicle-producing cell in vitro. In some embodiments, the target cell is a cell in a subject, and the method comprises administering the microvesicle or the microvesicle-producing cell to the subject. In some embodiments, the subject is a mammalian subject, for example, a rodent, a mouse, a rat, a hamster, or a non-human primate. In some embodiments, the subject is a human subject.

In some embodiments, the target cell is a pathological cell. In some embodiments, the target cell is a cancer cell. In some embodiments, the microvesicle is associated with a binding agent that selectively binds an antigen on the surface of the target cell. In some embodiments, the antigen of the target cell is a cell surface antigen. In some embodiments, the binding agent is a membrane-bound immunoglobulin, an integrin, a receptor, a receptor ligand, or a lectin, among other suitable candidate molecules and moieties. Suitable surface antigens of target cells (e.g., cells of the nervous system), for example of specific target cell types, e.g., cancer cells, are known to those of skill in the art, as are suitable binding agents that specifically bind such antigens. Methods for producing membrane-bound binding agents, for example, membrane-bound immunoglobulins, membrane-bound antibodies, or antibody fragments that specifically bind a surface antigen expressed on the surface of cancer cells, are also known to those of skill in the art. The choice of the binding agent will depend, of course, on the identity or the type of target cell. Cell surface antigens specifically expressed on various types of nervous system cells that can be targeted by ARMMs comprising membrane-bound binding agents will be apparent to those of skill in the art. It will be appreciated that the present invention is not limited in this respect.

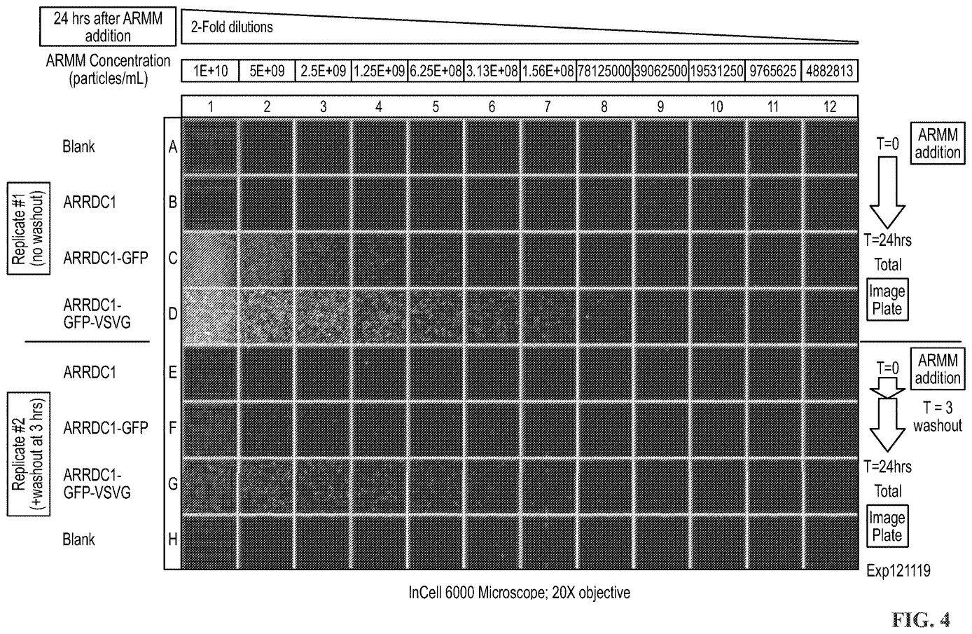

In some embodiments, the target cells of the nervous system include disease targets. A non-limiting example of genetic targets for ARMM therapeutics for CMTs include: PMP22 (e.g., in CMT1, CMT1A, CMT1E diseases), MPZ (e.g., in CMT1B, CMT3 diseases), NEFL, LITAF (e.g., in CMT1C disease), EGR2 (e.g., in CMT1D, CMT3, CNT4E diseases), GJB1 (e.g., in CMT1X disease), NEFL (e.g., in CMT1F disease), MFN2 (e.g., in CMT2A, [CMT2A2B] diseases), glycl RNA synthetase gene (in e.g., CMT2D disease), neurofilament light gene (e.g., CMT2E disease), HSPB1 (e.g., CMT2F disease), GDAP1 (e.g., CMT2K, CMT4A diseases), DYNC1H1 (e.g., CMT20 disease), LRSAM1 (e.g., CMT2P disease), IGHMBP2 (e.g., CMT2S disease), MORC2 (e.g., CMT2Z disease), defects on chromosome 11 (e.g., CMT4B disease), SH3TC2 (e.g., CMT4C disease), defects on chromosome 8 (e.g., CMT4D disease), PRX (e.g., CMT4F disease), and FIG. 4 (e.g., CMT4J disease), and other genetic targets. (See e.g., Li, J., et al., “The PMP22 Gene and Its Related Diseases,” Mol. Neirobiol., 47(2):673-698 (2013)).

A non-limiting example of genetic targets for ARMM therapeutics for Schwannomatosis and Schwannoma tumors and related indications include: Akt, NF2, LZTR1, PI3K, and SMARCB1. (See e.g., Fong, B., et al., “The molecular biology and novel treatments of vestibular schwannomas,” J. Neurosurg., 115(5):906-914 (2011); Agnihotri, S., et al., “The genomic landscape of schwannoma,” Nat. Genet., 48(11):1339-1348 (2016); Mansouri, S., et al., “Epigenomic, genomic, and transcriptomic landscape of schwannomatosis,” Acta Neuropathol., 141(1):101-116 (2021); and Yin, X., et al., “MiR-205 Inhibits Sporadic Vestibular Schwannoma Cell Proliferation by Targeting Cyclin-Dependent Kinase 14,” World Neurosurg., 147:e25-e31 (2021)).

For example, a non-limiting example of genetic targets for ARMM therapeutics for Alzheimer's disease (familial forms and late onset) and related indications include: APP, PSEN1, PSEN2, APOE (e2), APOE (e3), APOE (e4), ADAMTS4, HESX1, HS3ST1, HLA-DQB1, NYAP1, CNTNAP2, ECHDC3, ADAM10, APH1B, KAT8, ABI3, SCIMP, ACE, ALPK2, BHMG1, ADAMTS1, IQCK1, CLU, SORL1, ABCA7, TREM2, CD33, MS4A6A, CR1, EPHA1, HLA-DRB1, HLA-DRB5, IL1RAP, INPP5D, PLCG2, CD2AP, BIN1, RIN3, SLC24A4, PICALM, PTK2B, CASS4, ABI3, FERMT2, SPIl, MEF2C, ZCWPWT, NME8, CR1, and PICALM.

A non-limiting example of genetic targets for ARMM therapeutics for frontotemporal dementia/amyotrophic lateral sclerosis spectrum disorders and related indications include: MAPT, GRN, C90RF72, SOD1, FUS, UBQLN2, CHCHD10, SQSTM1, VCP, CHMP2B, TBK1, CTSD, CTSF, TRKA, ERBB4, EWSR1, TAF15, HNRNPA1, HNRNPA2B1, ATXN2, OPTN, ANG, SETX, DAO, PFN1, ALS2, VAPB, SIGMAR1, MATR3, NEK1, PFN1, TIA1, and TUBA4A.

A non-limiting example of genetic targets for ARMM therapeutics for Parkinson's disease and related indications include: SNCA, LRRK2, PARK7, PINK1, PRKN, DJ-1, VPS35, UCHL1, ATP13A2, and GBA1.

A non-limiting example of genetic targets for ARMM therapeutics for other neurological diseases genetic targets include: SMN1, SMN2, HTT, DMPK, FMR1, MECP2, CIC, TCF4, CNTNAP2, STXBP1, SHANK2, TSC1, TSC2, SPG11, SEPT9, PANK2, PLA2G6, C19orf12, FTL, MR1, SLC2A1, DRD2, GCH1, GCDH, PRKRA, SGCE, THAP1, TOR1A, TAF1, TIMM8A, ACTB, SLC6A3, DYNC1H1, YARS, MPZ, NEFL, ARHGEF10, LITAF, EGR2, MFN2, RAB7A, LMNA, GARS, HSPB1, GDAP1, HSPB8, DNM2, SH3TC2, MTMR2, SBF2, NDRG1, PRX, FGD4, FIG. 4, GJB1, PRPS1, CTDP1, GAN, BSCL2, WNK1, IKBKAP, NTRK1, NGF, SPTLC1, and TH.

A non-limiting example of genetic targets for ARMM therapeutics for pain disorders and related indications include: SCN9A, MC1R, and FAAH.

### Pharmaceutical Compositions

Other aspects of the present disclosure relate to pharmaceutical compositions comprising any of the ARMMs or microvesicle (e.g., ARMM) producing cells provided herein. The term “pharmaceutical composition,” as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue, system, or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include, but are not limited to: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C1 alcohols, such as ethanol; and (24) other non-toxic compatible substances employed in pharmaceutical formulations. Wetting agents, coloring agents, release agents, coating agents, sweetening agents, flavoring agents, perfuming agents, preservatives, antioxidants, and the like, can also optionally be present in a formulation. Terms such as “excipient”, “carrier”, “pharmaceutically acceptable carrier” or the like are used interchangeably herein.

In some embodiments, the pharmaceutical composition is formulated for delivery to a subject e.g., for delivering a payload protein or payload RNA (e.g., a payload RNA that expresses a tumor suppressor) to a cell. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., cell of the nervous system). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, the pharmaceutical composition is formulated in accordance with routine procedures is a composition adapted for intravenous or subcutaneous administration to a subject, e.g., a human. In some embodiments, the pharmaceutical composition for administration by injection is a solution in a sterile isotonic aqueous buffer. Where necessary, the pharmaceutical can also include a solubilizing agent and a local anesthetic such as lidocaine to ease pain at the site of the injection. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's solution or Hank's solution. In addition, the pharmaceutical composition can be in solid forms and re-dissolved or suspended immediately prior to use. Lyophilized forms are also contemplated.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing an ARMM or microvesicle producing cell of the invention and (b) a second container containing a pharmaceutically acceptable diluent (e.g., sterile water) for injection. The pharmaceutically acceptable diluent can be used, e.g., for reconstitution or dilution of the ARMM or microvesicle producing cell of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration. In this regard, state-specific and regional regulatory agencies are understood to include, but are not limited to, the U.S. Food and Drug Administration, the U.S. Department of Agriculture, the European Medicines Agency, the United Kingdom Medicines and Healthcare Products Regulatory Agency, the National Medical Products Administration, and the like.

In another aspect, an article of manufacture containing materials useful for treating the diseases described herein is included. In some embodiments, the article of manufacture comprises a container and a label. Suitable containers include, for example, bottles, vials, syringes, and test tubes. The containers may be formed from various materials such as glass or plastic. Suitable containers are further understood to include materials that are sufficiently non-reactive and protective of the contents therein. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating one or more diseases of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions and indications for acceptable, recognized, or permitted use(s).

### Kits, Vectors, Cells

Some aspects of this disclosure provide kits comprising a nucleic acid construct comprising a nucleotide sequence encoding one or more of any of the proteins (e.g., ARRDC1, and TSG101), fusion proteins and/or nucleic acids provided herein. In some embodiments, the nucleotide sequence encodes any of the proteins, fusion proteins, and/or RNAs provided herein. In some embodiments, the nucleotide sequence comprises a heterologous promoter that drives the expression of any of the proteins, fusion proteins, and/or RNAs provided herein.

Some aspects of this disclosure provide microvesicle (e.g., ARMM) producing cells comprising any of the proteins, fusion proteins, and nucleic acids (e.g., RNAs) provided herein. In some embodiments, the cells specifically comprise a nucleotide that encodes any of the proteins, fusion proteins, and/or RNAs provided herein. In some embodiments, the cells comprise any of the nucleotides or vectors provided herein. In some embodiments, the vector comprises one or more viral targeting or viral entry proteins (e.g., fusogen(s)).

It should be appreciated, however, that additional proteins, fusion proteins, and RNAs would be apparent to the skilled artisan based on the present disclosure and knowledge in the art.

The function and advantage of these and other embodiments of the present invention will be more fully understood from the Examples below. The following Examples are intended to illustrate the benefits of the present invention and to describe particular embodiments but are not intended to exemplify the full scope of the invention.

Accordingly, it will be understood that the Examples are not meant to limit the scope of the invention.

### EXAMPLES

### Example 1: ARMM Platform Development for CNS Disorders in Human Induced Pluripotent Stem Cells (iPSC) Models for Biological & Therapeutic Discovery and Development

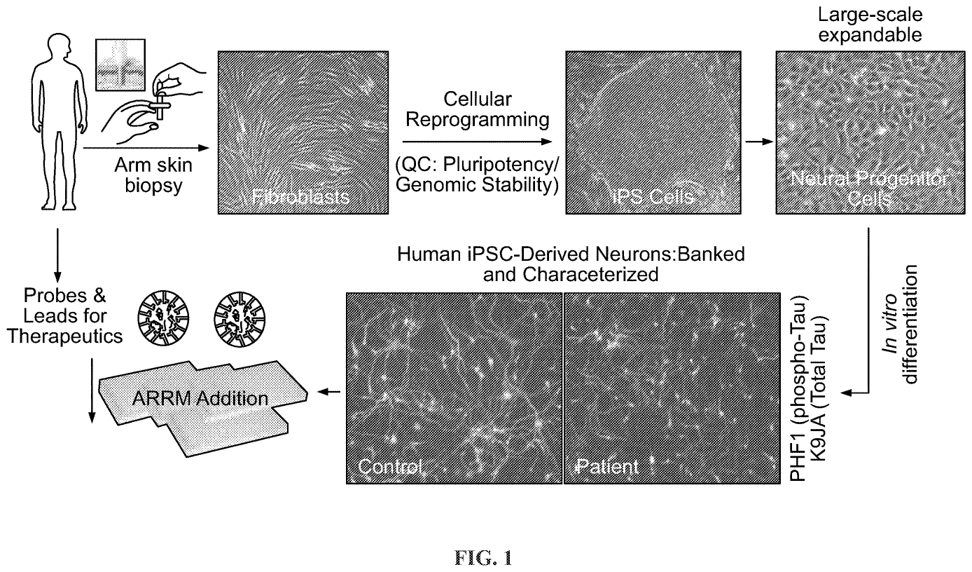

A methodology has been created wherein fibroblasts are isolated from a subject, by means of a skin biopsy (although other isolation techniques may be used), cells are reprogrammed into induced pluripotent stem cells (iPSCs) and directed to differentiate into neural progenitor cells (FIG. 1). iPSCs are cells derived from the skin or blood which have been reprogrammed back into an embryonic-like pluripotent state. This embryonic-like state enables the cells to be differentiated into additional types of cells on an as needed basis, providing a nearly unlimited source of any type of cell needed for therapeutic or research purposes (e.g., iPSC can be differentiated into neurons to treat or research neurological disorders). Neural progenitor cells are the progenitor cells of the CNS that give rise to many, if not all, of the glial and neuronal cell types that populate the CNS. Neural progenitor cells do not generate the non-neural cells also present in the CNS, such as immune system cells. The cells are allowed to differentiate in vitro into neuronal cells and are banked and characterized against a control group of neurons of the same species (in this case human) for tau protein expression using PHF1 (phosphorylated tau protein) and K9JA (total tau protein). Subsequently, ARRDC1-mediated microvesicles (ARMMs) containing target probes and therapeutic leads (e.g., small molecules, proteins, nucleic acids) are screened against the control and subject neuronal cells.

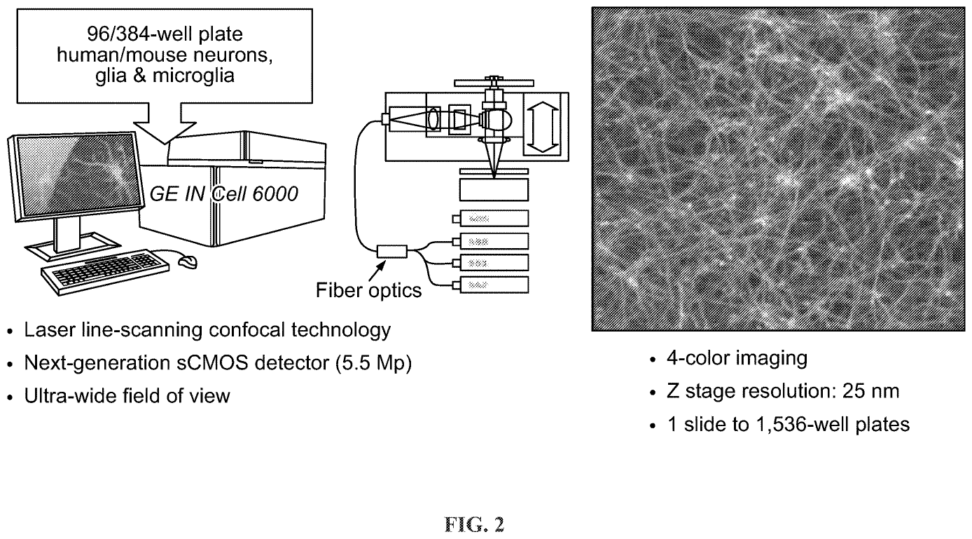

An imaging system has also been developed for analyzing the results of an ARMM-mediated payload screen. Automated confocal microscopy was used in an assay to analyze high-content single-cell level imaging (FIG. 2). Laser line-scanning confocal technology was used with a next-generation sCMOS detector (5.5 Mp) and an ultra-wide field of view. High-density 96-well plates were used to analyze human neural progenitor cells, neurons and glial cells with four channel imaging.

### Example 2: Payload and Target Cells Types for Platform Development

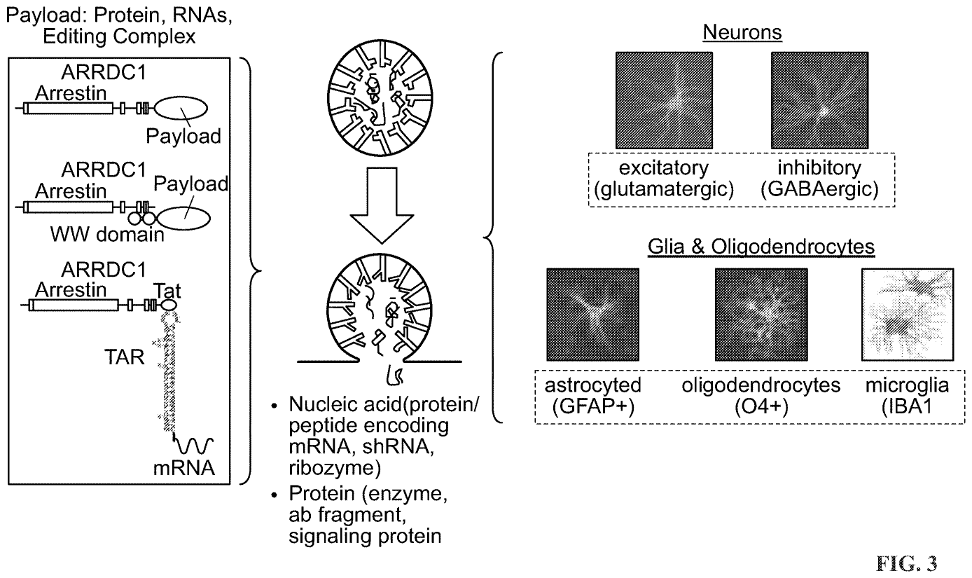

A platform has been developed for using ARRDC1-mediated microvesicles (ARMMs) for the delivery of payload molecules (e.g., biological molecules, such as proteins, nucleic acids (e.g., DNA, RNA, DNA plasmids, siRNA, mRNA), editing complexes, and small molecules to various nervous system cell types, such as cells of the CNS and PNS. The ARMMs are loaded with one of these molecules as the payload and used to deliver the payload to the cells of the nervous system, such as cells of the CNS (FIG. 3). For example, the molecule can either be directly linked to the ARRDC1 protein; the molecule can be associated with the ARRDC1 protein by fusing one or more WW domains to the molecule, which allows the molecule to associate with the PPXY (SEQ ID NO: 2) motif of ARRDC1; or the molecule can be associated with an ARRDC1-Tat fusion protein for delivery of TAR-payload RNA. Various molecular payloads are contemplated by the platform, including but not limited to proteins, peptides, DNAs, RNAs (e.g., mRNA, siRNA, shRNA, miRNA, ribozymes), antibody fragments, signaling proteins, editing complexes (e.g., CRISPR/Cas9, variants thereof), and small molecules. Further, various cells of the nervous system are targeted by the platform, including by not necessarily limited to cells of the CNS, including neurons, glia, oligodendrocytes, astrocytes, and microglia.

### Example 3: Use of Viral Envelope Proteins to Target Cells of the CNS

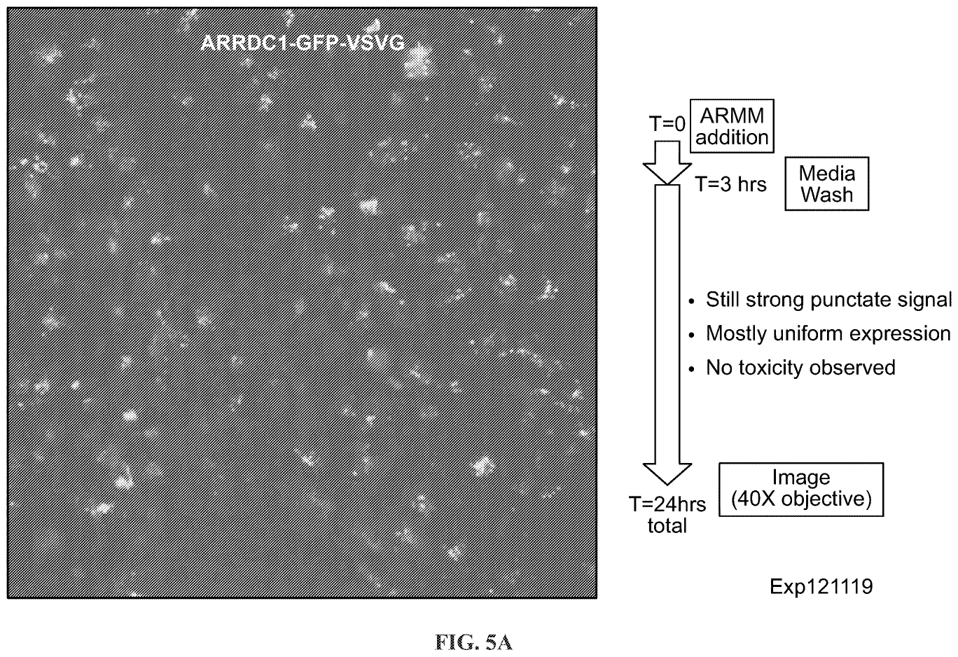

The viral envelope protein VSV-G was analyzed to determine if it could be used to enhance the uptake of ARMMs containing molecular payloads. ARMMs were added in various concentrations (as shown across the top of the plate) as four experimental sets, blank, ARRDC1, ARRDC1-GFP, and ARRDC1-GFP-VSV-G, for delivery to neural progenitor cells and incubated for 24 hours (24 h) (FIG. 4). The experiment was performed in two replicates, replicate 1 received no washout, whereas replicate 2 received a washout at hour 3. Imaging of both plates was performed after the incubation. As shown, the use of the VSV-G protein increased the delivery and expression of GFP in both replicates and across concentrations. Looking specifically at the expression of GFP in iPSC-derived neural progenitor cells with the ARRDC1-GFP-VSV-G delivery system with washout at hour 3, strong punctate signal was seen that was mostly uniform across cells, and the system exhibited no visible toxicity (FIG. 5A). In addition, imaging showed the subcellular localization of GFP delivered with an ARMM utilizing the VSV-G protein in human neurons isolated from iPSC-derived cerebral organoids (FIG. 5B).

### Example 4: ARMM-Mediated Delivery of mRNA Payloads in Human Neurons

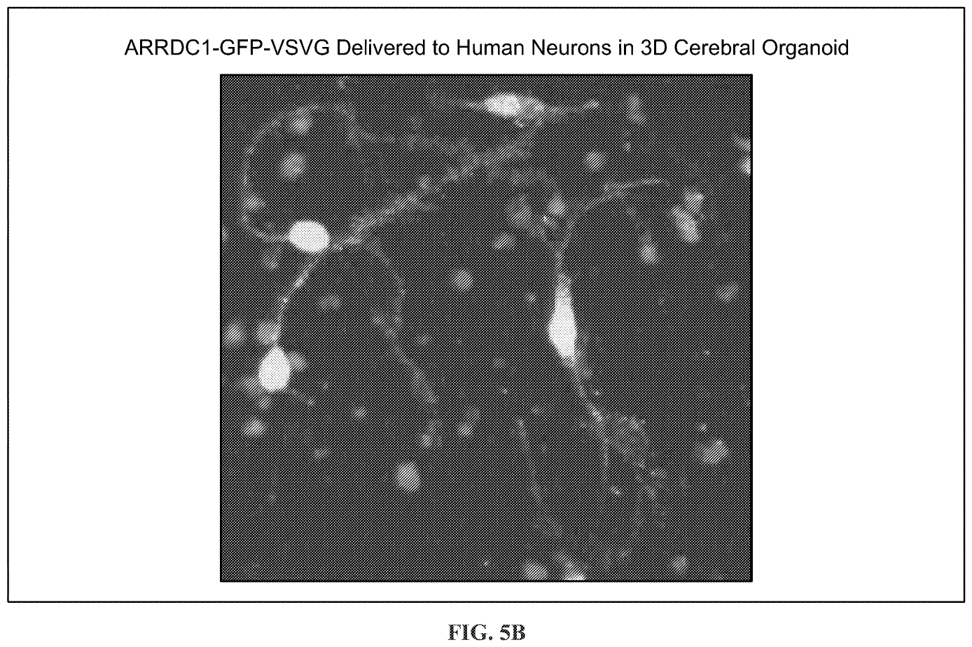

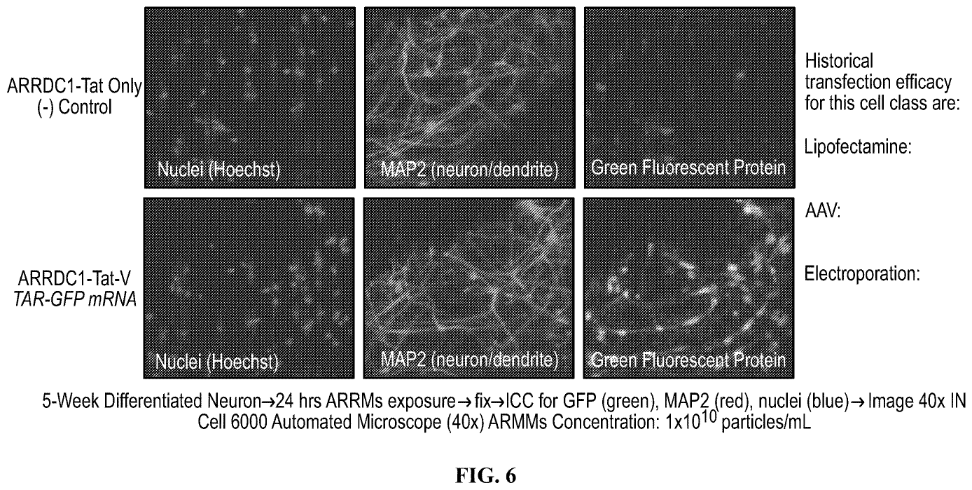

Payload delivery and success of protein translation were further evaluated (FIG. 6). ARRDC1-Tat (control) and ARRDC1-Tat-V with TAR-GFP mRNA as the payload RNA payload cargo (1×10¹⁰ particles per milliliter (particles/mL)) were introduced to neurons after being differentiated for 5 weeks. The cells were fixed 24 h after ARMM exposure and imaged using immunocytochemistry techniques. As shown, the ARMMs were successful at delivering the payload to the cells which was subsequently successfully translated into protein (FIGS. 6-7).

### Example 5: Targeting Multiple Neurogenetic Disorders Using ARMMs

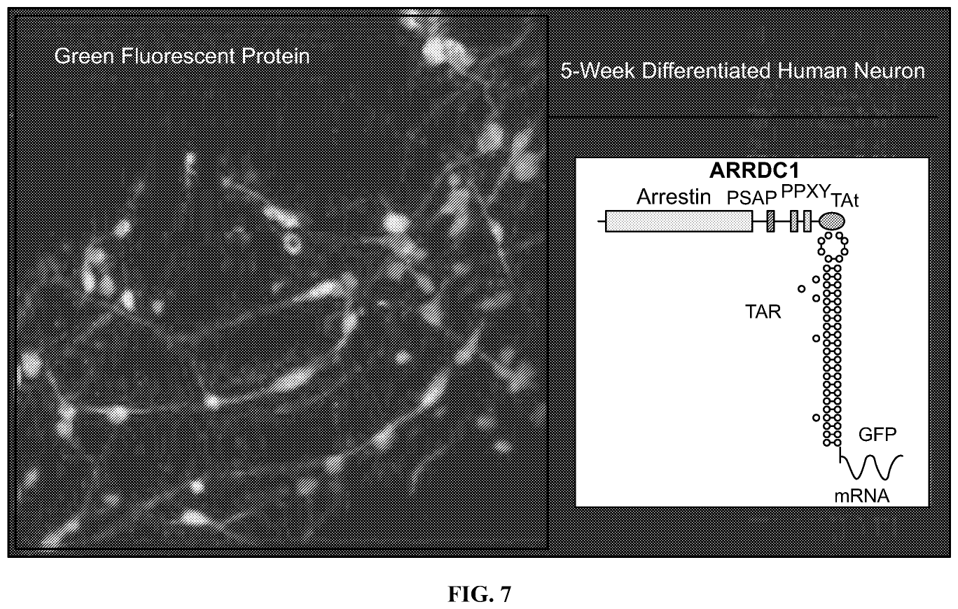

The ARMMs-mediated delivery technology can be adapted to different targets for both gain-of-function disorders (for example, but not limited to, due to mutations or dysfunction of MAPT, SNCA, HTT, ATXN2) and loss-of-function disorders (for example, but not limited to, due to mutations or dysfunction of GRN, GBA1, FMR1, MECP2, TCF4), and repeat expansion (for example, but not limited to, due to mutations in C9orf72) (FIG. 8, adapted from van der Zee & Van Broeckhoven, Nat. Rev. Neurol. 2014). Potential molecules for use as the payload include, but are not limited, shRNA, miRNAs, ribozymes, scFv PROTACs, editors (for example, nucleic acid editors (e.g., CRISPR/Cas9, and variants thereof)), and mRNA. Experiments can be conducted on patient iPSC-derived cells to optimize ARMM-mediated delivery of molecules, for example, shRNA/antisense RNA delivery to overcome the gain-of-function accumulation of defective tau and dipeptide repeat production with C9orf72 or dCas9 modified with a transcriptional activator (CRISPRa) or repressor (CRISPRi).

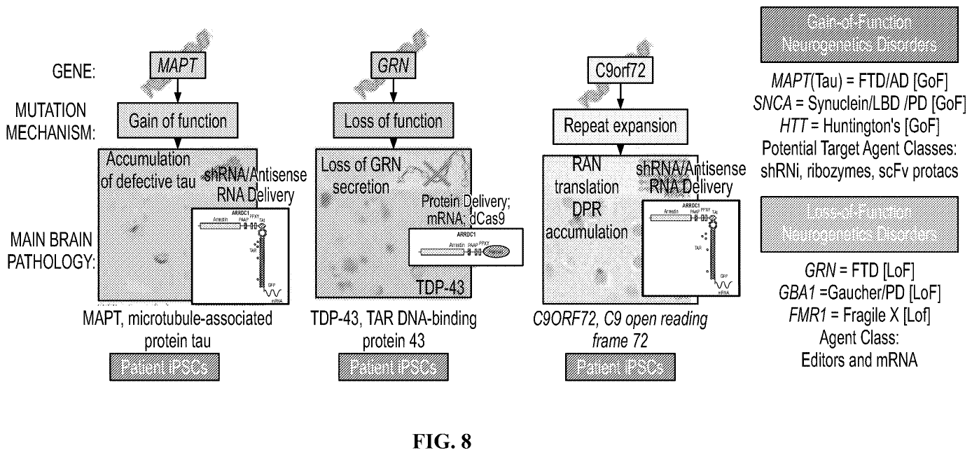

An example of the use of CRISPR editors that would be compatible with the ARMM-delivery technology are shown in FIG. 9. The schematic shows the use of dCas9 modified with a transcriptional activator (CRISPRa) with guide RNAs directed to the GRN locus to enhance the transcription of the GRN gene to overcome the loss-of-function of progranulin (PGRN) production due to GRN mutations. The graph shows the amount of PGRN in ng/ml per 110 μg total protein with the use of a mock sample, a dCas9-VPR sample, and a dCas9-VPR+2 GRN guide RNAs as determined by PGRN ELISA (R&D Quantikine).

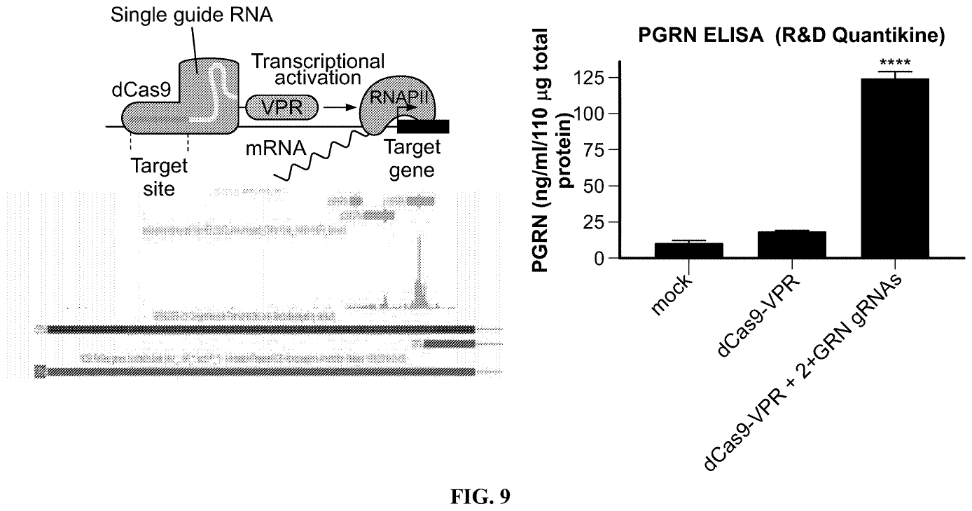

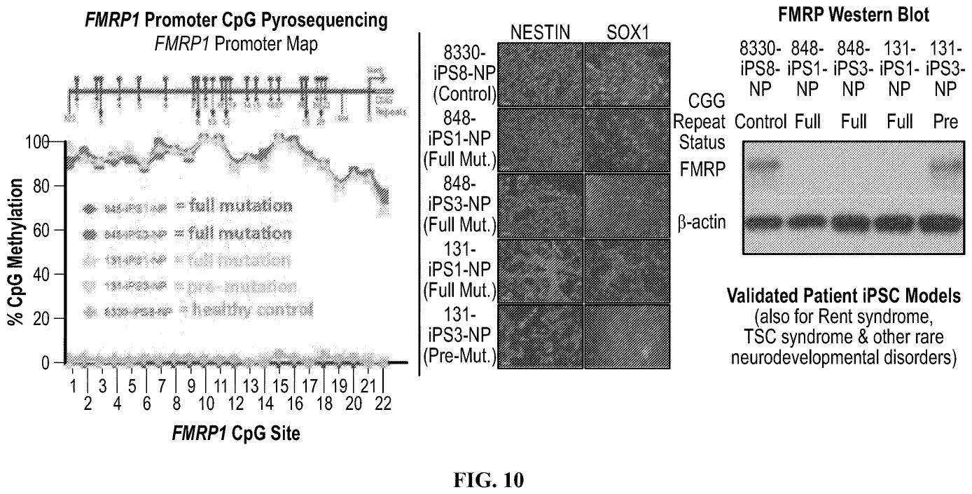

It is also possible that FMRP can be delivered to rescue Fragile X syndrome patient neurons using CNS-optimized ARMMs based upon our development of patient-derived iPSC models (FIGS. 10-11). For example (See, from Sheridan S. D., et al., “Epigenetic characterization of the FMR1 gene and aberrant neurodevelopment in human induced pluripotent stem cell models of fragile X syndrome.” PLoS One. 2011; 6(10):e26203), the graph shows the percentage of CpG methylation for each FMR1 CpG site with either full mutations (848-iPS1-NP, 848-iPS3-NP, 131-iPS1-NP), pre-mutation (131-iPS3-NP), or healthy control (8330-iPS8-NP) with a FMR1 promoter map provided. Elevated CpG methylation leads to epigenetic silencing of FMR1. The image shows NESTIN and SOX1 staining for each of the sample indicating the cells are neural progenitor cells. The Western blot show the amount of produced FMRP for each of the sample, with (i-actin as the control. FIG. 11 shows differentiation of cells with for 18 days resulting in post-mitotic neurons and glia. These cells were subjected to fixation and immunostaining as shown in the images. The observable phenotypic differences between the control and Fragile X patient lines with reduced FMRR expression provides a screenable assays using high-content imaging to optimize ARMM-based therapeutics. It is anticipated that similar screening methodology to optimize ARMM-based therapeutics will work for Rett syndrome due to mutations in MECP2 and similar iPSC models along with other rare neurodevelopmental disorders. (See e.g., Mellios, N., et al., “MeCP2-regulated miRNAs control early human neurogenesis through differential effects on ERK and AKT signaling,” Mol. Psychiatry, 23(4):1051-1065 (2018)).

### Example 6: Insertion of RVG into ARMMs

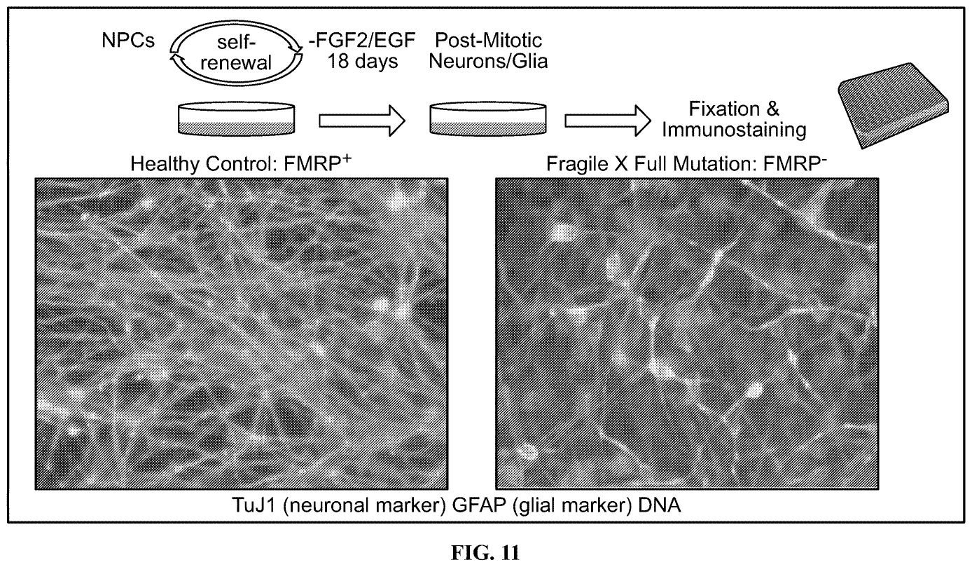

Using proteomics studies of ARMMs from multiple human cells, multiple proteins that are enriched in the ARMM vesicles were identified. Among the proteins were tetraspanins such as TSPAN14 and TSPAN6. These proteins have multiple extracellular loop regions that allow the insertion of “homing peptide” or other targeting moieties, thus enabling the potential targeting of ARMMs to specific cells/tissues. A fusion construct was developed, in which the rabies viral glycoprotein (RVG) peptide along with a HA tag was inserted into the second extracellular loop of TSPAN6 (FIG. 12A). Western blotting was performed on whole cell lysates and ARMMs using the antibodies directed the following targets: ARRDC1/GFP, TSPAN6/RVG/HA, CD9 and Vinculin. After transfection into HEK293 cells, Western blot analysis showed that TSPAN6-RVG-HA was robustly detected in ARMMs secreted from HEK293T cells (FIG. 12B).

### Example 7: Insertion of VSV-G into ARMMs

HEK293T cells (2×10⁶ cells/plate) were transfected with ARRDC1 (A1), or ARRDC1 along with TAR-GFP, in the presence or absence of VSV-G, in accordance with Table 1.

TABLE 1
Transfection information

| Plasmid | VSV-G |
|---|---|
| A1-Tat (5 μg/plate) | — |
| A1-Tat (3 μg/plate) + TAR-GFPmRNA (3 μg/plate) | — |
| A1-Tat (3 μg/plate) + TAR-GFPmRNA (3 μg/plate) | 0.5 ug/plate |

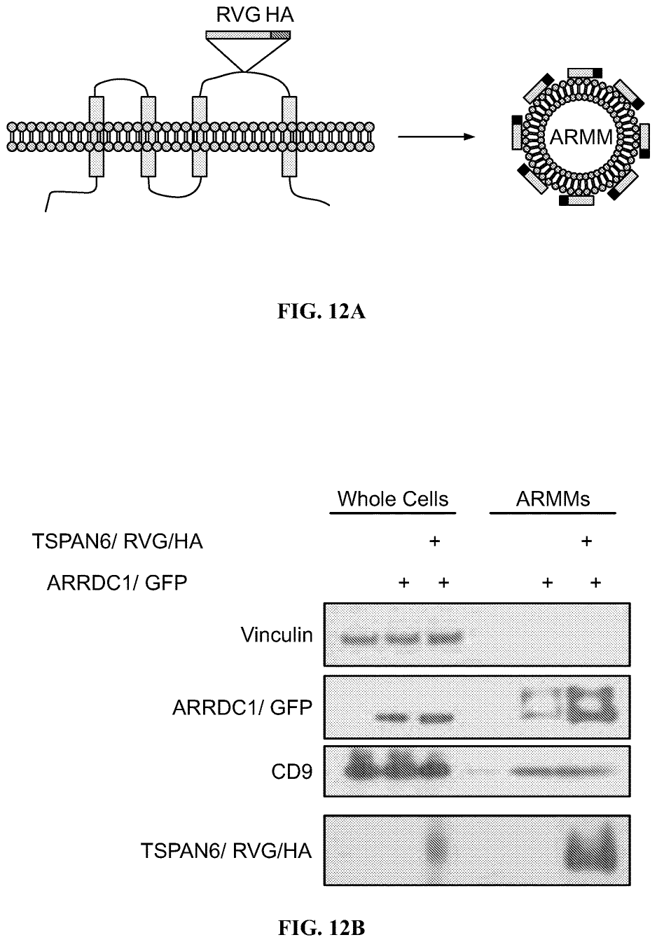

Extracellular vesicles were isolated using ultracentrifugation (FIG. 13A). Culture media was harvested 48 and 72 hours following transfection. The media was centrifuged at 3,000 g for 10 minutes and passed through a 0.22 μm filter. The supernatant was collected and centrifuged at 10,000 g for 10 minutes. The resulting supernatant was subjected to ultracentrifugation at 320,000 rpm for 2 hours. ARMMs were then resuspended in phosphate buffered saline (PBS). Western blotting was performed on whole cell lysates and ARMMs using antibodies directed to the following targets: unpurified ARRDC1 serum, VSV-G, CD9 and Vinculin (FIG. 13B). The results show that VSV-G was robustly detected in ARMMs. In addition, VSV-G appeared to increase the production of ARMMs, as indicated by the increased amount of ARRDC1 in the extracellular vesicle preparation.

### Example 8: ARRDC1-Mediated Delivery of Payloads to Cultured Human iPSC-Derived 3D Cerebral Organoids

Starting with human induced pluripotent stem cells (iPSCs), 3-dimensional (3D), cerebral tissue-like containing neurons from both deep and superficial cortical layers along with astrocytes were differentiated following published methodology. (Paşca, A. M, et al., “Functional cortical neurons and astrocytes from human pluripotent stem cells in 3D culture,” Nature Methods, 12(7):671-678 (2015)). After maturation en masse for 9 months in cerebral organoid maturation media (Neurobasal A, GlutaMax, B-27 supplement without vitamin A, penicillin/streptomycin), single organoids were isolated then exposed to ARRDC1-GFP-VSVG extracellular vesicles (14.3 mL) for either 24 hours or 48 hours in a 96-well plate format. Single organoids were then dissociated with Accutase and the resulting cells harvested and allowed to attach to laminin/poly-L-omithine-coated 96-well plates for 24 hrs. Cells were then imaged under transmitted light or to detect GFP fluorescence using an automated confocal microscope.

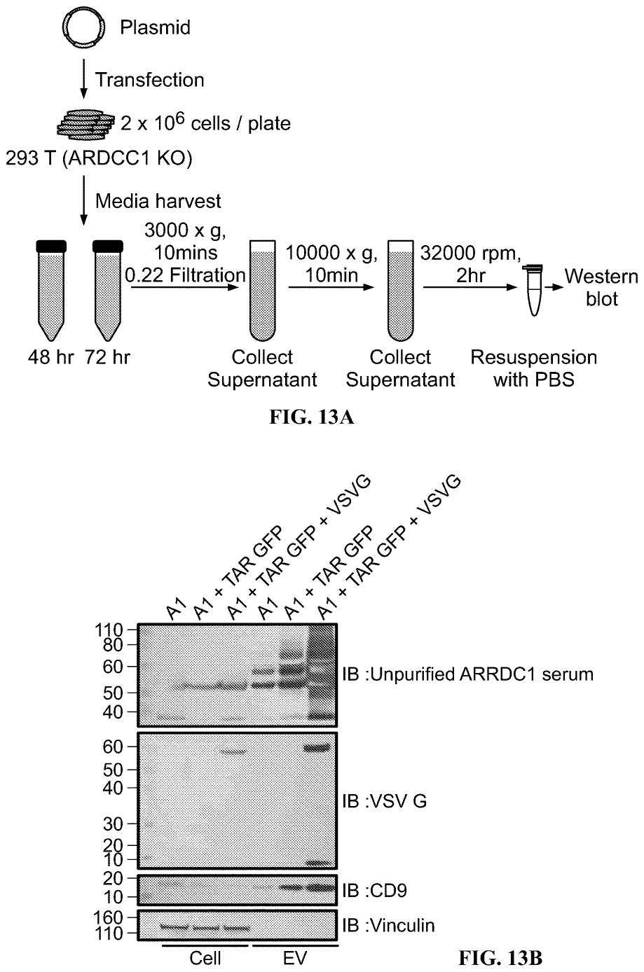

As shown in FIG. 14, with both 24- and 48-hour incubation with ARRDC1-GFP-VSVG extracellular vesicles, the delivery of the target protein (GFP) was detectable by fluorescence imaging in the majority of cells. Prolonged exposure up to 48 hours did not cause overt toxicity as measured by the ability to recover viable neurons that attached to to laminin/poly-L-ornithine-coated 96-well plates. Based upon the number of GFP-positive cells, these data indicate the ability of ARMM particles to deliver payload across more than just the outer cell layer. Overall, these results further demonstrate the effectiveness of the use of ARRDC1-mediated microvesicles for intracellular delivery of therapeutic macromolecules to cells in the human nervous system.

### Example 9: ARRDC1-Mediated Delivery of shRNA Payloads to Schwann Like Cells. Cell Culture and Transfection

Expi293F human suspension cells were cultured in Expi293® Expression Medium (Thermo Fisher Scientific, Waltham, MA). Cells were grown at 37° C. in 5% CO₂. Transfections in Expi293F™ cells were performed using ExpiFectamine® (Thermo Fisher Scientific). Before transfection, cell density and viability were checked, and the desired viability was >95%. The transfections were done when the density of cells was around 3×10⁶ cells/mL in small F-125 mL flasks. The ExpiFectamine® 293 Reagent bottle was gently inverted 4-5 times before use to ensure thorough mixing. Then 30 μL of ExpiFectamine® was diluted with 500 uL Opti-MEM® Reduced Serum Media (Thermo Fisher Scientific) then mixed by swirling or inversion. 10 μg total plasmid DNA was diluted in 500 μL Opti-MEM® Medium in another tube, then mixed by swirling or inversion. Tubes were incubated at room temperature for 5 minutes. After that the diluted ExpiFectamine® 293 Reagent was added to the diluted plasmid DNA and the mixture was mixed by swirling or inversion. The transfection mixture was incubated at room temperature for 15 minutes. After incubation the mixture was slowly transferred to the cells, swirling the culture flask gently during addition. Cells were then placed on the shaker in the incubator. 14-18 hours post-transfection, cell density and viability were checked, cells were spun down and resuspended into 40 mL fresh medium (around 1-2×10\hat{ }6 cells/mL). After an additional 48 hour culture, the conditioned medium was collected by initial centrifugation at 500 g for 10 min to remove the cells, followed by another round of centrifugation at 2000 g or 4000 rpm for 10 min. The collected conditioned medium was then filtered through 0.2 μm filter.

### ARMMs Purification

The conditioned medium was subjected to ultracentrifugation at 174,000×g for 2 hours. The medium was then aspirated, and the pellets enriched with ARMMs were resuspended in 170 μl ice-cold PBS.

### Nanoparticle Tracking Analysis (NTA)

ARMMs were analyzed and quantitated by the ZetaView® instrument (Particle Metrix GmbH, Inning am Ammersee, Germany). Samples containing vesicles were diluted with phosphate-buffered saline (PBS). The samples were subject to nanoparticle tracking analysis after dilution.

### Exosome Uptake Assay

Recipient human Schwann-like cells, sNF02.2, were seeded in 96- or 24-well plates and incubated with ARMMs as indicated in each experiment. After incubation, cells were washed with PBS and then treated with Gibco TrypL Express Enzyme (1×, Thermo Fisher Scientific) to harvest. Collected cells were then subjected to protein or RNA analysis.

### Quantitative RT-PCR

Aaa Total RNA was extracted from cells using miRNeasy Micro Kit (Qiagen, Germantown, MD) and subjected to treatment with DNase I (Invitrogen, Carlsbad, CA). First strand cDNA synthesis was conducted using SuperScript® IV VILO (Invitrogen). For quantitative PCR reaction, TaqMan© Gene Expression Master Mix (Thermo Fisher Scientific) was used per manufacturer's instructions. A QuantStudio© 6 Pro Real-Time PCR System (Applied Biosystems, Bedford, MA) was used for quantitative PCR analysis of cDNAs.

Digital PCR (ddPCR)

Total RNA was extracted from ARMMs (50 μl) using exoRNeasy Midi Kit (50) (Qiagen) and subjected to treatment with DNase I (Invitrogen). First strand cDNA synthesis was conducted using miRCURY LNA RT Kit (Qiagen). For the preparation of the ddPCR reaction mixture, NaicaC PCR MIX 5× (Stilla Technologies, Beverly, MA), Evagreen® Dye and qPCR Master Mixes, 20× in Water (Biotium, Hayward, CA) and custom designed miRCURY LNA miRNA PCR Assays (Qiagen) were used. Each ddPCR assay mixture (25 μl) was loaded into each inlet of Sapphire chip (Stilla Technologies). Naica® System instrument (Stilla Technologies) was used to perform the ddPCR analysis of shRNA content.

Use of TAR/Tat System for shRNA Payloading

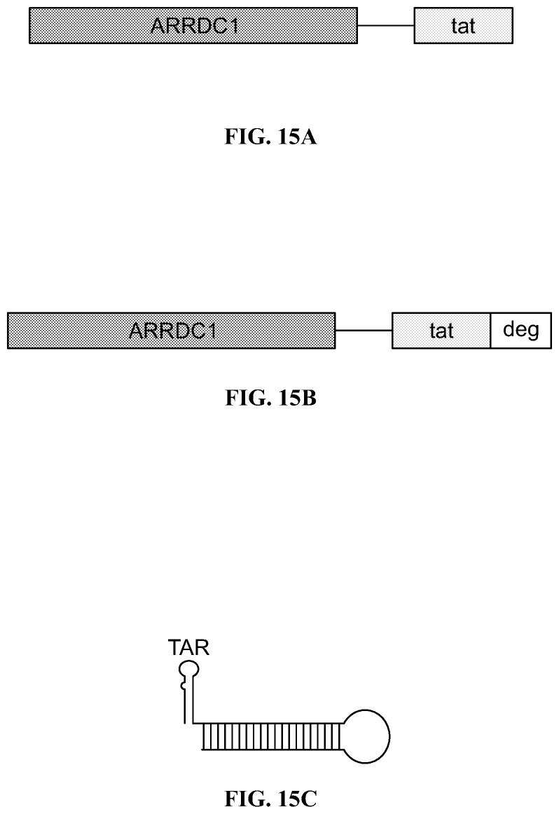

The TAR/tat system was used to package shRNAs into ARMMs. The transactivator of transcription (Tat) protein binds specifically to the stem-loop containing trans-activating response (TAR) element RNA. As shown in FIGS. 15A-15C, three expression constructs were created: 1) construct containing a Tat peptide (BIVtat65-81) fused via a linker to the C-terminus of ARRDC1 (FIG. 15A); 2) construct containing a Tat peptide (BIVtat65-81) fused via a linker to the C-terminus of ARRDC1 optionally further comprising a degron sequence; and 3) construct containing TAR was fused directly to the 5′ end of a cargo shRNA.

Packaging of shRNA into ARMMs

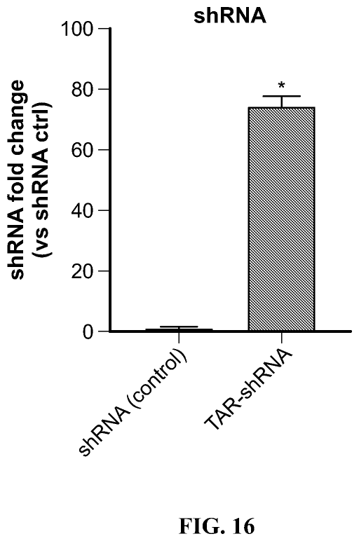

Packaging efficiency of shRNA targeting Pmp22 into ARMMs was evaluated by transfecting ARRDC1-Tat with control shRNA or TAR-shRNA into Expi293F production cells. ARMMs were harvested and subjected to shRNA analysis via ddPCR. Pmp22 targeting shRNAs were significantly more enriched in ARMMs of ARRDC1-Tat and TAR-shRNA co-transfection (FIG. 16) indicating that the Tat-TAR system successfully packaged TAR fused shRNAs into ARMMs.

ARMM-Mediated Functional Delivery of shRNA Payloads in Human Schwann Cell-Like sNF02.2

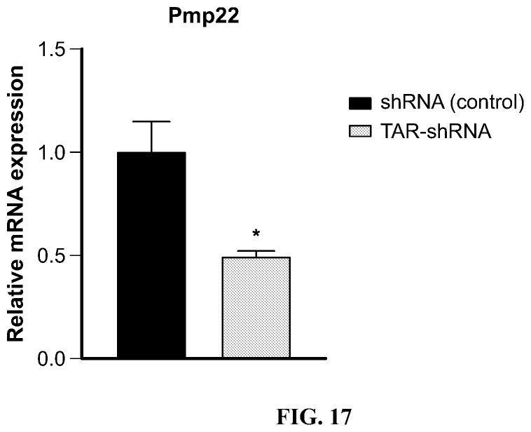

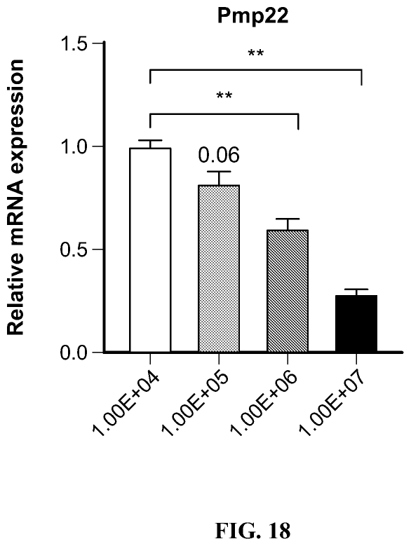

Payload delivery and success of shRNA mediated knockdown of Pmp22 target in sNF02.2 were further evaluated (FIGS. 17 and 18). Recipient cells (sNF02.2) were incubated with ARMMs containing either TAR-shRNA or control shRNA for 48 hrs. As shown, treatment of cells with ARMMs containing TAR-sRNAs led to ~50% reduction of Pmp22 mRNA levels compared to controls, suggesting successful delivery and functional activity of TAR-shRNA payloaded ARMMs in the recipient cells (FIG. 17). Furthermore, treatment of sNF02.2 cells with increasing amounts of ARMMs containing TAR-sRNAs led to increased reduction of Pmp22 mRNA levels, suggesting that uptake of ARMMs payloaded with shRNA is dose dependent in vitro (FIG. 18).

### Example 10: Design and Production of Genetic Constructs

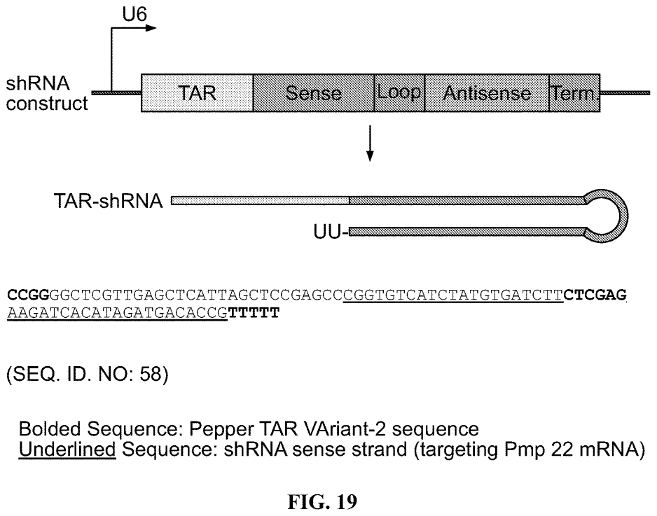

In this embodiment, an exemplary TAR-shRNA construct is provided; briefly, the DNA sequence of a TAR (Pepper TAR Variant-2, 28 nucleotides) was fused directly to the 5′ end of the shRNA sequence (human Pmp22 targeting sequence) in the pRP [Exp]-U6 plasmid. (FIG. 19).

Forward:
(SEQ ID NO: 58)
CCGGGGCTCGTTGAGCTCATTAGCTCCGAGCCCGGTGTCATCTATGTGATCTTCTCGAG
AAGATCACATAGATGACACCGTTTTT

The entire sequence of the exemplary construct is thusly provided.

(SEQ. ID. NO: 59)
CAACTTTGTATAGAAAAGTTGGAGGGCCTATTTCCCATGATTCCTTCATATTTGCATATACG
ATACAAGGCTGTTAGAGAGATAATTGGAATTAATTTGACTGTAAACACAAAGATATTAGTAC
AAAATACGTGACGTAGAAAGTAATAATTTCTTGGGTAGTTTGCAGTTTTAAAATTATGTTTT
AAAATGGACTATCATATGCTTACCGTAACTTGAAAGTATTTCGATTTCTTGGCTTTATATAT
CTTGTGGAAAGGACGAAACACCGGGGCTCGTTGAGCTCATTAGCTCCGAGCCCGGTGTCATC
TATGTGATCTTCTCGAGAAGATCACATAGATGACACCGTTTTTCAAGTTTGTACAAAAAAGC
AGGCTGGGTTGCGCCTTTTCCAAGGCAGCCCTGGGTTTGCGCAGGGACGCGGCTGCTCTGGG
CGTGGTTCCGGGAAACGCAGCGGCGCCGACCCTGGGTCTCGCACATTCTTCACGTCCGTTCG
CAGCGTCACCCGGATCTTCGCCGCTACCCTTGTGGGCCCCCCGGCGACGCTTCCTGCTCCGC
CCCTAAGTCGGGAAGGTTCCTTGCGGTTCGCGGCGTGCCGGACGTGACAAACGGAAGCCGCA
CGTCTCACTAGTACCCTCGCAGACGGACAGCGCCAGGGAGCAATGGCAGCGCGCCGACCGCG
ATGGGCTGTGGCCAATAGCGGCTGCTCAGCAGGGCGCGCCGAGAGCAGCGGCCGGGAAGGGG
CGGTGCGGGAGGCGGGGTGTGGGGCGGTAGTGTGGGCCCTGTTCCTGCCCGCGCGGTGTTCC
GCATTCTGCAAGCCTCCGGAGCGCACGTCGGCAGTCGGCTCCCTCGTTGACCGAATCACCGA
CCTCTCTCCCCAGGACCCAGCTTTCTTGTACAAAGTGGGCCACCATGGTGAGCAAGGGCGAG
GAGCTGTTCACCGGGGTGGTGCCCATCCTGGTCGAGCTGGACGGCGACGTAAACGGCCACAA
GTTCAGCGTGTCCGGCGAGGGCGAGGGCGATGCCACCTACGGCAAGCTGACCCTGAAGTTCA
TCTGCACCACCGGCAAGCTGCCCGTGCCCTGGCCCACCCTCGTGACCACCCTGACCTACGGC
GTGCAGTGCTTCAGCCGCTACCCCGACCACATGAAGCAGCACGACTTCTTCAAGTCCGCCAT
GCCCGAAGGCTACGTCCAGGAGCGCACCATCTTCTTCAAGGACGACGGCAACTACAAGACCC
GCGCCGAGGTGAAGTTCGAGGGCGACACCCTGGTGAACCGCATCGAGCTGAAGGGCATCGAC
TTCAAGGAGGACGGCAACATCCTGGGGCACAAGCTGGAGTACAACTACAACAGCCACAACGT
CTATATCATGGCCGACAAGCAGAAGAACGGCATCAAGGTGAACTTCAAGATCCGCCACAACA
TCGAGGACGGCAGCGTGCAGCTCGCCGACCACTACCAGCAGAACACCCCCATCGGCGACGGC
CCCGTGCTGCTGCCCGACAACCACTACCTGAGCACCCAGTCCGCCCTGAGCAAAGACCCCAA
CGAGAAGCGCGATCACATGGTCCTGCTGGAGTTCGTGACCGCCGCCGGGATCACTCTCGGCA
TGGACGAGCTGTACAAGTAACAACTTTATTATACATAGTTGATGGCCGGCCGCTTCGAGCAG
ACATGATAAGATACATTGATGAGTTTGGACAAACCACAACTAGAATGCAGTGAAAAAAATGC
TTTATTTGTGAAATTTGTGATGCTATTGCTTTATTTGTAACCATTATAAGCTGCAATAAACA
AGTTAACAACAACAATTGCATTCATTTTATGTTTCAGGTTCAGGGGGAGGTGTGGGAGGTTT
TTTAAAGCAAGTAAAACCTCTACAAATGTGGTAGCGGCCGCGGCGCTCTTCCGCTTCCTCGC
TCACTGACTCGCTGCGCTCGGTCGTTCGGCTGCGGCGAGCGGTATCAGCTCACTCAAAGGCG
GTAATACGGTTATCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGACAGGACTATAA
AGATACCAGGCGTTTCCCCCTGGAAGCTCCCTCGTGCGCTCTCCTGTTCCGACCCTGCCGCT
TACCGGATACCTGTCCGCCTTTCTCTCTTCGGGAAGCGTGGCGCTTTCTCATAGCTCACGCT
GTAGGTATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTAAGACA
CGACTTATCGCCACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGAACAGTATTTGGT
ATCTGCGCTCTGCTGAAGCCAGTTACCTTCGGAAAAAGAGTTGGTAGCTCTTGATCCGGCAA
ACAAACCACCGCTGGTAGCGGTGGTTTTTTTGTTTGCAAGCAGCAGATTACGCGCAGAAAAA
AAGGATCTCAAGAAGATCCTTTGATCTTTTCTACGGGGTCTGACGCTCAGTGGAACGAAAAC
TCACGTTAAGGGATTTTGGTCATGAGATTATCAAAAAGGATCTTCACCTAGATCCTTTTAAA
TTAAAAATGAAGTTTTAAATCAATCTAAAGTATATATGAGTAAACTTGGTCTGACAGTTACC
AATGCTTAATCAGTGAGGCACCTATCTCAGCGATCTGTCTATTTCGTTCATCCATAGTTGCC
TGACTCCCCGTCGTGTAGATAACTACGATACGGGAGGGCTTACCATCTGGCCCCAGTGCTGC
AATGATACCGCGAGACCCACGCTCACCGGCTCCAGATTTATCAGCAATAAACCAGCCAGCCG
GAAGGGCCGAGCGCAGAAGTGGTCCTGCAACTTTATCCGCCTCCATCCAGTCTATTAATTGT
TGCCGGGAAGCTAGAGTAAGTAGTTCGCCAGTTAATAGTTTGCGCAACGTTGTTGCCATTGC
TACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCA
TAATTCTCTTACTGTCATGCCATCCGTAAGATGCTTTTCTGTGACTGGTGAGTACTCAACCA
AGTCATTCTGAGAATAGTGTATGCGGCGACCGAGTTGCTCTTGCCCGGCGTCAATACGGGAT
AATACCGCGCCACATAGCAGAACTTTAAAAGTGCTCATCATTGGAAAACGTTCTTCGGGGCG
AAAACTCTCAAGGATCTTACCGCTGTTGAGATCCAGTTCGATGTAACCCACTCGTGCACCCA
ACTGATCTTCAGCATCTTTTACTTTCACCAGCGTTTCTGGGTGAGCAAAAACAGGAAGGCAA
AATGCCGCAAAAAAGGGAATAAGGGCGACACGGAAATGTTGAATACTCATACTCTTCCTTTT
TCAATATTATTGAAGCATTTATCAGGGTTATTGTCTCATGAGCGGATACATATTTGAATGTA
TTTAGAAAAATAAACAAATAGGGGTTCCGCGCACATTTCCCCGAAAAGTGCCACCTGACGTC
TAAGAAACCATTATTATCATGACATTAACCTATAAAAATAGGCGTATCACGAGGCCCTTTCG
TCGGCGCGCCGCGGCCGC

In this additional embodiment, a second exemplary construct is provided; briefly, a DNA sequence of Tat (57 bp) was inserted at the C terminus of ARRDC1. A short peptide linker was placed between the C-terminus of ARRDC1 and Biv-Tat peptide (65-81). Additionally, in this exemplary construct, a pRP[Exp]-Neo-CMV vector backbone was utilized.

Sequence of Biv-Tat:
(SEQ. ID. NO: 60)
AGCGGCCCGCGCCCGCGCGGCACCCGCGGCAAAGGCCGCCGCATTCGCCGCCGCGGC
Sequence for Short Peptide Linker:
(SEQ. ID. NO: 61)
GGCGGCAGCAGCGGC
The entire sequence of the exemplary construct is thusly provided.
(SEQ. ID. NO: 62)
CAACTTTGTATAGAAAAGTTGTAGTTATTAATAGTAATCAATTACGGGGTCATTAGTTCATA
GCCCATATATGGAGTTCCGCGTTACATAACTTACGGTAAATGGCCCGCCTGGCTGACCGCCC
AACGACCCCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTACATCAAG
TGTATCATATGCCAAGTACGCCCCCTATTGACGTCAATGACGGTAAATGGCCCGCCTGGCAT
TATGCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTGACT
CACGGGGATTTCCAAGTCTCCACCCCATTGACGTCAATGGGAGTTTGTTTTGGCACCAAAAT
CAACGGGACTTTCCAAAATGTCGTAACAACTCCGCCCCATTGACGCAAATGGGCGGTAGGCG
TGTACGGTGGGAGGTCTATATAAGCAGAGCTGGTTTAGTGAACCGTCAGATCCAAGTTTGTA
CAAAAAAGCAGGCTGCCACCATGGGGCGAGTGCAGCTCTTCGAGATCAGCCTGAGCCACGGC
CGCGTCGTCTACAGCCCCGGGGAGCCGTTGGCTGGGACCGTGCGCGTGCGCCTGGGGGCACC
GCTGCCGTTCCGAGCCATCCGGGTGACCTGCATAGGTTCCTGCGGGGTCTCCAACAAGGCTA
ATGACACAGCGTGGGTAGTGGAGGAGGGTTACTTCAACAGTTCCCTGTCGCTGGCAGACAAG
GGGAGCCTGCCCGCTGGAGAGCACAGCTTCCCCTTCCAGTTCCTGCTTCCTGCCACTGCACC
CACGTCCTTTGAGGGTCCTTTCGGGAAGATCGTGCACCAGGTGAGGGCCGCCATCCACACGC
CACGGTTTTCCAAGGATCACAAGTGCAGCCTCGTGTTCTATATCTTGAGCCCCTTGAACCTG
AACAGCATCCCAGACATTGAGCAACCCAACGTGGCCTCTGCCACCAAGAAGTTCTCCTACAA
GCTGGTGAAGACGGGCAGCGTGGTCCTCACAGCCAGCACTGATCTCCGCGGCTATGTGGTGG
GGCAGGCACTGCAGCTGCATGCCGACGTTGAGAACCAGTCAGGCAAGGACACCAGCCCTGTG
GTGGCCAGTCTGCTGCAGAAAGTGTCCTATAAGGCCAAGCGCTGGATCCACGACGTACGGAC
CATTGCGGAGGTGGAGGGTGCGGGCGTCAAGGCCTGGCGGCGGGCGCAGTGGCACGAGCAGA
TCCTGGTGCCTGCCTTGCCCCAGTCGGCCCTGCCGGGCTGCAGCCTCATCCACATCGACTAC
TACTTACAGGTCTCTCTGAAGGCGCCGGAAGCTACTGTGACCCTCCCGGTCTTCATTGGCAA
TATTGCTGTGAACCATGCCCCAGTGAGCCCCCGGCCAGGCCTGGGGCTGCCTCCTGGGGCCC
CACCCCTGGTGGTGCCTTCCGCACCACCCCAGGAGGAGGCTGAGGCTGAGGCTGCGGCTGGC
GGCCCCCACTTCTTGGACCCCGTCTTCCTCTCCACCAAGAGCCATTCGCAGCGGCAGCCCCT
GCTGGCCACCTTGAGTTCTGTGCCTGGTGCGCCGGAGCCCTGCCCTCAGGATGGCAGCCCTG
CCTCACACCCGCTGCACCCTCCCTTGTGCATTTCAACAGGTGCCACTGTCCCCTACTTTGCA
GAGGGCTCCGGGGGGCCAGTGCCCACTACCAGCACCTTGATTCTTCCTCCAGAGTACAGTTC
TTGGGGCTACCCCTATGAGGCCCCACCGTCTTATGAGCAGAGCTGCGGCGGCGTGGAACCCA
GCCTGACCCCTGAGAGCGGCGGCAGCAGCGGCAGCGGCCCGCGCCCGCGCGGCACCCGCGGC
AAAGGCCGCCGCATTCGCCGCCGCGGCTAAACCCAGCTTTCTTGTACAAAGTGGTGATGGCC
GGCCGCTTCGAGCAGACATGATAAGATACATTGATGAGTTTGGACAAACCACAACTAGAATG
CAGTGAAAAAAATGCTTTATTTGTGAAATTTGTGATGCTATTGCTTTATTTGTAACCATTAT
AAGCTGCAATAAACAAGTTAACAACAACAATTGCATTCATTTTATGTTTCAGGTTCAGGGGG
AGGTGTGGGAGGTTTTTTAAAGCAAGTAAAACCTCTACAAATGTGGTACGCGTTGACATTGA
TTATTGACTAGTTATTAATAGTAATCAATTACGGGGTCATTAGTTCATAGCCCATATATGGA
GTTCCGCGTTACATAACTTACGGTAAATGGCCCGCCTGGCTGACCGCCCAACGACCCCCGCC
CATTGACGTCAATAATGACGTATGTTCCCATAGTAACGCCAATAGGGACTTTCCATTGACGT
CAATGGGTGGAGTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACA
TGACCTTATGGGACTTTCCTACTTGGCAGTACATCTACGTATTAGTCATCGCTATTACCATG
GTGATGCGGTTTTGGCAGTACATCAATGGGCGTGGATAGCGGTTTGACTCACGGGGATTTCC
AAGTCTCCACCCCATTGACGTCAATGGGAGTTTGTTTTGGCACCAAAATCAACGGGACTTTC
CAAAATGTCGTAACAACTCCGCCCCATTGACGCAAATGGGCGGTAGGCGTGTACGGTGGGAG
GTCTATATAAGCAGAGCTCTCTGGCTAACTAGAGAACCCACTGCGCCACCATGATTGAACAA
GATGGATTGCACGCAGGTTCTCCGGCCGCTTGGGTGGAGAGGCTATTCGGCTATGACTGGGC
ACAACAGACAATCGGCTGCTCTGATGCCGCCGTGTTCCGGCTGTCAGCGCAGGGGCGCCCGG
TTCTTTTTGTCAAGACCGACCTGTCCGGTGCCCTGAATGAACTGCAAGACGAGGCAGCGCGG
CTATCGTGGCTGGCCACGACGGGCGTTCCTTGCGCAGCTGTGCTCGACGTTGTCACTGAAGC
GGGAAGGGACTGGCTGCTATTGGGCGAAGTGCCGGGGCAGGATCTCCTGTCATCTCACCTTG
CTCCTGCCGAGAAAGTATCCATCATGGCTGATGCAATGCGGCGGCTGCATACGCTTGATCCG
GCTACCTGCCCATTCGACCACCAAGCGAAACATCGCATCGAGCGAGCACGTACTCGGATGGA
AGCCGGTCTTGTCGATCAGGATGATCTGGACGAAGAGCATCAGGGGCTCGCGCCAGCCGAAC
TGTTCGCCAGGCTCAAGGCGAGCATGCCCGACGGCGAGGATCTCGTCGTGACCCATGGCGAT
GCCTGCTTGCCGAATATCATGGTGGAAAATGGCCGCTTTTCTGGATTCATCGACTGTGGCCG
GCTGGGTGTGGCGGACCGCTATCAGGACATAGCGTTGGCTACCCGTGATATTGCTGAAGAGC
TTGGCGGCGAATGGGCTGACCGCTTCCTCGTGCTTTACGGTATCGCCGCTCCCGATTCGCAG
CGCATCGCCTTCTATCGCCTTCTTGACGAGTTCTTCTGACTCGAGTCTAGAGGGCCCGTTTA
AACCCGCTGATCAGCCTCGACTGTGCCTTCTAGTTGCCAGCCATCTGTTGTTTGCCCCTCCC
CCGTGCCTTCCTTGACCCTGGAAGGTGCCACTCCCACTGTCCTTTCCTAATAAAATGAGGAA
ATTGCATCGCATTGTCTGAGTAGGTGTCATTCTATTCTGGGGGGGGGGTGGGGCAGGACAG
CAAGGGGGAGGATTGGGAAGACAATAGCAGGCATGCTGGGGATGCGGTGGGCTCTATGGGCG
GCCGCGGCGCTCTTCCGCTTCCTCGCTCACTGACTCGCTGCGCTCGGTCGTTCGGCTGCGGC
GAGCGGTATCAGCTCACTCAAAGGCGGTAATACGGTTATCCACAGAATCAGGGGATAACGCA
GGAAAGAACATGTGAGCAAAAGGCCAGCAAAAGGCCAGGAACCGTAAAAAGGCCGCGTTGCT
GGCGTTTTTCCATAGGCTCCGCCCCCCTGACGAGCATCACAAAAATCGACGCTCAAGTCAGA
GGTGGCGAAACCCGACAGGACTATAAAGATACCAGGCGTTTCCCCCTGGAAGCTCCCTCGTG
CGCTCTCCTGTTCCGACCCTGCCGCTTACCGGATACCTGTCCGCCTTTCTCTCTTCGGGAAG
CGTGGCGCTTTCTCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGTAACAG
GATTAGCAGAGCGAGGTATGTAGGCGGTGCTACAGAGTTCTTGAAGTGGTGGCCTAACTACG
GCTACACTAGAAGAACAGTATTTGGTATCTGCGCTCTGCTGAAGCCAGTTACCTTCGGAAAA
AGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTATCAAAA
AGGATCTTCACCTAGATCCTTTTAAATTAAAAATGAAGTTTTAAATCAATCTAAAGTATATA
TGAGTAAACTTGGTCTGACAGTTACCAATGCTTAATCAGTGAGGCACCTATCTCAGCGATCT
GTCTATTTCGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACCGGCTCCAGA
TTTATCAGCAATAAACCAGCCAGCCGGAAGGGCCGAGCGCAGAAGTGGTCCTGCAACTTTAT
CCGCCTCCATCCAGTCTATTAATTGTTGCCGGGAAGCTAGAGTAAGTAGTTCGCCAGTTAAT
AGTTTGCGCAACGTTGTTGCCATTGCTACAGGCATCGTGGTGTCACGCTCGTCGTTTGGTAT
GGCTTCATTCAGCTCCGGTTCCCAACGATCAAGGCGAGTTACATGATCCCCCATGTTGTGCA
AAAAAGCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGATGCTT
TTCTGTGACTGGTGAGTACTCAACCAAGTCATTCTGAGAATAGTGTATGCGGCGACCGAGTT
GCTCTTGCCCGGCGTCAATACGGGATAATACCGCGCCACATAGCAGAACTTTAAAAGTGCTC
ATCATTGGAAAACGTTCTTCGGGGCGAAAACTCTCAAGGATCTTACCGCTGTTGAGATCCAG
TTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAA
TGTTGAATACTCATACTCTTCCTTTTTCAATATTATTGAAGCATTTATCAGGGTTATTGTCT
CATGAGCGGATACATATTTGAATGTATTTAGAAAAATAAACAAATAGGGGTTCCGCGCACAT
TTCCCCGAAAAGTGCCACCTGACGTCTAAGAAACCATTATTATCATGACATTAACCTATAAA
AATAGGCGTATCACGAGGCCCTTTCGTCGGCGCGCCGCGGCCGC

### Example 10: High Efficiency Editing Delivered by ARMMs (ABE8-PMP22TA TAg)

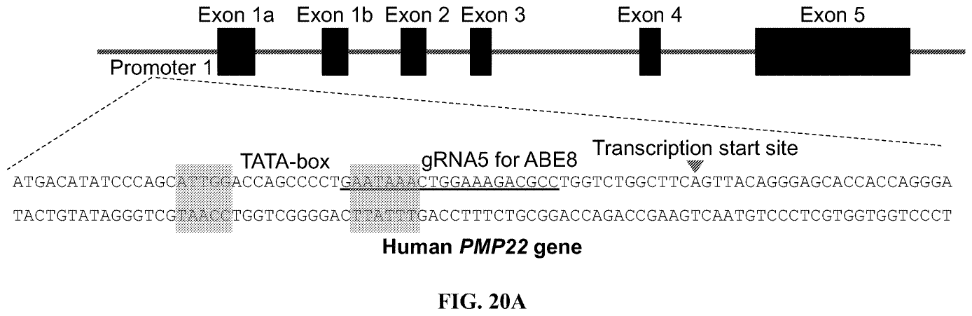

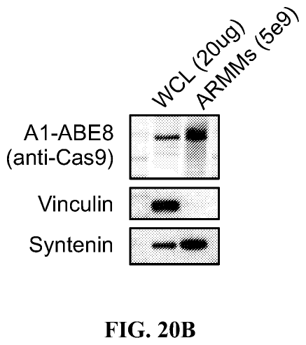

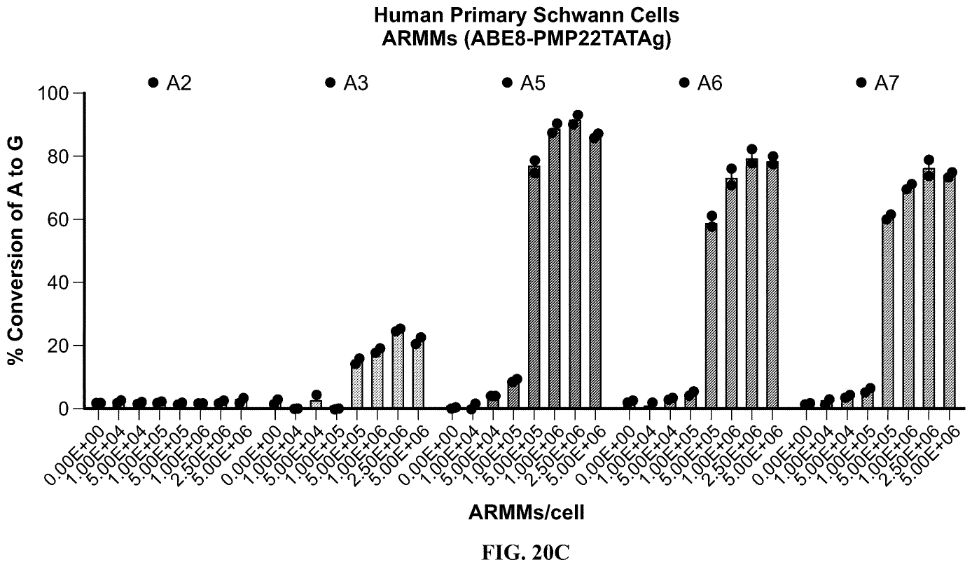

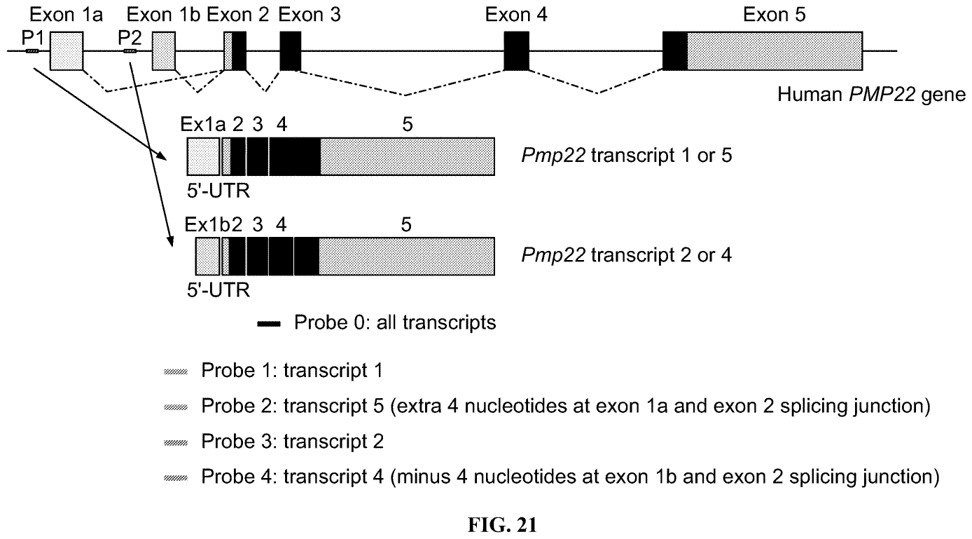

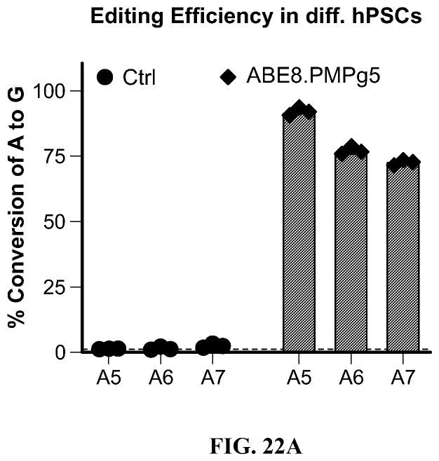

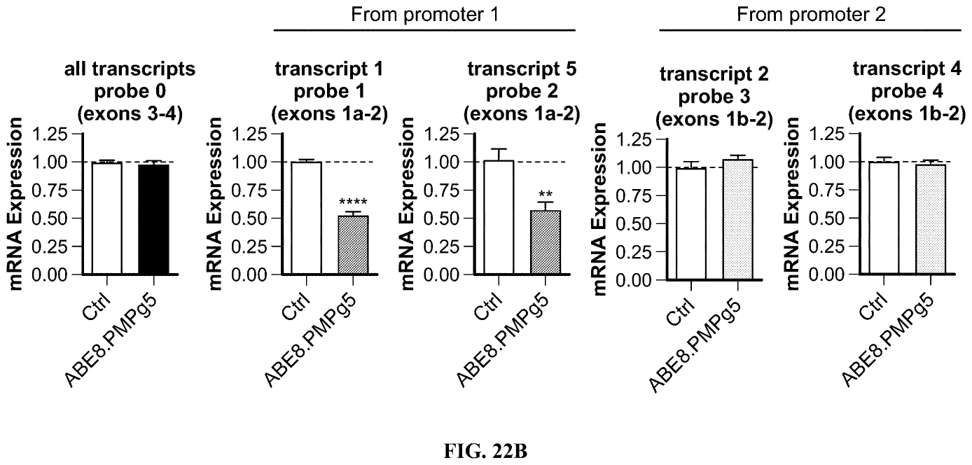

ARMMs carrying a base editor, in this embodiment, ABE8, and suitable gRNA (as depicted in FIG. 20A) were designed to be delivered to human primary schwann cells for editing of the human PMP22 gene. The human PMP222 gene comprises various promoters and transcripts as generally depicted in FIG. 21. As shown in FIG. 22A and FIG. 22B, Pmp22 gene expression from promoter 1 in differentiating hPSCs with a base-edited TATA box was decreased when these cells were treated with ARMMs.

FIG. 20A shows the structure of the human PMP22 gene with black bars depicting exons as shown. The expanded view shows the sequence for promoter 1 (SEQ. ID. NO: 63), with the TATA box highlighted in pink and the protospacer for gRNA5 is shown by underlining.

### Western Blot Analysis

1×10⁹ to 1×10¹⁰ ARMMs were lysed in Invitrogen Novex LDS Loading Buffer (Thermo Scientific, B0007) with β-mercaptoethanol included (5% v/v), and heated at 95° C. for 10 minutes. The proteins were separated on a Bolt 4-12% acrylamide Bis-Tris gel (Thermo Scientific NW04125BOX) and transferred to a nitrocellulose membrane using the Trans-Blot Turbo system (Bio-Rad). The membrane was blocked with 5% milk in TBS-T for 1 hour at room temperature, and probed with primary antibodies to Cas9 (Cell Signaling Technology 14697S, 1:1000), Syntenin (Abcam, ab133267, 1:1000), and vinculin (CST, 13901S, 1:1000) overnight at 4° C. with gentle rocking. The membrane was then washed 3 times for 5 minutes each in TBS-T and probed with a HRP-conjugated secondary antibody to mouse or rabbit IgG as appropriate (Cell Signaling Technology, 7076S or 7074S), at a dilution of 1:2000 for 2 h with gentle rocking at room temperature. The membranes were then washed 4 times for 5 minutes in TBS-T, and developed using Pierce Western Blotting Substrate (Thermo Scientific, 32106) with imaging on an iBright imaging system (Thermo Scientific). When required, we stripped the membranes using Restore Western Blot stripping buffer (Fisher Scientific PI46430). The results of these experiments are shown in FIG. 20B.

Evaluation of gRNA in ARMMs

To quantify gRNA packaged in ARMMs, a custom TaqMan probe was ordered from ThermoFisher Scientific (Assay ID: AP7DWZ9). This probe was designed against the scaffold sequence in the single gRNA (sgRNA). The quantification of gRNA included three steps: preparation of RNA samples, cDNA synthesis, and qPCR. RNA samples were prepared using TaqMan™ Gene Expression Cells-to-CT™ Kit (ThermoFisher Scientific, AM1728). 1 μL of ARMMs preparation was added to 21.5 uL of cell lysis buffer with DNase I (100×), of which the mixture was incubated at RT for 5 min before 2.5 uL of STOP solution was added, resulting in 25× dilution of the original ARMMs preparation. Reverse transcription (RT) was performed with the 20×RT enzyme mix and 2×RT buffer from the kit in a 25 uL reaction with 1 μL of prepared RNA samples. qPCR was set up as described above using the Taqman probe customized for gRNA. A serial dilution of synthetic gRNA with known concentration was included in the RT and qPCR, and used to create a standard curve to interpolate the concentrations of test samples. The results of these experiments are summarized in the following table.

TABLE 2
Payloading of ABE8 and gRNAs
in ARMMs (ABE8-PMP22TATAg)

| Payload in ARMMs | ABE8 copies/EV | gRNA copies/EV |
|---|---|---|
| ABE8 + PMP22TATAg5 | 2.2 | 0.73 |
| ABE8 + PMP22TATAg5 | 11.1 | 2.52 |
| ABE8 + PMP22TATAg5 | 10.3 | 5.01 |

### ARMMs Uptake and CRISPR/Cas9 Genome Editing Evaluation

Cells were seeded in 96-well plates at the densities of 5000 cells/well for cell lines or 10,000 cells/well for mouse primary cells. After 3 hours, ARMMs were applied to cells at the indicated concentrations (particles/cell). Cells treated for 96 hours were collected for genomic DNA (gDNA) extraction or stored at −80 C for later processing. gDNA was extracted using the DNeasv 96 Blood & Tissue Kit (Qiagen, 69581) and following the manufacturer's instructions. Targeted PCR was performed to amplify the genomic regions harboring the genome editing sites. 5 μL of gDNA solution was used as template in the PCR reaction using Q5 High-Fidelity 2× Master Mix (NEB, M0492) with the manufacturer's protocols. PCR products were confirmed on E-gels and purified using the QIAquick 96 PCR Purification Kit (Qiagen, 28181). 1-5 uL of the eluted PCR products were submitted for Sanger sequencing to have an initial evaluation of editing before the rest of PCR products were submitted for Amplicon deep sequencing. The results of these experiments are shown in FIG. 20C and FIG. 22A.

### Quantitative PCR

Cells treated with ARMMs in 96-well plates were collected and stored at −80 C. RNA samples were prepared using TaqMan™ Gene Expression Cells-to-CT™ Kit (ThermoFisher Scientific, AM1728). Briefly, 50 μL of cell lysis buffer with DNase I (100×) was dispended to each well with pipette tips scraping well bottom to dislodge cells followed by incubation at room temperature (RT) for 5 min. 5 uL STOP solution from the kit was dispensed to each well to stop the lysis. The High-Capacity cDNA Reverse Transcription Kit (ThermoFisher Scientific, 43-749-66) was used for cDNA synthesis with 13 μL of the above-prepared RNA samples as template in a 20 uL reaction. At the end of the reaction, cDNA samples were diluted 4-fold. For qPCR reaction in 384-well plate, 10 uL reaction was assembled with 5 μL of TaqMan 2× Gene Expression Master Mix (Thermo Fisher Scientific, 4369016), 0.5 μL of 20× TaqMan primer probe for mouse Hprt gene (VIC), 0.5 μL of 20× TaqMan primer probe for the gene of interest (FAM), and 4 μL of prepared diluted cDNA. qPCR was run on QuantStudio Pro (ThermoFisher Scientific) and Cq values were used to evaluate gene expression levels normalized to the internal control Hprt expression. The results of these experiments are shown in FIG. 22B.

### REFERENCES

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

### EQUIVALENTS AND SCOPE

Those skilled in the art will recognize or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus, for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all the embodiments in which one or more elements, features, purposes, or aspects is excluded are not set forth explicitly herein.

What is claimed is:

1. An arrestin domain-containing protein 1 (ARRDC1)-mediated microvesicle (ARMM), comprising:
(i) a lipid bilayer and an ARRDC1 protein,
(ii) a molecule, and
(iii) a viral envelope protein.

2. The microvesicle of claim 1, wherein said viral envelope protein is vesicular stomatitis virus G (VSV-G).

3. The microvesicle of claim 1, wherein said viral envelope protein is rabies virus glycoprotein (RVG).

4. A microvesicle-producing cell comprising:
a recombinant expression construct encoding an ARRDC1 protein or a variant thereof under the control of a heterologous promoter,
and a viral envelope protein.

5. The microvesicle-producing cell of claim 4, wherein said viral envelope protein is VSV-G.

6. The microvesicle-producing cell of claim 4, wherein said viral envelope protein is RVG.

7. A method of delivering a molecule to a target cell, said method comprising contacting the target cell with the microvesicle of any of claims 1-3.

8. The method of claim 7, wherein said target cell is a cell of the peripheral nervous system (PNS).

9. The method of claim 8, wherein said cell of the peripheral nervous system is a Schwann cell.

10. A method of treating a disorder in a patient, the method consisting of administering to the patient a microvesicle of any of claims 1-3.

11. A method of treating a disorder in a patient, the method consisting of administering to the patient a microvesicle-producing cell of any of claims 4-6.

12. The method of claim 10 or 11, wherein said disorder is a disorder of the PNS.

13. The method of claim 12, wherein said disorder effects the myelin sheath of a PNS cell.

14. The method of claim 13, wherein said myelin sheath comprises a part of a Schwann cell.

15. The method of claim 10, wherein said disorder is a CMT disorder.

16. The method of claim 15, wherein said CMT disorder comprises CMT1A.

17. The method of claim 11, wherein said disorder is a CMT disorder.

18. The method of claim 15, wherein said CMT disorder comprises CMT1A.

19. The method of claim 10 or 11, wherein said disorder is repeat of a gene.

20. The method of claim 19, wherein said gene is PMP22.

21. The method of claim 10 or 11, wherein said disorder is schwannomatosis.

22. An arrestin domain-containing protein 1 (ARRDC1)-mediated microvesicle (ARMM), comprising:
(i) a lipid bilayer and an ARRDC1 protein, and
(ii) a therapeutic agent.

23. The microvesicle of claim 22, wherein said therapeutic agent is a nucleic acid molecule.

24. The microvesicle of claim 23, wherein said nucleic acid molecule consists of RNA.

25. The microvesicle of claim 24, wherein said RNA comprises siRNA, shRNA, or mRNA.

26. The microvesicle of claim 23, wherein said RNA comprises shRNA.

27. The method of claim 10 or 11, wherein said patient is a mammal.

28. The method of claim 27, wherein said mammal is a primate.

29. The method of claim 28, wherein said primate is a human.

30. The method of claim 29, wherein said human has a disorder of the PNS.

31. The method of claim 30, wherein said disorder of the PNS is a Schwann cell disorder.

32. The method of claim 31, wherein said Schwann cell disorder is selected from the group comprising CMT and schwannomatosis.

33. An arrestin domain-containing protein 1 (ARRDC1)-mediated microvesicle (ARMM) for use a medicament.

34. An arrestin domain-containing protein 1 (ARRDC1)-mediated microvesicle (ARMM) for use in the treatment of diseases of the PNS.

35. The use of said arrestin domain-containing protein 1 (ARRDC1)-mediated microvesicle (ARMM) of claim 34, wherein said diseases are CMT1A or schwannomas.

36. A composition substantially as shown and described.

37. A method substantially as shown and described.


SEQUENCE LISTING (63 sequences)

SEQ ID NO: 1 - protein, 4 aa, synthetic construct
PSAP

SEQ ID NO: 2 - skipped

SEQ ID NO: 3 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 4 - protein, 922 aa, Homo sapiens
MATASPRSDTSNNHSGRLQLQVTVSSAKLKRKKNWFGTAIYTEVVVDGEITKTAKSSSSSNPKWDEQLTVNVTPQTTLEFQVWSHRTLKADALLGKATIDLKQALLIHNRKLERVKEQLKLSLENKNGIAQTGELTVVLDGLVIEQENITNCSSSPTIEIQENGDALHENGEPSARTTARLAVEGTNGIDNHVPTSTLVQNSCCSYVVNGDNTPSSPSQVAARPKNTPAPKPLASEPADDTVNGESSSFAPTDNASVTGTPVVSEENALSPNCTSTTVEDPPVQEILTSSENNECIPSTSAELESEARSILEPDTSNSRSSSAFEAAKSRQPDGCMDPVRQQSGNANTETLPSGWEQRKDPHGRTYYVDHNTRTTTWERPQPLPPGWERRVDDRRRVYYVDHNTRTTTWQRPTMESVRNFEQWQSQRNQLQGAMQQFNQRYLYSASMLAAENDPYGPLPPGWEKRVDSTDRVYFVNHNTKTTQWEDPRTQGLQNEEPLPEGWEIRYTREGVRYFVDHNTRTTTFKDPRNGKSSVTKGGPQIAYERGFRWKLAHFRYLCQSNALPSHVKINVSRQTLFEDSFQQIMALKPYDLRRRLYVIFRGEEGLDYGGLAREWFFLLSHEVLNPMYCLFEYAGKNNYCLQINPASTINPDHLSYFCFIGRFIAMALFHGKFIDTGFSLPFYKRMLSKKLTIKDLESIDTEFYNSLIWIRDNNIEECGLEMYFSVDMEILGKVTSHDLKLGGSNILVTEENKDEYIGLMTEWRFSRGVQEQTKAFLDGFNEVVPLQWLQYFDEKELEVMLCGMQEVDLADWQRNTVYRHYTRNSKQIIWFWQFVKETDNEVRMRLLQFVTGTCRLPLGGFAELMGSNGPQKFCIEKVGKDTWLPRSHTCFNRLDLPPYKSYEQLKEKLLFAIEETEGFGQE

SEQ ID NO: 5 - protein, 34 aa, Homo sapiens
ETLPSGWEQRKDPHGRTYYVDHNTRTTTWERPQP

SEQ ID NO: 6 - protein, 34 aa, Homo sapiens
QPLPPGWERRVDDRRRVYYVDHNTRTTTWQRPTM

SEQ ID NO: 7 - protein, 39 aa, Homo sapiens
ENDPYGPLPPGWEKRVDSTDRVYFVNHNTKTTQWEDPRT

SEQ ID NO: 8 - protein, 34 aa, Homo sapiens
EPLPEGWEIRYTREGVRYFVDHNTRTTTFKDPRN

SEQ ID NO: 9 - protein, 870 aa, Homo sapiens
MASASSSRAGVALPFEKSQLTLKVVSAKPKVHNRQPRINSYVEVAVDGLPSETKKTGKRIGSSELLWNEIIILNVTAQSHLDLKVWSCHTLRNELLGTASVNLSNVLKNNGGKMENMQLTLNLQTENKGSVVSGGELTIFLDGPTVDLGNVPNGSALTDGSQLPSRDSSGTAVAPENRHQPPSTNCFGGRSRTHRHSGASARTTPATGEQSPGARSRHRQPVKNSGHSGLANGTVNDEPTTATDPEEPSVVGVTSPPAAPLSVTPNPNTTSLPAPATPAEGEEPSTSGTQQLPAAAQAPDALPAGWEQRELPNGRVYYVDHNTKTTTWERPLPPGWEKRTDPRGRFYYVDHNTRTTTWQRPTAEYVRNYEQWQSQRNQLQGAMQHFSQRFLYQSSSASTDHDPLGPLPPGWEKRQDNGRVYYVNHNTRTTQWEDPRTQGMIQEPALPPGWEMKYTSEGVRYFVDHNTRTTTFKDPRPGFESGTKQGSPGAYDRSFRWKYHQFRFLCHSNALPSHVKISVSRQTLFEDSFQQIMNMKPYDLRRRLYIIMRGEEGLDYGGIAREWFFLLSHEVLNPMYCLFEYAGKNNYCLQINPASSINPDHLTYFRFIGRFIAMALYHGKFIDTGFTLPFYKRMLNKRPTLKDLESIDPEFYNSIVWIKENNLEECGLELYFIQDMEILGKVTTHELKEGGESIRVTEENKEEYIMLLTDWRFTRGVEEQTKAFLDGFNEVAPLEWLRYFDEKELELMLCGMQEIDMSDWQKSTIYRHYTKNSKQIQWFWQVVKEMDNEKRIRLLQFVTGTCRLPVGGFAELIGSNGPQKFCIDKVGKETWLPRSHTCFNRLDLPPYKSYEQLREKLLYAIEETEGFGQE

SEQ ID NO: 10 - protein, 34 aa, Homo sapiens
DALPAGWEQRELPNGRVYYVDHNTKTTTWERPLP

SEQ ID NO: 11 - protein, 33 aa, Homo sapiens
PLPPGWEKRTDPRGRFYYVDHNTRTTTWQRPTA

SEQ ID NO: 12 - protein, 37 aa, Homo sapiens
HDPLGPLPPGWEKRQDNGRVYYVNHNTRTTQWEDPRT

SEQ ID NO: 13 - protein, 34 aa, Homo sapiens
PALPPGWEMKYTSEGVRYFVDHNTRTTTFKDPRP

SEQ ID NO: 14 - protein, 1319 aa, Homo sapiens
MAQSLRLHFAARRSNTYPLSETSGDDLDSHVHMCFKRPTRISTSNVVQMKLTPRQTALAPLIKENVQSQERSSVPSSENVNKKSSCLQISLQPTRYSGYLQSSNVLADSDDASFTCILKDGIYSSAVVDNELNAVNDGHLVSSPAICSGSLSNFSTSDNGSYSSNGSDFGSCASITSGGSYTNSVISDSSSYTFPPSDDTFLGGNLPSDSTSNRSVPNRNTTPCEIFSRSTSTDPFVQDDLEHGLEIMKLPVSRNTKIPLKRYSSLVIFPRSPSTTRPTSPTSLCTLLSKGSYQTSHQFIISPSEIAHNEDGTSAKGFLSTAVNGLRLSKTICTPGEVRDIRPLHRKGSLQKKIVLSNNTPRQTVCEKSSEGYSCVSVHFTQRKAATLDCETTNGDCKPEMSEIKLNSDSEYIKLMHRTSACLPSSQNVDCQININGELERPHSQMNKNHGILRRSISLGGAYPNISCLSSLKHNCSKGGPSQLLIKFASGNEGKVDNLSRDSNRDCTNELSNSCKTRDDFLGQVDVPLYPLPTENPRLERPYTFKDFVLHPRSHKSRVKGYLRLKMTYLPKTSGSEDDNAEQAEELEPGWVVLDQPDAACHLQQQQEPSPLPPGWEERQDILGRTYYVNHESRRTQWKRPTPQDNLTDAENGNIQLQAQRAFTTRRQISEETESVDNRESSENWEIIREDEATMYSNQAFPSPPPSSNLDVPTHLAEELNARLTIFGNSAVSQPASSSNHSSRRGSLQAYTFEEQPTLPVLLPTSSGLPPGWEEKQDERGRSYYVDHNSRTTTWTKPTVQATVETSQLTSSQSSAGPQSQASTSDSGQQVTQPSEIEQGFLPKGWEVRHAPNGRPFFIDHNTKTTTWEDPRLKIPAHLRGKTSLDTSNDLGPLPPGWEERTHTDGRIFYINHNIKRTQWEDPRLENVAITGPAVPYSRDYKRKYEFFRRKLKKQNDIPNKFEMKLRRATVLEDSYRRIMGVKRADFLKARLWIEFDGEKGLDYGGVAREWFFLISKEMFNPYYGLFEYSATDNYTLQINPNSGLCNEDHLSYFKFIGRVAGMAVYHGKLLDGFFIRPFYKMMLHKPITLHDMESVDSEYYNSLRWILENDPTELDLRFIIDEELFGQTHQHELKNGGSEIVVTNKNKKEYIYLVIQWRFVNRIQKQMAAFKEGFFELIPQDLIKIFDENELELLMCGLGDVDVNDWREHTKYKNGYSANHQVIQWFWKAVLMMDSEKRIRLLQFVTGTSRVPMNGFAELYGSNGPQSFTVEQWGTPEKLPRAHTCFNRLDLPPYESFEELWDKLQMAIENTQGFDGVD

SEQ ID NO: 15 - protein, 34 aa, Homo sapiens
SPLPPGWEERQDILGRTYYVNHESRRTQWKRPTP

SEQ ID NO: 16 - protein, 34 aa, Homo sapiens
SGLPPGWEEKQDERGRSYYVDHNSRTTTWTKPTV

SEQ ID NO: 17 - protein, 34 aa, Homo sapiens
GFLPKGWEVRHAPNGRPFFIDHNTKTTTWEDPRL

SEQ ID NO: 18 - protein, 34 aa, Homo sapiens
GPLPPGWEERTHTDGRIFYINHNIKRTQWEDPRL

SEQ ID NO: 19 - protein, 955 aa, Homo sapiens
MATGLGEPVYGLSEDEGESRILRVKVVSGIDLAKKDIFGASDPYVKLSLYVADENRELALVQTKTIKKTLNPKWNEEFYFRVNPSNHRLLFEVFDENRLTRDDFLGQVDVPLSHLPTEDPTMERPYTFKDFLLRPRSHKSRVKGFLRLKMAYMPKNGGQDEENSDQRDDMEHGWEVVDSNDSASQHQEELPPPPLPPGWEEKVDNLGRTYYVNHNNRTTQWHRPSLMDVSSESDNNIRQINQEAAHRRFRSRRHISEDLEPEPSEGGDVPEPWETISEEVNIAGDSLGLALPPPPASPGSRTSPQELSEELSRRLQITPDSNGEQFSSLIQREPSSRLRSCSVTDAVAEQGHLPPPSVAYVHTTPGLPSGWEERKDAKGRTYYVNHNNRTTTWTRPIMQLAEDGASGSATNSNNHLIEPQIRRPRSLSSPTVTLSAPLEGAKDSPVRRAVKDTLSNPQSPQPSPYNSPKPQHKVTQSFLPPGWEMRIAPNGRPFFIDHNTKTTTWEDPRLKFPVHMRSKTSLNPNDLGPLPPGWEERIHLDGRTFYIDHNSKITQWEDPRLQNPAITGPAVPYSREFKQKYDYFRKKLKKPADIPNRFEMKLHRNNIFEESYRRIMSVKRPDVLKARLWIEFESEKGLDYGGVAREWFFLLSKEMFNPYYGLFEYSATDNYTLQINPNSGLCNEDHLSYFTFIGRVAGLAVFHGKLLDGFFIRPFYKMMLGKQITLNDMESVDSEYYNSLKWILENDPTELDLMFCIDEENFGQTYQVDLKPNGSEIMVTNENKREYIDLVIQWRFVNRVQKQMNAFLEGFTELLPIDLIKIFDENELELLMCGLGDVDVNDWRQHSIYKNGYCPNHPVIQWFWKAVLLMDAEKRIRLLQFVTGTSRVPMNGFAELYGSNGPQLFTIEQWGSPEKLPRAHTCFNRLDLPPYETFEDLREKLLMAVENAQGFEGVD

SEQ ID NO: 20 - protein, 27 aa, Homo sapiens
GWEEKVDNLGRTYYVNHNNRTTQWHRP

SEQ ID NO: 21 - protein, 29 aa, Homo sapiens
PSGWEERKDAKGRTYYVNHNNRTTTWTRP

SEQ ID NO: 22 - protein, 31 aa, Homo sapiens
PPGWEMRIAPNGRPFFIDHNTKTTTWEDPRL

SEQ ID NO: 23 - protein, 31 aa, Homo sapiens
PPGWEERIHLDGRTFYIDHNSKITQWEDPRL

SEQ ID NO: 24 - protein, 756 aa, Homo sapiens
MSNPGTRRNGSSIKIRLTVLCAKNLAKKDFFRLPDPFAKIVVDGSGQCHSTDTVKNTLDPKWNQHYDLYVGKTDSITISVWNHKKIHKKQGAGFLGCVRLLSNAISRLKDTGYQRLDLCKLNPSDTDAVRGQIVVSLQTRDRIGTGGSVDCRGLLENEGTVYEDSGPGRPLSCFMEEPAPYTDSTGAAAGGGNCRFVESPSQDQRLQAQRLRNPDVRGSLQTPQNRPHGHQSPELPEGYEQRTTVQGQVYFLHTQTGVSTWHDPRIPSPSGTIPGGDAAFLYEFLLQGHTSEPRDLNSVNCDELGPLPPGWEVRSTVSGRIYFVDHNNRTTQFTDPRLHHIMNHQCQLKEPSQPLPLPSEGSLEDEELPAQRYERDLVQKLKVLRHELSLQQPQAGHCRIEVSREEIFEESYRQIMKMRPKDLKKRLMVKFRGEEGLDYGGVAREWLYLLCHEMLNPYYGLFQYSTDNIYMLQINPDSSINPDHLSYFHFVGRIMGLAVFHGHYINGGFTVPFYKQLLGKPIQLSDLESVDPELHKSLVWILENDITPVLDHTFCVEHNAFGRILQHELKPNGRNVPVTEENKKEYVRLYVNWRFMRGIEAQFLALQKGFNELIPQHLLKPFDQKELELIIGGLDKIDLNDWKSNTRLKHCVADSNIVRWFWQAVETFDEERRARLLQFVTGSTRVPLQGFKALQGSTGAAGPRLFTIHLIDANTDNLPKAHTCFNRIDIPPYESYEKLYEKLLTAVEETCGFAVE

SEQ ID NO: 25 - protein, 34 aa, Homo sapiens
PELPEGYEQRTTVQGQVYFLHTQTGVSTWHDPRI

SEQ ID NO: 26 - protein, 34 aa, Homo sapiens
GPLPPGWEVRSTVSGRIYFVDHNNRTTQFTDPRL

SEQ ID NO: 27 - protein, 748 aa, Homo sapiens
MSNPGGRRNGPVKLRLTVLCAKNLVKKDFFRLPDPFAKVVVDGSGQCHSTDTVKNTLDPKWNQHYDLYIGKSDSVTISVWNHKKIHKKQGAGFLGCVRLLSNAINRLKDTGYQRLDLCKLGPNDNDTVRGQIVVSLQSRDRIGTGGQVVDCSRLFDNDLPDGWEERRTASGRIQYLNHITRTTQWERPTRPASEYSSPGRPLSCFVDENTPISGTNGATCGQSSDPRLAERRVRSQRHRNYMSRTHLHTPPDLPEGYEQRTTQQGQVYFLHTQTGVSTWHDPRVPRDLSNINCEELGPLPPGWEIRNTATGRVYFVDHNNRTTQFTDPRLSANLHLVLNRQNQLKDQQQQQVVSLCPDDTECLTVPRYKRDLVQKLKILRQELSQQQPQAGHCRIEVSREEIFEESYRQVMKMRPKDLWKRLMIKFRGEEGLDYGGVAREWLYLLSHEMLNPYYGLFQYSRDDIYTLQINPDSAVNPEHLSYFHFVGRIMGMAVFHGHYIDGGFTLPFYKQLLGKSITLDDMELVDPDLHNSLVWILENDITGVLDHTFCVEHNAYGEIIQHELKPNGKSIPVNEENKKEYVRLYVNWRFLRGIEAQFLALQKGFNEVIPQHLLKTFDEKELELIICGLGKIDVNDWKVNTRLKHCTPDSNIVKWFWKAVEFFDEERRARLLQFVTGSSRVPLQGFKALQGAAGPRLFTIHQIDACTNNLPKAHTCFNRIDIPPYESYEKLYEKLLTAIEETCGFAVE

SEQ ID NO: 28 - protein, 34 aa, Homo sapiens
NDLPDGWEERRTASGRIQYLNHITRTTQWERPTR

SEQ ID NO: 29 - protein, 34 aa, Homo sapiens
PDLPEGYEQRTTQQGQVYFLHTQTGVSTWHDPRV

SEQ ID NO: 30 - protein, 34 aa, Homo sapiens
GPLPPGWEIRNTATGRVYFVDHNNRTTQFTDPRL

SEQ ID NO: 31 - protein, 903 aa, Homo sapiens
MSDSGSQLGSMGSLTMKSQLQITVISAKLKENKKNWFGPSPYVEVTVDGQSKKTEKCNNTNSPKWKQPLTVIVTPVSKLHFRVWSHQTLKSDVLLGTAALDIYETLKSNNMKLEEVVVTLQLGGDKEPTETIGDLSICLDGLQLESEVVTNGETTCSENGVSLCLPRLECNSAISAHCNLCLPGLSDSPISASRVAGFTGASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 32 - protein, 34 aa, Homo sapiens
APLPPGWEQRVDQHGRVYYVDHVEKRTTWDRPEP

SEQ ID NO: 33 - protein, 34 aa, Homo sapiens
EPLPPGWERRVDNMGRIYYVDHFTRTTTWQRPTL

SEQ ID NO: 34 - protein, 34 aa, Homo sapiens
GPLPPGWEKRTDSNGRVYFVNHNTRITQWEDPRS

SEQ ID NO: 35 - protein, 34 aa, Homo sapiens
KPLPEGWEMRFTVDGIPYFVDHNRRTTTYIDPRT

SEQ ID NO: 36 - protein, 1606 aa, Homo sapiens
MLLHLCSVKNLYQNRFLGLAAMASPSRNSQSRRRCKEPLRYSYNPDQFHNMDLRGGPHDGVTIPRSTSDTDLVTSDSRSTLMVSSSYYSIGHSQDLVIHWDIKEEVDAGDWIGMYLIDEVLSENFLDYKNRGVNGSHRGQIIWKIDASSYFVEPETKICFKYYHGVSGALRATTPSVTVKNSAAPIFKSIGADETVQGQGSRRLISFSLSDFQAMGLKKGMFFNPDPYLKISIQPGKHSIFPALPHHGQERRSKIIGNTVNPIWQAEQFSFVSLPTDVLEIEVKDKFAKSRPIIKRFLGKLSMPVQRLLERHAIGDRVVSYTLGRRLPTDHVSGQLQFRFEITSSIHPDDEEISLSTEPESAQIQDSPMNNLMESGSGEPRSEAPESSESWKPEQLGEGSVPDGPGNQSIELSRPAEEAAVITEAGDQGMVSVGPEGAGELLAQVQKDIQPAPSAEELAEQLDLGEEASALLLEDGEAPASTKEEPLEEEATTQSRAGREEEEKEQEEEGDVSTLEQGEGRLQLRASVKRKSRPCSLPVSELETVIASACGDPETPRTHYIRIHTLLHSMPSAQGGSAAEEEDGAEEESTLKDSSEKDGLSEVDTVAADPSALEEDREEPEGATPGTAHPGHSGGHFPSLANGAAQDGDTHPSTGSESDSSPRQGGDHSCEGCDASCCSPSCYSSSCYSTSCYSSSCYSASCYSPSCYNGNRFASHTRFSSVDSAKISESTVFSSQDDEEEENSAFESVPDSMQSPELDPESTNGAGPWQDELAAPSGHVERSPEGLESPVAGPSNRREGECPILHNSQPVSQLPSLRPEHHHYPTIDEPLPPNWEARIDSHGRVFYVDHVNRTTTWQRPTAAATPDGMRRSGSIQQMEQLNRRYQNIQRTIATERSEEDSGSQSCEQAPAGGGGGGGSDSEAESSQSSLDLRREGSLSPVNSQKITLLLQSPAVKFITNPEFFTVLHANYSAYRVFTSSTCLKHMILKVRRDARNFERYQHNRDLVNFINMFADTRLELPRGWEIKTDQQGKSFFVDHNSRATTFIDPRIPLQNGRLPNHLTHRQHLQRLRSYSAGEASEVSRNRGASLLARPGHSLVAAIRSQHQHESLPLAYNDKIVAFLRQPNIFEMLQERQPSLARNHTLREKIHYIRTEGNHGLEKLSCDADLVILLSLFEEEIMSYVPLQAAFHPGYSFSPRCSPCSSPQNSPGLQRASARAPSPYRRDFEAKLRNFYRKLEAKGFGQGPGKIKLIIRRDHLLEGTFNQVMAYSRKELQRNKLYVTFVGEEGLDYSGPSREFFFLLSQELFNPYYGLFEYSANDTYTVQISPMSAFVENHLEWFRFSGRILGLALIHQYLLDAFFTRPFYKALLRLPCDLSDLEYLDEEFHQSLQWMKDNNITDILDLTFTVNEEVFGQVTERELKSGGANTQVTEKNKKEYIERMVKWRVERGVVQQTEALVRGFYEVVDSRLVSVFDARELELVIAGTAEIDLNDWRNNTEYRGGYHDGHLVIRWFWAAVERFNNEQRLRLLQFVTGTSSVPYEGFAALRGSNGLRRFCIEKWGKITSLPRAHTCFNRLDLPPYPSYSMLYEKLLTAVEETSTFGLE

SEQ ID NO: 37 - protein, 33 aa, Homo sapiens
PLPPNWEARIDSHGRVFYVDHVNRTTTWQRPTA

SEQ ID NO: 38 - protein, 34 aa, Homo sapiens
LELPRGWEIKTDQQGKSFFVDHNSRATTFIDPRI

SEQ ID NO: 39 - protein, 1572 aa, Homo sapiens
MASSAREHLLFVRRRNPQMRYTLSPENLQSLAAQSSMPENMTLQRANSDTDLVTSESRSSLTASMYEYTLGQAQNLIIFWDIKEEVDPSDWIGLYHIDENSPANFWDSKNRGVTGTQKGQIVWRIEPGPYFMEPEIKICFKYYHGISGALRATTPCITVKNPAVMMGAEGMEGGASGNLHSRKLVSFTLSDLRAVGLKKGMFFNPDPYLKMSIQPGKKSSFPTCAHHGQERRSTIISNTTNPIWHREKYSFFALLTDVLEIEIKDKFAKSRPIIKRFLGKLTIPVQRLLERQAIGDQMLSYNLGRRLPADHVSGYLQFKVEVTSSVHEDASPEAVGTILGVNSVNGDLGSPSDDEDMPGSHHDSQVCSNGPVSEDSAADGTPKHSFRTSSTLEIDTEELTSTSSRTSPPRGRQDSLNDYLDAIEHNGHSRPGTATCSERSMGASPKLRSSFPTDTRLNAMLHIDSDEEDHEFQQDLGYPSSLEEEGGLIMFSRASRADDGSLTSQTKLEDNPVENEEASTHEAASFEDKPENLPELAESSLPAGPAPEEGEGGPEPQPSADQGSAELCGSQEVDQPTSGADTGTSDASGGSRRAVSETESLDQGSEPSQVSSETEPSDPARTESVSEASTRPEGESDLECADSSCNESVTTQLSSVDTRCSSLESARFPETPAFSSQEEEDGACAAEPTSSGPAEGSQESVCTAGSLPVVQVPSGEDEGPGAESATVPDQEELGEVWQRRGSLEGAAAAAESPPQEEGSAGEAQGTCEGATAQEEGATGGSQANGHQPLRSLPSVRQDVSRYQRVDEALPPNWEARIDSHGRIFYVDHVNRTTTWQRPTAPPAPQVLQRSNSIQQMEQLNRRYQSIRRTMTNERPEENTNAIDGAGEEADFHQASADFRRENILPHSTSRSRITLLLQSPPVKFLISPEFFTVLHSNPSAYRMFTNNTCLKHMITKVRRDTHHFERYQHNRDLVGFLNMFANKQLELPRGWEMKHDHQGKAFFVDHNSRTTTFIDPRLPLQSSRPTSALVHRQHLTRQRSHSAGEVGEDSRHAGPPVLPRPSSTFNTVSRPQYQDMVPVAYNDKIVAFLRQPNIFEILQERQPDLTRNHSLREKIQFIRTEGTPGLVRLSSDADLVMLLSLFEEEIMSYVPPHALLHPSYCQSPRGSPVSSPQNSPGTQRANARAPAPYKRDFEAKLRNFYRKLETKGYGQGPGKLKLIIRRDHLLEDAFNQIMGYSRKDLQRNKLYVTFVGEEGLDYSGPSREFFFLVSRELFNPYYGLFEYSANDTYTVQISPMSAFVDNHHEWFRFSGRILGLALIHQYLLDAFFTRPFYKALLRILCDLSDLEYLDEEFHQSLQWMKDNDIHDILDLTFTVNEEVFGQITERELKPGGANIPVTEKNKKEYIERMVKWRIERGVVQQTESLVRGFYEVVDARLVSVFDARELELVIAGTAEIDLSDWRNNTEYRGGYHDNHIVIRWFWAAVERFNNEQRLRLLQFVTGTSSIPYEGFASLRGSNGPRRFCVEKWGKITALPRAHTCFNRLDLPPYPSFSMLYEKLLTAVEETSTFGLE

SEQ ID NO: 40 - protein, 34 aa, Homo sapiens
EALPPNWEARIDSHGRIFYVDHVNRTTTWQRPTA

SEQ ID NO: 41 - protein, 34 aa, Homo sapiens
LELPRGWEMKHDHQGKAFFVDHNSRTTTFIDPRL

SEQ ID NO: 42 - protein, 433 aa, Homo sapiens
MGRVQLFEISLSHGRVVYSPGEPLAGTVRVRLGAPLPFRAIRVTCIGSCGVSNKANDTAWVVEEGYFNSSLSLADKGSLPAGEHSFPFQFLLPATAPTSFEGPFGKIVHQVRAAIHTPRFSKDHKCSLVFYILSPLNLNSIPDIEQPNVASATKKFSYKLVKTGSVVLTASTDLRGYVVGQALQLHADVENQSGKDTSPVVASLLQKVSYKAKRWIHDVRTIAEVEGAGVKAWRRAQWHEQILVPALPQSALPGCSLIHIDYYLQVSLKAPEATVTLPVFIGNIAVNHAPVSPRPGLGLPPGAPPLVVPSAPPQEEAEAEAAAGGPHFLDPVFLSTKSHSQRQPLLATLSSVPGAPEPCPQDGSPASHPLHPPLCISTGATVPYFAEGSGGPVPTTSTLILPPEYSSWGYPYEAPPSYEQSCGGVEPSLTPES

SEQ ID NO: 43 - protein, 434 aa, Mus musculus
MGRVQLFEIRLSQGRVVYGPGEPLAGTVHLRLGAPLPFRAIRVTCMGSCGVSTKANDGAWVVEESYFNSSLSLADKGSLPAGEHNFPFQFLLPATAPTSFEGPFGKIVHQVRASIDTPRFSKDHKCSLVFYILSPLNLNSIPDIEQPNVASTTKKFSYKLVKTGNVVLTASTDLRGYVVGQVLRLQADIENQSGKDTSPVVASLLQKVSYKAKRWIYDVRTIAEVEGTGVKAWRRAQWQEQILVPALPQSALPGCSLIHIDYYLQVSMKAPEATVTLPLFVGNIAVNQTPLSPCPGRESSPGTLSLVVPSAPPQEEAEAVASGPHFSDPVSLSTKSHSQQQPLSAPLGSVSVTTTEPWVQVGSPARHSLHPPLCISIGATVPYFAEGSAGPVPTTSALILPPEYSSWGYPYEAPPSYEQSCGAAGTDLGLIPGS

SEQ ID NO: 44 - protein, 433 aa, Mus musculus
MGRVQLFEIRLSQGRVVYGPGEPLAGTVHLRLGAPLPFRAIRVTCMGSCGVSTKANDGAWVVEESYFNSSLSLADKGSLPAGEHNFPFQFLLPATAPTSFEGPFGKIVHQVRASIDTPRFSKDHKCSLVFYILSPLNLNSIPDIEQPNVASTTKKFSYKLVKTGNVVLTASTDLRGYVVGQVLRLQADIENQSGKDTSPVVASLLQVSYKAKRWIYDVRTIAEVEGTGVKAWRRAQWQEQILVPALPQSALPGCSLIHIDYYLQVSMKAPEATVTLPLFVGNIAVNQTPLSPCPGRESSPGTLSLVVPSAPPQEEAEAVASGPHFSDPVSLSTKSHSQQQPLSAPLGSVSVTTTEPWVQVGSPARHSLHPPLCISIGATVPYFAEGSAGPVPTTSALILPPEYSSWGYPYEAPPSYEQSCGAAGTDLGLIPGS

SEQ ID NO: 45 - protein, 390 aa, Homo sapiens
MAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRPISASYPPYQATGPPNTSYMPGMPGGISPYPSGYPPNPSGYPGCPYPPGGPYPATTSSQYPSQPPVTTVGPSRDGTISEDTIRASLISAVSDKLRWRMKEEMDRAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 46 - protein, 391 aa, Mus musculus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTATGPPNTSYMPGMPSGISAYPSGYPPNPSGYPGCPYPPAGPYPATTSSQYPSQPPVTTVGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 47 - protein, 391 aa, Rattus norvegicus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTAAGPPNTSYLPSMPSGISAYPSGYPPNPSGYPGCPYPPAGPYPATTSSQYPSQPPVTTAGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 48 - DNA, 3475 bp, Homo sapiens
  source 1..3475: mol_type = genomic DNA
gaattcgcggccgcgtcgaccgcttctgtggccacggcagatgaaacagaaaggctaaagagggctggagtcaggggacttctcttccaccagcttcacggtgatgatatggcatctgccagctctagccgggcaggagtggccctgccttttgagaagtctcagctcactttgaaagtggtgtccgcaaagcccaaggtgcataatcgtcaacctcgaattaactcctacgtggaggtggcggtggatggactccccagtgagaccaagaagactgggaagcgcattgggagctctgagcttctctggaatgagatcatcattttgaatgtcacggcacagagtcatttagatttaaaggtctggagctgccataccttgagaaatgaactgctaggcaccgcatctgtcaacctctccaacgtcttgaagaacaatgggggcaaaatggagaacatgcagctgaccctgaacctgcagacggagaacaaaggcagcgttgtctcaggcggaaaactgacaattttcctggacgggccaactgttgatctgggaaatgtgcctaatggcagtgccctgacagatggatcacagctgccttcgagagactccagtggaacagcagtagctccagagaaccggcaccagccccccagcacaaactgctttggtggaagatcccggacgcacagacattcgggtgcttcagccagaacaaccccagcaaccggcgagcaaagccccggtgctcggagccggcaccgccagcccgtcaagaactcaggccacagtggcttggccaatggcacagtgaatgatgaacccacaacagccactgatcccgaagaaccttccgttgttggtgtgacgtccccacctgctgcacccttgagtgtgaccccgaatcccaacacgacttctctccctgccccagccacaccggctgaaggagaggaacccagcacttcgggtacacagcagctcccagcggctgcccaggcccccgacgctctgcctgctggatgggaacagcgagagctgcccaacggacgtgtctattatgttgaccacaataccaagaccaccacctgggagcggccccttcctccaggctgggaaaaacgcacagatccccgaggcaggttttactatgtggatcacaatactcggaccaccacctggcagcgtccgaccgcggagtacgtgcgcaactatgagcagtggcagtcgcagcggaatcagctccagggggccatgcagcacttcagccaaagattcctataccagttttggagtgcttcgactgaccatgatcccctgggccccctccctcctggttgggagaaaagacaggacaatggacgggtgtattacgtgaaccataacactcgcacgacccagtgggaggatccccggacccaggggatgatccaggaaccagctttgcccccaggatgggagatgaaatacaccagcgagggggtgcgatactttgtggaccacaatacccgcaccaccacctttaaggatcctcgcccggggtttgagtcggggacgaagcaaggttcccctggtgcttatgaccgcagttttcggtggaagtatcaccagttccgtttcctctgccattcaaatgccctacctagccacgtgaagatcagcgtttccaggcagacgcttttcgaagattccttccaacagatcatgaacatgaaaccctatgacctgcgccgccggctttacatcatcatgcgtggcgaggagggcctggactatgggggcatcgccagagagtggtttttcctcctgtctcacgaggtgctcaaccctatgtattgtttatttgaatatgccggaaagaacaattactgcctgcagatcaaccccgcctcctccatcaacccggaccacctcacctactttcgctttataggcagattcatcgccatggcgctgtaccatggaaagttcatcgacacgggcttcaccctccctttctacaagcggatgctcaataagagaccaaccctgaaagacctggagtccattgaccctgagttctacaactccattgtctggatcaaagagaacaacctggaagaatgtggcctggagctgtacttcatccaggacatggagatactgggcaaggtgacgacccacgagctgaaggagggcggcgagagcatccgggtcacggaggagaacaaggaagagtacatcatgctgctgactgactggcgtttcacccgaggcgtggaagagcagaccaaagccttcctggatggcttcaacgaggtggccccgctggagtggctgcgctactttgacgagaaagagctggagctgatgctgtgcggcatgcaggagatagacatgagcgactggcagaagagcaccatctaccggcactacaccaagaacagcaagcagatccagtggttctggcaggtggtgaaggagatggacaacgagaagaggatccggctgctgcagtttgtcaccggtacctgccgcctgcccgtcgggggatttgccgaactcatcggtagcaacggaccacagaagttttgcattgacaaagttggcaaggaaacctggctgcccagaagccacacctgcttcaaccgtctggatcttccaccctacaagagctacgaacagctgagagagaagctgctgtatgccattgaggagaccgagggctttggacaggagtaaccgaggccgcccctcccacgccccccagcgcacatgtagtcctgagtcctccctgcctgagaggccactggccccgcagcccttgggaggcccccgtggatgtggccctgtgtgggaccacactgtcatctcgctgctggcagaaaagcctgatcccaggaggccctgcagttcccccgacccgcggatggcagtctggaataaagccccctagttgcctttggccccacctttgcaaagttccagagggctgaccctctctgcaaaactctcccctgtcctctagaccccaccctgggtgtatgtgagtgtgcaagggaaggtgttgcatccccaggggctgccgcagaggccggagacctcctggactagttcggcgaggagactggccactgggggtggctgttcgggactgagagcgccaagggtctttgccagcaaaggaggttctgcctgtaattgagcctctctgatgatggagatgaagtgaaggtctgagggacgggccctggggctaggccatctctgcctgcctccctagcaggcgccagcggtggaggctgagtcgcaggacacatgccggccagttaattcattctcagcaaatgaaggtttgtctaagctgcctgggtatccacgggacaaaaacagcaaactccctccagactttgtccatgttataaacttgaaagttggttgttgtttgttaggtttgccaggtttttttgtttacgcctgctgtcactttcctgtc

SEQ ID NO: 49 - DNA, 3475 bp, Homo sapiens; identical to SEQ ID NO: 48
  source 1..3475: mol_type = genomic DNA

SEQ ID NO: 50 - DNA, 2914 bp, Homo sapiens
  source 1..2914: mol_type = genomic DNA
acagttgcctgccctgggcgggggcgagcgcgtccggtttgctggaagcgttcggaaatggcaacttgcgcggtggaggtgttcgggctcctggaggacgaggaaaattcacgaattgtgagagtaagagttatagccggaataggccttgccaagaaggatatattgggagctagtgatccttacgtgagagtgacgttatatgacccaatgaatggagttcttacaagtgtgcaaacaaaaaccattaaaaagagtttgaatccaaagtggaatgaagaaatattattcagagttcatcctcagcagcaccggcttctttttgaagtgtttgacgaaaaccgattgacaagagatgatttcctaggtcaagtggatgttccactttatccattaccgacagaaaatccaagattggagagaccatatacatttaaggattttgttcttcatccaagaagtcacaaatcaagagttaaaggttatctgagactaaaaatgacttatttacctaaaaccagtggctcagaagatgataatgcagaacaggctgaggaattagagcctggctgggttgttttggaccaaccagatgctgcttgccatttgcagcaacaacaagaaccttctcctctacctccagggtgggaagagaggcaggatatccttggaaggacctattatgtaaaccatgaatctagaagaacacagtggaaaagaccaacccctcaggacaacctaacagatgctgagaatggcaacattcaactgcaagcacaacgtgcatttaccaccaggcggcagatatccgaggaaacagaaagtgttgacaaccaagagtcttccgagaactgggaaattataagagaagatgaagccaccatgtatagcagccaggccttcccatcacctccaccgtcaagtaacttggatgttccaactcatcttgcagaagaattgaatgccagactcaccatttttggaaattcagccgtgagccagccagcatcgagctcaaatcattccagcagaagaggcagcttacaagcctatacttttgaggaacaacctacacttcctgtgcttttgcctacttcatctggattaccaccaggttgggaagaaaaacaagatgaaagaggaagatcatattatgtagatcacaattccagaacgactacttggacaaagcccactgtacaggccacagtggagaccagtcagctgacctcaagccagagttctgcaggccctcaatcacaagcctccaccagtgattcaggccagcaggtgacccagccatctgaaattgagcaaggattccttcctaaaggctgggaagtccggcatgcaccaaatgggaggcctttctttattgaccacaacactaaaaccaccacctgggaagatccaagattgaaaattccagcccatctgagaggaaagacatcacttgatacttccaatgatctagggcctttacctccaggatgggaagagagaactcacacagatggaagaatcttctacataaatcacaatataaaaagaacacaatgggaagatcctcggttggagaatgtagcaataactggaccagcagtgccctactccagggattacaaaagaaagtatgagttcttccgaagaaagttgaagaagcagaatgacattccaaacaaatttgaaatgaaacttcgccgagcaactgttcttgaagactcttaccggagaattatgggtgtcaagagagcagacttcctgaaggctcgactgtggattgagtttgatggtgaaaagggattggattatggaggagttgccagagaatggttcttcctgatctcaaaggaaatgtttaacccttattatgggttgtttgaatattctgctacggacaattataccctacagataaatccaaactctggattgtgtaacgaagatcacctctcttacttcaagtttattggtcgggtagctggaatggcagtttatcatggcaaactgttggatggttttttcatccgcccattttacaagatgatgcttcacaaaccaataacccttcatgatatggaatctgtggatagtgaatattacaattccctaagatggattcttgaaaatgacccaacagaattggacctcaggtttatcatagatgaagaactttttggacagacacatcaacatgagctgaaaaatggtggatcagaaatagttgtcaccaataagaacaaaaaggaatatatttatcttgtaatacaatggcgatttgtaaaccgaatccagaagcaaatggctgcttttaaagagggattctttgaactaataccacaggatctcatcaaaatttttgatgaaaatgaactagagcttcttatgtgtggaccgggagatgttgatgtgaatgactggagggaacatacaaagtataaaaatggctacagtgcaaatcatcaggttatacagtggttttggaaggctgttttaatgatggattcagaaaaaagaataagattacttcagtttgtcactggcacatctcgggtgcctatgaatggatttgctgaactatacggttcaaatggaccacagtcatttacagttgaacagtggggtactcctgaaaagctgccaagagctcatacctgttttaatcgcctggacttgccaccttatgaatcatttgaagaattatgggataaacttcagatggcaattgaaaacacccagggctttgatggagttgattagattacaaataacaatctgtagtgtttttactgccatagttttataaccaaaatcttgacttaaaattttccggggaactactaaaatgtggccactgagtcttcccagatcttgaagaaaatcatataaaaagcatttgaagaaatagtacgac

SEQ ID NO: 51 - RNA, 2868 nt, Homo sapiens
  source 1..2868: mol_type = mRNA
atggcgaccgggctcggggagccggtctatggactttccgaagacgagggagagtcccgtattctcagagtaaaagttgtttctggaattgatctcgccaaaaaggacatctttggagccagtgatccgtatgtgaaactttcattgtacgtagcggatgagaatagagaacttgctttggtccagacaaaaacaattaaaaagacactgaacccaaaatggaatgaagaattttatttcagggtaaacccatctaatcacagactcctatttgaagtatttgacgaaaatagactgacacgagacgacttcctgggccaggtggacgtgccccttagtcaccttccgacagaagatccaaccatggagcgaccctatacatttaaggactttctcctcagaccaagaagtcataagtctcgagttaagggatttttgcgattgaaaatggcctatatgccaaaaaatggaggtcaagatgaagaaaacagtgaccagagggatgacatggagcatggatgggaagttgttgactcaaatgactcggcttctcagcaccaagaggaacttcctcctcctcctctgcctcccgggtgggaagaaaaagtggacaatttaggccgaacttactatgtcaaccacaacaaccggaccactcagtggcacagaccaagcctgatggacgtgtcctcggagtcggacaataacatcagacagatcaaccaggaggcagcacaccggcgcttccgctcccgcaggcacatcagcgaagacttggagcccgagccctcggagggcggggatgtccccgagccttgggagaccatttcagaggaagtgaatatcgctggagactctctcggtctggctctgcccccaccaccggcctccccaggatctcggaccagccctcaggagctgtcagaggaactaagcagaaggcttcagatcactccagactccaatggggaacagttcagctctttgattcaaagagaaccctcctcaaggttgaggtcatgcagtgtcaccgacgcagttgcagaacagggccatctaccaccgccatcagtggcctatgtacataccacgccgggtctgccttcaggctgggaagaaagaaaagatgctaaggggcgcacatactatgtcaatcataacaatcgaaccacaacttggactcgacctatcatgcagcttgcagaagatggtgcgtccggatcagccacaaacagtaacaaccatctaatcgagcctcagatccgccggcctcgtagcctcagctcgccaacagtaactttatctgccccgctggagggtgccaaggactcacccgtacgtcgggctgtgaaagacaccctttccaacccacagtccccacagccatcaccttacaactcccccaaaccacaacacaaagtcacacagagcttcttgccacccggctgggaaatgaggatagcgccaaacggccggcccttcttcattgatcataacacaaagactacaacctgggaagatccacgtttgaaatttccagtacatatgcggtcaaagacatctttaaaccccaatgaccttggcccccttcctcctggctgggaagaaagaattcacttggatggccgaacgttttatattgatcataatagcaaaattactcagtgggaagacccaagactgcagaacccagctattactggtccggctgtcccttactccagagaatttaagcagaaatatgactacttcaggaagaaattaaagaaacctgctgatatccccaataggtttgaaatgaaacttcacagaaataacatatttgaagagtcctatcggagaattatgtccgtgaaaagaccagatgtcctaaaagctagactgtggattgagtttgaatcagagaaaggtcttgactatgggggtgtggccagagaatggttcttcttactgtccaaagagatgttcaacccctactacggcctctttgagtactctgccacggacaactacacccttcagatcaaccctaattcaggcctctgtaatgaggatcatttgtcctacttcacttttattggaagagttgctggtctggccgtatttcatgggaagctcttagatggtttcttcattagaccattttacaagatgatgttgggaaagcagataaccctgaatgacatggaatctgtggatagtgaatattacaactctttgaaatggatcctggagaatgaccctactgagctggacctcatgttctgcatagacgaagaaaactttggacagacatatcaagtggatttgaagcccaatgggtcagaaataatggtcacaaatgaaaacaaaagggaatatatcgacttagtcatccagtggagatttgtgaacagggtccagaagcagatgaacgccttcttggagggattcacagaactacttcctattgatttgattaaaatttttgatgaaaatgagctggagttgctcatgtgcggcctcggtgatgtggatgtgaatgactggagacagcattctatttacaagaacggctactgcccaaaccaccccgtcattcagtggttctggaaggctgtgctactcatggacgccgaaaagcgtatccggttactgcagtttgtcacagggacatcgcgagtacctatgaatggatttgccgaactttatggttccaatggtcctcagctgtttacaatagagcaatggggcagtcctgagaaactgcccagagctcacacatgctttaatcgccttgacttacctccatatgaaacctttgaagatttacgagagaaacttctcatggccgtggaaaatgctcaaggatttgaaggggtggattaa

SEQ ID NO: 52 - DNA, 2274 bp, Homo sapiens
  source 1..2274: mol_type = genomic DNA
atgtcgaaccccgggacacgcaggaacggctccagcatcaagatccgtctgacagtgttatgtgccaagaaccttgcaaagaaagacttcttcaggctccctgacccttttgcaaagattgtcgtggatgggtctgggcagtgccactcaaccgacactgtgaaaaacacattggacccaaagtggaaccagcactatgatctatatgttgggaaaacggattcgataaccattagcgtgtggaaccataagaaaattcacaagaaacagggagctggcttcctgggctgtgtgcggctgctctccaatgccatcagcagattaaaagataccggataccagcgtttggatctatgcaaactaaacccctcagatactgatgcagttcgtggccagatagtggtcagtttacagacacgagacagaataggaaccggcggctcggtggtggactgcagaggactgttagaaaatgaaggaacggtgtatgaagactccgggcctgggaggccgctcagctgcttcatggaggaaccagccccttacacagatagcaccggtgctgctgctggaggagggaattgcaggttcgtggagtccccaagtcaagatcaaagacttcaggcacagcggcttcgaaaccctgatgtgcgaggttcactacagacgccccagaaccgaccacacggccaccagtccccggaactgcccgaaggctacgaacaaagaacaacagtccagggccaagtttactttttgcatacacagactggagttagcacgtggcacgaccccaggataccaagtccctcggggaccattcctgggggagatgcagcttttctatacgaattccttctacaaggccatacatctgagcccagagaccttaacagtgtgaactgtgatgaacttggaccactgccgccaggctgggaagtcagaagtacagtttctgggaggatatattttgtagatcataataaccgaacaacccagtttacagacccaaggttacaccacatcatgaatcaccagtgccaactcaaggagcccagccagccgctgccactgcccagtgagggctctctggaggacgaggagcttcctgcccagagatacgaaagagatctagtccagaagctgaaagtcctcagacacgaactgtcgcttcagcagccccaagctggtcattgccgcatcgaagtgtccagagaagaaatctttgaggagtcttaccgccagataatgaagatgcgaccgaaagacttgaaaaaacggctgatggtgaaattccgtggggaagaaggtttggattacggtggtgtggccagggagtggctttacttgctgtgccatgaaatgctgaatccttattacgggctcttccagtattctacggacaatatttacatgttgcaaataaatccggattcttcaatcaaccccgaccacttgtcttatttccactttgtggggcggatcatggggctggctgtgttccatggacactacatcaacgggggcttcacagtgcccttctacaagcagctgctggggaagcccatccagctctcagatctggaatctgtggacccagagctgcataagagcttggtgtggatcctagagaacgacatcacgcctgtactggaccacaccttctgcgtggaacacaacgccttcgggcggatcctgcagcatgaactgaaacccaatggcagaaatgtgccagtcacagaggagaataagaaagaatacgtccggttgtatgtaaactggaggtttatgagaggaatcgaagcccagttcttagctctgcagaaggggttcaatgagctcatccctcaacatctgctgaagccttttgaccagaaggaactggagctgatcataggcggcctggataaaatagacttgaacgactggaagtcgaacacgcggctgaagcactgtgtggccgacagcaacatcgtgcggtggttctggcaagcggtggagacgttcgatgaagaaaggagggccaggctcctgcagtttgtgactgggtccacgcgagtcccgctccaaggcttcaaggctttgcaaggttctacaggcgcggcagggccccggctgttcaccatccacctgatagacgcgaacacagacaaccttccgaaggcccatacctgctttaaccggatcgacattccaccatatgagtcctatgagaagctctacgagaagctgctgacagccgtggaggagacctgcgggtttgctgtggagtga

SEQ ID NO: 53 - DNA, 2247 bp, Homo sapiens
  source 1..2247: mol_type = genomic DNA
atgtctaaccccggacgccggaggaacgggcccgtcaagctgcgcctgacagtactctgtgcaaaaaacctggtgaaaaaggattttttccgacttcctgatccatttgctaaggtggtggttgatggatctgggcaatgccattctacagatactgtgaagaatacgcttgatccaaagtggaatcagcattatgacctgtatattggaaagtctgattcagttacgatcagtgtatggaatcacaagaagatccataagaaacaaggtgctggatttctcggttgtgttcgtcttctttccaatgccatcaaccgcctcaaagacactggttatcagaggttggatttatgcaaactcgggccaaatgacaatgatacagttagaggacagatagtagtaagtcttcagtccagagaccgaataggcacaggaggacaagttgtggactgcagtcgtttatttgataacgatttaccagacggctgggaagaaaggagaaccgcctctggaagaatccagtatctaaaccatataacaagaactacgcaatgggagcgcccaacacgaccggcatccgaatattctagccctggcagacctcttagctgctttgttgatgagaacactccaattagtggaacaaatggtgcaacatgtggacagtcttcagatcccaggctggcagagaggagagtcaggtcacaacgacatagaaattacatgagcagaacacatttacatactcctccagacctaccagaaggctatgaacagaggacaacgcaacaaggccaggtgtatttcttacatacacagactggtgtgagcacatggcatgatccaagagtgcccagggatcttagcaacatcaattgtgaagagcttggtccattgcctcctggatgggagatccgtaatacggcaacaggcagagtttatttcgttgaccataacaacagaacaacacaatttacagatcctcggctgtctgctaacttgcatttagttttaaatcggcagaaccaattgaaagaccaacagcaacagcaagtggtatcgttatgtcctgatgacacagaatgcctgacagtcccaaggtacaagcgagacctggttcagaaactaaaaattttgcggcaagaactttcccaacaacagcctcaggcaggtcattgccgcattgaggtttccagggaagagatttttgaggaatcatatcgacaggtcatgaaaatgagaccaaaagatctctggaagcgattaatgataaaatttcgtggagaagaaggccttgactatggaggcgttgccagggaatggttgtatctcttgtcacatgaaatgttgaatccatactatggcctcttccagtattcaagagatgatatttatacattgcagatcaatcctgattctgcagttaatccggaacatttatcctatttccactttgttggacgaataatgggaatggctgtgtttcatggacattatattgatggtggtttcacattgcctttttataagcaattgcttgggaagtcaattaccttggatgacatggagttagtagatccggatcttcacaacagtttagtgtggatacttgagaatgatattacaggtgttttggaccataccttctgtgttgaacataatgcatatggtgaaattattcagcatgaacttaaaccaaatggcaaaagtatccctgttaatgaagaaaataaaaaagaatatgtcaggctctatgtgaactggagatttttacgaggcattgaggctcaattcttggctctgcagaaaggatttaatgaagtaattccacaacatctgctgaagacatttgatgagaaggagttagagctcattatttgtggacttggaaagatagatgttaatgactggaaggtaaacacccggttaaaacactgtacaccagacagcaacattgtcaaatggttctggaaagctgtggagttttttgatgaagagcgacgagcaagattgcttcagtttgtgacaggatcctctcgagtgcctctgcagggcttcaaagcattgcaaggtgctgcaggcccgagactctttaccatacaccagattgatgcctgcactaacaacctgccgaaagcccacacttgcttcaatcgaatagacattccaccctatgaaagctatgaaaagctatatgaaaagctgctaacagccattgaagaaacatgtggatttgctgtggaatga

SEQ ID NO: 54 - DNA, 2869 bp, Homo sapiens
  source 1..2869: mol_type = genomic DNA
ggagtcgccgccgccccgagttccggtaccatgcatttcacggtggccttgtggagacaacgccttaacccaaggaagtgactcaaactgtgagaactccaggttttccaacctattggtggtatgtctgacagtggatcacaacttggttcaatgggtagcctcaccatgaaatcacagcttcagatcactgtcatctcagcaaaacttaaggaaaataagaagaattggtttggaccaagtccttacgtagaggtcacagtagatggacagtcaaagaagacagaaaaatgcaacaacacaaacagtcccaagtggaagcaaccccttacagttatcgttacccctgtgagtaaattacattttcgtgtgtggagtcaccagacactgaaatctgatgttttgttgggaactgctgcattagatatttatgaaacattaaagtcaaacaatatgaaacttgaagaagtagttgtgactttgcagcttggaggtgacaaagagccaacagagacaataggagacttgtcaatttgtcttgatgggctacagttagagtctgaagttgttaccaatggtgaaactacatgttcagaaagtgcttctcagaatgatgatggctccagatccaaggatgaaacaagagtgagcacaaatggatcagatgaccctgaagatgcaggagctggtgaaaataggagagtcagtgggaataattctccatcactctcaaatggtggttttaaaccttctagacctccaagaccttcacgaccaccaccacccaccccacgtagaccagcatctgtcaatggttcaccatctgccacttctgaaagtgatgggtctagtacaggctctctgccgccgacaaatacaaatacaaatacatctgaaggagcaacatctggattaataattcctcttactatatctggaggctcaggccctaggccattaaatcctgtaactcaagctcccttgccacctggttgggagcagagagtggaccagcacgggcgagtttactatgtagatcatgttgagaaaagaacaacatgggatagaccagaacctctacctcctggctgggaacggcgggttgacaacatgggacgtatttattatgttgaccatttcacaagaacaacaacgtggcagaggccaacactggaatccgtccggaactatgaacaatggcagctacagcgtagtcagcttcaaggagcaatgcagcagtttaaccagagattcatttatgggaatcaagatttatttgctacatcacaaagtaaagaatttgatcctcttggtccattgccacctggatgggagaagagaacagacagcaatggcagagtatatttcgtcaaccacaacacacgaattacacaatgggaagaccccagaagtcaaggtcaattaaatgaaaagcccttacctgaaggttgggaaatgagattcacagtggatggaattccatattttgtggaccacaatagaagaactaccacctatatagatccccgcacaggaaaatctgccctagacaatggacctcagatagcctatgttcgggacttcaaagcaaaggttcagtatttccggttctggtgtcagcaactggccatgccacagcacataaagattacagtgacaagaaaaacattgtttgaggattcctttcaacagataatgagcttcagtccccaagatctgcgaagacgtttgtgggtgatttttccaggagaagaaggtttagattatggaggtgtagcaagagaatggttctttcttttgtcacatgaagtgttgaacccaatgtattgcctgtttgaatatgcagggaaggataactactgcttgcagataaaccccgcttcttacatcaatccagatcacctgaaatattttcgttttattggcagatttattgccatggctctgttccatgggaaattcatagacacgggtttttctttaccattctataagcgtatcttgaacaaaccagttggactcaaggatttagaatctattgatccagaattttacaattctctcatctgggttaaggaaaacaatattgaggaatgtgatttggaaatgtacttctccgttgacaaagaaattctaggtgaaattaagagtcatgatctgaaacctaatggtggcaatattcttgtaacagaagaaaataaagaggaatacatcagaatggtagctgagtggaggttgtctcgaggtgttgaagaacagacacaagctttctttgaaggctttaatgaaattcttccccagcaatatttgcaatactttgatgcaaaggaattagaggtccttttatgtggaatgcaagagattgatttgaatgactggcaaagacatgccatctaccgtcattatgcaaggaccagcaaacaaatcatgtggttttggcagtttgttaaagaaattgataatgagaagagaatgagacttctgcagtttgttactggaacctgccgattgccagtaggaggatttgctgatctcatggggagcaatggaccacagaaattctgcattgaaaaagttgggaaagaaaattggctacccagaagtcatacctgttttaatcgcctggacctgccaccatacaagagctatgagcaactgaaggaaaagctgttgtttgccatagaagaaacagaaggatttggacaagagtaacttctgagaacttgcaccatgaatgggcaagaacttatttgcaatgtttgtccttctctgcctgttgcacatcttgtaaaattggacaatggctctttagagagttatctgagtgtaagtaaattaatgttctcatttaaaaaaaaaaaaaaaaaaa

SEQ ID NO: 55 - DNA, 5169 bp, Homo sapiens
  source 1..5169: mol_type = genomic DNA
gcgcatcaggcgctgttgttggagccggaacaccgtgcgactctgaccgaaccggccccctcctcgcgcacacactcgccgagccgcgcgcgcccctccgccgtgacagtggccgtggcctccgctctctcggggcacccggcagccagagcgcagcgagagcgggcggtcgccagggtcccctccccagccagtcccaggcgcccggtgcactatgcggggcacgtgcgccccccagctctaatctgcgcgctgacaggagcatgatctgtgcccaggccagggctgccaaggaattgatgcgcgtacacgtggtgggtcattatgctgctacacctgtgtagtgtgaagaatctgtaccagaacaggtttttaggcctggccgccatggcgtctccttctagaaactcccagagccgacgccggtgcaaggagccgctccgatacagctacaaccccgaccagttccacaacatggacctcaggggcggcccccacgatggcgtcaccattccccgctccaccagcgacactgacctggtcacctcggacagccgctccacgctcatggtcagcagctcctactattccatcgggcactctcaggacctggtcatccactgggacataaaggaggaagtggacgctggggactggattggcatgtacctcattgatgaggtcttgtccgaaaactttctggactataaaaaccgtggagtcaatggttctcatcggggccagatcatctggaagatcgatgccagctcgtactttgtggaacctgaaactaagatctgcttcaaatactaccatggagtgagtggggccctgcgagcaaccacccccagtgtcacggtcaaaaactcggcagctcctatttttaaaagcattggtgctgatgagaccgtccaaggacaaggaagtcggaggctgatcagcttctctctctcagatttccaagccatggggttgaagaaagggatgtttttcaacccagacccttatctgaagatttccattcagcctgggaaacacagcatcttccccgccctccctcaccatggacaggagaggagatccaagatcataggcaacaccgtgaaccccatctggcaggccgagcaattcagttttgtgtccttgcccactgacgtgctggaaattgaggtgaaggacaagtttgccaagagccgccccatcatcaagcgcttcttgggaaagctgtcgatgcccgttcaaagactcctggagagacacgccataggggatagggtggtcagctacacacttggccgcaggcttccaacagatcatgtgagtggacagctgcaattccgatttgagatcacttcctccatccacccagatgatgaggagatttccctgagtaccgagcctgagtcagcccaaattcaggacagccccatgaacaacctgatggaaagcggcagtggggaacctcggtctgaggcaccagagtcctctgagagctggaagccagagcagctgggtgagggcagtgtccccgatggtccagggaaccaaagcatagagctttccagaccagctgaggaagcagcagtcatcacggaggcaggagaccagggcatggtctctgtgggacctgaaggggctggggagctcctggcccaggtgcaaaaggacatccagcctgcccccagtgcagaagagctggccgagcagctggacctgggtgaggaggcatcagcactgctgctggaagacggtgaagccccagccagcaccaaggaggagcccttggaggaggaagcaacgacccagagccgggctggaagggaagaagaggagaaggagcaggaggaggagggagatgtgtctaccctggagcagggagagggcaggctgcagctgcgggcctcggtgaagagaaaaagcaggccctgctccttgcctgtgtccgagctggagacggtgatcgcgtcagcctgcggggaccccgagaccccgcggacacactacatccgcatccacaccctgctgcacagcatgccctccgcccagggcggcagcgcggcagaggaggaggacggcgcggaggaggagtccaccctcaaggactcctcggagaaggatgggctcagcgaggtggacacggtggccgctgacccgtctgccctggaagaggacagagaagagcccgagggggctactccaggcacggcgcaccctggccactccgggggccacttccccagcctggccaatggcgcggcccaggatggcgacacgcaccccagcaccgggagcgagagcgactccagccccaggcaaggcggggaccacagttgcgagggctgtgacgcgtcctgctgcagcccctcgtgctacagctcctcgtgctacagcacgtcctgctacagcagctcgtgctacagcgcctcgtgctacagcccctcctgctacaacggcaacaggttcgccagccacacgcgcttctcctccgtggacagcgccaagatctccgagagcacggtcttctcctcgcaagacgacgaggaggaggagaacagcgcgttcgagtcggtacccgactccatgcagagccctgagctggacccggagtccacgaacggcgctgggccgtggcaagacgagctggccgcccctagcgggcacgtggaaagaagcccggaaggtctggaatcccccgtggcaggtccaagcaatcggagagaagactgggaagctcgaattgacagccacgggcgggtcttttatgtggaccacgtgaaccgcacaaccacctggcagcgtccgacggcagcagccaccccggatggcatgcggagatcggggtccatccagcagatggagcaactcaacaggcggtatcaaaacattcagcgaaccattgcaacagagaggtccgaagaagattctggcagccaaagctgcgagcaagccccagcaggaggaggcggaggtggagggagtgactcagaagccgaatcttcccagtccagcttagatctaaggagagaggggtcactttctccagtgaactcacaaaaaatcaccttgctgctgcagtccccagcggtcaagttcatcaccaaccccgagttcttcactgtgctacacgccaattatagtgcctaccgagtcttcaccagtagcacctgcttaaagcacatgattctgaaagtccgacgggatgctcgcaattttgaacgctaccagcacaaccgggacttggtgaatttcatcaacatgttcgcagacactcggctggaactgccccggggctgggagatcaaaacggaccagcagggaaagtcttttttcgtggaccacaacagtcgagctaccactttcattgacccccgaatccctcttcagaacggtcgtcttcccaatcatctaactcaccgacagcacctccagaggctccgaagttacagcgccggagaggcctcagaagtttctagaaacagaggagcctctttactggccaggccaggacacagcttagtagctgctattcgaagccaacatcaacatgagtcattgccactggcatataatgacaagattgtggcatttcttcgccagccaaacatttttgaaatgctgcaagagcgtcagccaagcttagcaagaaaccacacactcagggagaaaatccattacattcggactgagggtaatcacgggcttgagaagttgtcctgtgatgcggatctggtcattttgctgagtctctttgaagaagagattatgtcctacgtccccctgcaggctgccttccaccctgggtatagcttctctccccgatgttcaccctgttcttcacctcagaactccccaggtttacagagagccagtgcaagagccccttccccctaccgaagagactttgaggccaagctccgcaatttctacagaaaactggaagccaaaggatttggtcagggtccggggaaaattaagctcattattcgccgggatcatttgttggagggaaccttcaatcaggtgatggcctattcgcggaaagagctccagcgaaacaagctctacgtcacctttgttggagaggagggcctggactacagtggcccctcgcgggagttcttcttccttctgtctcaggagctcttcaacccttactatggactctttgagtactcggcaaatgatacttacacggtgcagatcagccccatgtccgcatttgtagaaaaccatcttgagtggttcaggtttagcggtcgcatcctgggtctggctctgatccatcagtaccttcttgacgctttcttcacgaggcccttctacaaggcactcctgagactgccctgtgatttgagtgacctggaatatttggatgaggaattccaccagagtttgcagtggatgaaggacaacaacatcacagacatcttagacctcactttcactgttaatgaagaggtttttggacaggtcacggaaagggagttgaagtctggaggagccaacacacaggtgacggagaaaaacaagaaggagtacatcgagcgcatggtgaagtggcgggtggagcgcggcgtggtacagcagaccgaggcgctggtgcgcggcttctacgaggttgtagactcgaggctggtgtccgtgtttgatgccagggagctggagctggtgatagctggcaccgcggaaatcgacctaaatgactggcggaataacactgagtaccggggaggttaccacgatgggcatcttgtgatccgctggttctgggctgcggtggagcgcttcaataatgagcagaggctgagattactgcagtttgtcacgggaacatccagcgtgccctacgaaggcttcgcagccctccgtgggagcaatgggcttcggcgcttctgcatagagaaatgggggaaaattacttctctccccagggcacacacatgcttcaaccgactggatcttccaccgtatccctcgtactccatgttgtatgaaaagctgttaacagcagtagaggaaaccagcacctttggacttgagtgaggacatggaacctcgcctgacattttcctggccagtgacatcacccttcctgggatgatccccttttccctttcccttaatcaactctcctttgattttggtattccatgatttttattttcaaac

SEQ ID NO: 56 - DNA, 2715 bp, Homo sapiens
  source 1..2715: mol_type = genomic DNA
agagttccatcagagcctgcagtggatgaaagacaatgatatccatgacatcctagacctcacgttcactgtgaacgaagaagtatttgggcagataactgaacgagaattaaagccagggggtgccaatatcccagttacagagaagaacaagaaggagtacatcgagaggatggtgaagtggaggattgagaggggtgttgtacagcaaacagagagcttagtgcgtggcttctatgaggtggtggatgccaggctggtatctgtttttgatgcaagagaactggaattggtcatcgcaggcacagctgaaatagacctaagtgattggagaaacaacacagaatatagaggaggataccatgacaatcatattgtaattcggtggttctgggctgcagtggaaagattcaacaatgaacaacgactaaggttgttacagtttgttacaggcacatccagcattccctatgaaggatttgcttcactccgagggagtaacggcccaagaagattctgtgtggagaaatgggggaaaatcactgctcttcccagagcgcatacatgttttaaccgtctggatctgcctccctacccatccttttccatgctttatgaaaaactgttgacagcagttgaagaaaccagtacttttggacttgagtgacctggaagctgaatgcccatctctgtggacaggcagtttcagaagctgccttctagaagaatgattgaacattggaagtttcaagaggatgcttcctttaggataaagctacgtgctgttgttttccaggaacaagtgctctgtcacatttggggactggagatgagtcctcttggaaggatttgggtgagcttgatgcccagggaacaacccaaccgtctttcaatcaacagttcttgactgccaaactttttccatttgttatgttccaagacaaagatgaacccatacatgatcagctccacggtaatttttagggactcaggagaatcttgaaacttacccttgaacgtggttcaagccaaactggcagcatttggcccaatctccaaattagagcaagttaaataatataataaaagtaaatatatttcctgaaagtacattcatttaagccctaagttataacagaatattcatttcttgcttatgagtgcctgcatggtgtgcaccataggtttccgctttcatgggacatgagtgaaaatgaaaccaagtcaatatgaggtacctttacagatttgcaataagatggtctgtgacaatgtatatgcaagtggtatgtgtgtaattatggctaaagacaaaccattattcagtgaattactaatgacagattttatgctttataatgcatgaaaacaattttaaaataactagcaattaatcacagcatatcaggaaaaagtacacagtgagttctgtttattttttgtaggctcattatgtttatgttctttaagatgtatataagaacctacttatcatgctgtatgtatcactcattccattttcatgttccatgcatactcgggcatcatgctaatatgtatccttttaagcactctcaaggaaacaaaagggccttttatttttataaaggtaaaaaaaattccccaaatattttgcactgaatgtaccaaaggtgaagggacattacaatatgactaacagcaactccatcacttgagaagtataatagaaaatagcttctaaatcaaacttccttcacagtgccgtgtctaccactacaaggactgtgcatctaagtaataattttttaagattcactatatgtgatagtatgatatgcatttatttaaaatgcattagactctcttccatccatcaaatactttacaggatggcatttaatacagatatttcgtatttcccccactgctttttatttgtacagcatcattaaacactaagctcagttaaggagccatcagcaacactgaagagatcagtagtaagaattccattttccctcatcagtgaagacaccacaaattgaaactcagaactatatttctaagcctgcattttcactgatgcataattttcttattaatattaagagacagtttttctatggcatctccaaaactgcatgacatcactagtcttacttctgcttaattttatgagaaggtattcttcattttaattgcttttgggattactccacatctttgtttatttcttgactaatcagattttcaatagagtgaagttaaattgggggtcataaaagcattggattgacatatggtttgccagcctatgggtttacaggcattgcccaaacatttctttgagatctatatttataagcagccatggaattcctattatgggatgttggcaatcttacattttatagaggtcatatgcatagttttcataggtgttttgtaagaactgattgctctcctgtgagttaagctatgtttactactgggaccctcaagaggaataccacttatgttacactcctgcactaaaggcacgtactgcagtgtgaagaaatgttctgaaaaagggttatagaaatctggaaataagaaaggaagagctctctgtattctataattggaagagaaaaaaagaaaaacttttaactggaaatgttagtttgtacttattgatcatgaatacaagtatatatttaattttgcaaaaaaaaaaaaaaaaaaaaaaag

SEQ ID NO: 57 - RNA, 57 nt, unidentified
  source 1..57: mol_type = unassigned RNA
ggtctctctggttagaccagatctgagcctgggagctctctggctaactagggaacc

SEQ ID NO: 58 - DNA, 85 bp, synthetic construct
  source 1..85: mol_type = other DNA
ccggggctcgttgagctcattagctccgagcccggtgtcatctatgtgatcttctcgagaagatcacatagatgacaccgttttt

SEQ ID NO: 59 - DNA, 3924 bp, synthetic construct
  source 1..3924: mol_type = other DNA
caactttgtatagaaaagttggagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggctcgttgagctcattagctccgagcccggtgtcatctatgtgatcttctcgagaagatcacatagatgacaccgtttttcaagtttgtacaaaaaagcaggctgggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggacccagctttcttgtacaaagtgggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgccgcggccgc

SEQ ID NO: 60 - DNA, 57 bp, synthetic construct
  source 1..57: mol_type = other DNA
agcggcccgcgcccgcgcggcacccgcggcaaaggccgccgcattcgccgccgcggc

SEQ ID NO: 61 - DNA, 15 bp, synthetic construct
  source 1..15: mol_type = other DNA
ggcggcagcagcggc

SEQ ID NO: 62 - DNA, 5996 bp, synthetic construct
  source 1..5996: mol_type = other DNA
caactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggggcgagtgcagctcttcgagatcagcctgagccacggccgcgtcgtctacagccccggggagccgttggctgggaccgtgcgcgtgcgcctgggggcaccgctgccgttccgagccatccgggtgacctgcataggttcctgcggggtctccaacaaggctaatgacacagcgtgggtagtggaggagggttacttcaacagttccctgtcgctggcagacaaggggagcctgcccgctggagagcacagcttccccttccagttcctgcttcctgccactgcacccacgtcctttgagggtcctttcgggaagatcgtgcaccaggtgagggccgccatccacacgccacggttttccaaggatcacaagtgcagcctcgtgttctatatcttgagccccttgaacctgaacagcatcccagacattgagcaacccaacgtggcctctgccaccaagaagttctcctacaagctggtgaagacgggcagcgtggtcctcacagccagcactgatctccgcggctatgtggtggggcaggcactgcagctgcatgccgacgttgagaaccagtcaggcaaggacaccagccctgtggtggccagtctgctgcagaaagtgtcctataaggccaagcgctggatccacgacgtacggaccattgcggaggtggagggtgcgggcgtcaaggcctggcggcgggcgcagtggcacgagcagatcctggtgcctgccttgccccagtcggccctgccgggctgcagcctcatccacatcgactactacttacaggtctctctgaaggcgccggaagctactgtgaccctcccggtcttcattggcaatattgctgtgaaccatgccccagtgagcccccggccaggcctggggctgcctcctggggccccacccctggtggtgccttccgcaccaccccaggaggaggctgaggctgaggctgcggctggcggcccccacttcttggaccccgtcttcctctccaccaagagccattcgcagcggcagcccctgctggccaccttgagttctgtgcctggtgcgccggagccctgccctcaggatggcagccctgcctcacacccgctgcaccctcccttgtgcatttcaacaggtgccactgtcccctactttgcagagggctccggggggccagtgcccactaccagcaccttgattcttcctccagagtacagttcttggggctacccctatgaggccccaccgtcttatgagcagagctgcggcggcgtggaacccagcctgacccctgagagcggcggcagcagcggcagcggcccgcgcccgcgcggcacccgcggcaaaggccgccgcattcgccgccgcggctaaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgccgcggccgc

SEQ ID NO: 63 - DNA, 86 bp, Homo sapiens
  source 1..86: mol_type = genomic DNA
atgacatatcccagcattggaccagcccctgaataaactggaaagacgcctggtctggcttcagttacagggagcaccaccaggga